(19) United States
(12) Patent Application Publication
(10) Pub. No.: US 2003/0077664 A1
(43) Pub. Date: Apr. 24, 2003

(54) Methods of screening for compounds that modulate hormone receptor activity

(76) Inventors: Yi Zhao, Irvine, CA (US); Scott M. Thacher, Costa Mesa, CA (US); Jia-Hao Xiao, Irvine, CA (US); Jyotirmoy Kusari, Irvine, CA (US); Roshantha A. Chandraratna, Laguna Hills, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/922,226

(22) Filed: Aug. 2, 2001

Related U.S. Application Data
(60) Provisional application No. 60/284,797, filed on Apr. 18, 2001.

(51) Int. Cl. G01N 33/53; G01N 33/567

(52) U.S. Cl. 435/7.2

(57) ABSTRACT

The present invention provides methods for identifying an effective agent that modulates a biological activity of a nuclear hormone receptor. In a method of the invention, an isolated receptor-containing complex is assayed for an altered modification state as compared to a control modification state. The presence of an altered modification state serves to identify an effective agent that modulates a biological activity of the nuclear hormone receptor.

This application is based on, and claims the benefit of, U.S. Provisional Application No. 60/284,797, filed Apr. 18, 2001, and entitled NOVEL METHODS OF SCREENING FOR COMPOUNDS THAT MODULATE HORMONE RECEPTOR ACTIVITY, and which is incorporated herein by reference.

### BACKGROUND OF THE INVENTION

1. Field of the Invention

The present invention relates generally to the fields of biochemistry and molecular medicine and, in particular, to drugs that regulate post-translational modifications of nuclear hormone receptors or associated proteins.

2. Background Information

Nuclear hormone receptors are a large family of gene regulatory, DNA-binding proteins that bind hormonally and nutritionally derived lipophilic ligands. Over 300 nuclear hormone receptors have been identified to date, including, for example, the retinoid X receptor, retinoic acid receptor, progesterone receptor, estrogen receptor, androgen receptor and vitamin D receptor (Whitfield et al., J. Cell. Biochem. Suppl. 32/33:110-122 (1999); Laudet et al., Cell 97:161-163 (1999); and Sluder et al., Genome Res. 9:103-120 (1999)). Nuclear hormone receptors have been conserved throughout evolution and play a role in cell growth and proliferation, development and homeostasis. Not surprisingly, nuclear hormone receptors have been implicated in disease. Retinoic acid receptors can play a role in, for example, acute promyelocytic leukemia and acne; thyroid hormone receptor is involved in thyroid hormone resistance and hypercholesterolemia; vitamin D receptors play a role in type 2D-dependent rickets and osteoporosis; peroxisome proliferator activated receptor (PPAR) contributes to obesity and Type II diabetes; and the estrogen receptor plays a role in some forms of breast cancer (Lazar, J. Invest. Medicine 47:364-368 (1999)). Progress has been made in understanding the role of nuclear hormone receptors and their ligands in disease, and in identifying hormone receptor ligands with therapeutic activity.

In the case of the retinoid receptors, retinoid ligands have been developed as therapeutics for a variety of disorders. Current retinoid therapies include differentiation of acute promyelocytic leukemia (APL); treatment of nodulocystic acne, a severe form of inflammatory acne; treatment of psoriasis; prevention of secondary head and neck cancers; topical therapy of acne vulgaris; and reversal of UV-mediated photodamage (Thacher et al., Current Pharm. Design 6:25-58 (2000)). Unfortunately, the dosage of these retinoid ligands is limited by significant side effects, including irritation and inflammation of skin and mucous membranes, elevation of serum triglycerides, dysregulation of bone formation and resorption, headaches, hypothyroidism, and fetal malformation. Thus, there is a need for a new generation of retinoid and other hormone-based therapeutics which can have, for example, greater selectivity and fewer side effects.

Nuclear hormone receptors have long been known to be DNA-binding proteins that can activate or repress transcription of target genes. In most cases, transcriptional activity of the hormone receptor is controlled in a ligand-dependent manner. Current assays for identifying therapeutic ligands are based on the transcriptional activity of the nuclear hormone receptor of interest. However, compounds identified using these assays often are characterized by significant side effects.

Thus, there is a need for novel assays which can be used to identify therapeutic hormone receptor ligands but which do not rely on the transcriptional activity of the nuclear hormone receptor. The present invention satisfies this need and provides related advantages as well.

### SUMMARY OF THE INVENTION

The present invention provides a method for identifying an effective agent that modulates a biological activity of a nuclear hormone receptor. The method includes the steps of contacting the nuclear hormone receptor with one or more agents and a eukaryotic cell sample to form a test sample under conditions suitable to form a receptor-containing complex; isolating the receptor-containing complex from the test sample; providing to the isolated receptor-containing complex conditions suitable for modification of the receptor-containing complex; and assaying the isolated receptor-containing complex for an altered modification state occurring in the isolated receptor-containing complex as compared to a control modification state, where the presence of the altered modification state indicates that at least one of the one or more agents is an effective agent that modulates a biological activity of the nuclear hormone receptor. In a method of the invention, the altered modification state can be, for example, an increased or decreased phosphorylation state. In particular embodiments, a method of the invention is practiced by assaying for an altered phosphorylation state of a nuclear hormone receptor or an altered phosphorylation state of a 160 kDa protein.

In a method of the invention, a nuclear hormone receptor is contacted with one or more agents and a eukaryotic cell sample. In one embodiment, the one or more agents with which the nuclear hormone receptor is contacted are present during isolation of the receptor-containing complex from the test sample.

In a method of the invention, the isolated receptor-containing complex is provided with conditions suitable for modification of the receptor-containing complex, for example, for phosphorylation of the receptor-containing complex. In one embodiment, the conditions are a magnesium concentration of 1 to 25 mM.

The methods of the invention rely on a nuclear hormone receptor. In particular embodiments, the nuclear hormone receptor is a retinoid X receptor (RXR), hepatocyte nuclear factor 4 (HNF4), testicular receptor, tailless gene homolog (TLX), chicken ovalbumin upstream promoter transcription factor (COUP-TF), thyroid receptor (TR), retinoic acid receptor (RAR), peroxisome proliferator activated receptor (PPAR), reverse Erb (revErb), RAR-related orphan receptor (ROR), steroidogenic factor-1 (SF-1), liver receptor homolog-1 (LRH-1), liver X receptor (LXR), farnesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor related 1 (NURR1), estrogen receptor (ER), estrogen-related receptor (ERR), glucocorticoid receptor (GR), androgen receptor (AR), progesterone receptor (PR) or mineralocorticoid receptor (MR). In one embodiment, the nuclear hormone receptor is a retinoid X receptor, retinoic acid receptor, progesterone receptor, estrogen receptor, androgen receptor or vitamin D receptor. In another embodiment, the nuclear hormone receptor is a retinoid X receptor such as RXRα, RXRβ or RXRγ.

A nuclear hormone receptor useful in a screening method of the invention also can be a truncated nuclear hormone receptor. Such a truncated receptor can be, for example, a truncated hormone receptor lacking a functional DNA-binding domain or a truncated nuclear hormone receptor containing at least the ligand-binding domain of the receptor. In other embodiments, a nuclear hormone receptor useful in the invention is a fusion protein that contains a heterologous sequence from a different nuclear hormone receptor or from a protein that is not a nuclear hormone receptor. A nuclear hormone receptor useful in the invention can be a fusion protein that contains, for example, a heterologous membrane-anchoring domain, heterologous epitope tag or heterologous protein kinase recognition sequence, or any combination of these heterologous sequences. A nuclear hormone receptor useful in the invention also can be a variant with an increased ratio of cytoplasmic to nuclear localization as compared to wild type nuclear hormone receptor, or a variant that lacks a functional DNA-binding domain.

In a method of the invention, isolation of the receptor-containing complex can be achieved using a variety of means including specific binding to the receptor-containing complex, for example, immunoprecipitation of the receptor-containing complex. Immunoprecipitation can be performed, for example, using antibody immunoreactive with the nuclear hormone receptor.

A method of the invention can be practiced with a variety of eukaryotic cell samples, including viable cells, which can be, for example, transiently or stably transfected; a whole cell lysate; or a fractionated cell lysate. The test sample containing the nuclear hormone receptor and one or more agents to be assayed also can include, if desired, an exogenous heterodimeric partner of the nuclear hormone receptor, or an exogenous kinase that enhances detection of an altered modification state. In one embodiment, the isolated receptor-containing complex includes a serine/threonine kinase.

The present invention also provides a method for identifying an effective agent that modulates a biological activity of a retinoid X receptor. The method includes the steps of contacting the retinoid X receptor and a eukaryotic cell sample with one or more agents to form a test sample; and assaying a protein in the test sample for an altered modification state as compared to a control modification state, where the presence of the altered modification state indicates that at least one of the one or more agents is an effective agent that modulates a biological activity of the retinoid X receptor.

Further provided by the invention is a method for identifying an improved effective agent that modulates a biological activity of a nuclear hormone receptor. The method includes the steps of contacting the nuclear hormone receptor with one or more agents and a eukaryotic cell sample to form a test sample under conditions suitable to form a receptor-containing complex; isolating the receptor-containing complex from the test sample; providing to the isolated receptor-containing complex conditions suitable for modification of the receptor-containing complex; assaying the isolated receptor-containing complex, or a component thereof, for an altered modification state occurring in the isolated receptor-containing complex as compared to a control modification state; and assaying for direct transcriptional activity of the nuclear hormone receptor contacted with the one or more agents, where the presence of an altered modification state combined with the absence of direct transcriptional activity indicates that at least one of the one or more agents is an improved effective agent that modulates a biological activity of the nuclear hormone receptor.

The present invention also provides a method for identifying an effective agent that modulates protein kinase A activity associated with a nuclear hormone receptor. A method of the invention is practiced by contacting nuclear hormone receptor with one or more agents and a cell sample to form a test sample under conditions suitable to form a receptor-containing complex; isolating the receptor-containing complex from the test sample; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. In a method of the invention for identifying an effective agent that modulates protein kinase A activity associated with a nuclear hormone receptor, the altered phosphorylation state can be an increased or decreased phosphorylation state.

A substrate useful in a method of the invention can be, for example, a purified substrate and, in one embodiment, has a Km of less than 20 μM for protein kinase A. Peptide substrates, including purified peptide substrates, are useful in the methods of the invention and, in one embodiment, the peptide substrates have at most ten residues. A peptide substrate useful in the invention can include, for example, the amino acid sequence Arg-X-Ser, Arg-Arg-X-Ser, Arg-X-X-Ser, Lys-Arg-X-X-Ser or Arg-X-Lys-Arg-X-X-Ser-X (SEQ ID NO: 113), where X is independently any amino acid. A peptide substrate useful in the methods of the invention can include, for example, the sequence Arg-Arg-X-Ser and, in particular embodiments, can contain the sequence LRRASLG (SEQ ID NO: 59) or GRTGRRNSI (SEQ ID NO: 60). In particular embodiments, such a peptide substrate has a length of at most ten residues. A method of the invention also can be practiced with purified protein substrates, for example, myelin basic protein.

The invention also provides a method for identifying an effective agent that modulates protein kinase A activity associated with a nuclear hormone receptor by contacting the nuclear hormone receptor with one or more agents and a cell sample to form a test sample under conditions suitable to form a receptor-containing complex, where the receptor is retinoid X receptor (RXR), retinoic acid receptor (RAR) or peroxisome proliferator activated receptor (PPAR); isolating the receptor-containing complex from the test sample; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. In one embodiment, the nuclear hormone receptor is a retinoid X receptor (RXR) and can be, for example RXRα.

A method of the invention also can be practiced with a nuclear hormone receptor, which is a variant or fusion protein rather than wild type receptor. In one embodiment, the nuclear hormone receptor is a variant with an increased ratio of cytoplasmic to nuclear localization as compared to wild type nuclear hormone receptor. In another embodiment, the nuclear hormone receptor is a fusion protein that contains a heterologous membrane-anchoring domain. In a further embodiment, the nuclear hormone receptor is a fusion protein containing a pleckstrin homology domain. In yet another embodiment, the nuclear hormone receptor is a fusion protein containing a heterologous epitope tag.

A method of the invention for identifying an effective agent that modulates protein kinase A activity associated with a nuclear hormone receptor can be practiced with endogenous or exogenous receptor. In one embodiment, the invention is practiced with a cell sample containing an exogenous nucleic acid molecule encoding the nuclear hormone receptor. In another embodiment, the nuclear hormone receptor is endogenous to the cell sample. A cell sample to be used in a screening methods of the invention can contain an exogenous nucleic acid molecule encoding a heterodimeric partner of the nuclear hormone receptor or an exogenous nucleic acid molecule encoding a catalytic subunit of protein kinase A. In such a cell sample, the nuclear hormone receptor can be endogenous or exogenous to the cell.

A method of the invention can be practiced with a variety of types of cell samples. In one embodiment, the cell sample contains viable eukaryotic cells, and, in another embodiment, the cell sample is a eukaryotic whole cell sample. In one embodiment, isolation of the substrate is performed in the presence of the one or more agents. In a method of the invention, isolation of the receptor-containing complex can be performed by a variety of means, for example, by specific binding to the receptor-containing complex.

Further provided by the invention is a method for identifying an effective agent that modulates protein kinase A activity associated with a nuclear hormone receptor by contacting the nuclear hormone receptor with one or more agents and a cell sample to form a test sample under conditions suitable to form a receptor-containing complex; immunoprecipitating the receptor-containing complex from the test sample to isolate the receptor-containing complex; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. Immunoprecipitation can be performed, for example, using antibody immunoreactive with the nuclear hormone receptor. Where the nuclear hormone receptor is a fusion protein containing a heterologous epitope tag, the immunoprecipitation can be performed, for example, using antibody immunoreactive with the epitope tag. A variety of means can be used to assay the protein kinase A substrate for an altered phosphorylation state including, for example, detecting radiolabeled substrate.

### BRIEF DESCRIPTION OF THE DRAWINGS

FIG. 1 shows the amino acid sequences of human RXRα (SEQ ID NO: 1), human RXRβ (SEQ ID NO: 2) and human RXRγ (SEQ ID NO: 3).

FIG. 2 shows a schematic view of nuclear receptor functional domains. Modular diagrams in the top panel are drawn to scale and aligned at the conserved E1 domain. The DNA-binding region consists of two (Cys)₄-type zinc-finger motifs (C4 Zn fingers), followed by a C-terminal extension (CTE) of varying length. Dimerization and ligand-binding contacts determined by X-ray crystallography also are shown in the top panel. The center panel shows a selected portion of the DNA-binding domain for several receptors, with solid circles indicating DNA contacts as determined by X-ray crystallography for human RXRα, human TRβ, human ERα and rat GR. Jellyfish RXR is shown for comparison. The lower panel details three subregions of the ligand-binding domain in several nuclear hormone receptors, including the conserved E1 domain that supports dimerization and participates in transactivation; h9, which participates in dimerization; and the AF2 region, which contains ligand contacts and effects transactivation. Residues highly conserved among all nuclear hormone receptors are present in the E1 subregion and highlighted. SEQ ID NOS: are shown in parenthesis.

FIG. 3 shows activation of MAP kinases by RXR-specific agonist AGN194204. (A) Activation of MAP kinases (MAPKs) by AGN194204 in 3T3-L1 fibroblasts. 3T3-L1 fibroblasts were starved in DMEM with 0.1% calf bovine serum for overnight and then stimulated with vehicle (DMSO, lane 0), 30 nM insulin (lane 1), 100 nM AGN194204 (lane 2), 30 nM insulin plus 100 nM AGN194204 (lane 3) for 5 minutes, or 100 nM AGN194204 for 30 minutes followed by the additional of 30 nM insulin for 5 minutes (lane 4). Cells were lysed directly with 1×SDS sample buffer and the cell lysates were separated by SDS-PAGE and immunoblotted with anti-phospho-MAPK. (B) Activation of MAP kinase by AGN194204 in 3T3 differentiated adipocytes. Differentiated 3T3 adipocytes were starved, stimulated, and manipulated as described above. (C) Dose and time dependent activation of MAP kinases by AGN194204 in 3T3-L1 fibroblasts. Cells were starved as described above, then stimulated by different doses of AGN194204 (as indicated in left panel) for 5 minutes and for variable time (as indicated in right panel) at 10⁻¹⁰ M concentration. Detection of MAP kinase phosphorylation was performed as described above with anti-phospho-MAPK from Promega.

FIG. 4 shows the effect of MAPK pathway on epitope-tagged RXR in transfected HEK 293 cells. The top panel shows Western blotting with anti-RXR antibody (D20). The middle panel shows Western blotting with anti-Flag antibody (M2). The bottom panel shows Western blotting with anti-phospho-MAPK antibody.

FIG. 5 shows that RXRα associates with a protein kinase in the presence of RXR-specific ligand. (A) AGN194204-dependent protein kinase activity in Flag-RXR immunocomplexes, which were immunoprecipitated from transfected cells using antibody against the “Flag” epitope tag fused to the RXR receptor. HEK 293 cells were transiently transfected with Flag-RXR expression vector and stimulated without (−) or with (+) AGN194204 (10⁻⁷ M) for 10 minutes. Total cell lysates were immunoprecipitated with anti-Flag antibody (M2). Aliquots of the immunocomplexes were used for the in vitro kinase reaction with [γ-³²P]ATP and separated on SDS-PAGE. Phosphorylated proteins were detected by autoradiography (top panel). Additional aliquots of the immunocomplexes were subjected to immunoblotting with polyclonal anti-RXR antisera (D20; bottom panel). (B) Phosphoamino acid analysis. Flag-RXR and the 160-kDa protein were phosphorylated in vitro and separated on SDS-PAGE as described above, then transferred onto PVDF membrane, detected by autoradiography, and cut out for hydrolysis with acid (6 N HCl). The phosphorylated amino acids and standards (P-Ser, P-Thr, and P-Tyr) were separated by thin-layer chromatography. Positions of the standards P-Ser, P-Thr, and P-Tyr were visualized by ninhydrin staining and are indicated by arrows. (C) In vivo metabolic labeling of transfected HEK 293 cells. HEK 293 cells were transfected with Flag-RXR expression vector. Cells were washed with phosphate-free medium and starved for 90 minutes before incubation with [³²P] orthophosphate for another 4 hours. Before harvesting, cells were stimulated with vehicle, RXR-specific ligand AGN192404, RXR-specific antagonist AGN195393, or RAR-specific agonist TTNPB for 10 minutes. Total cell lysates were immunoprecipitated with anti-Flag antibody (M2) and subjected to separation on SDS-PAGE. Phosphorylated proteins were detected by autoradiography.

FIG. 6 shows the dose and RXR ligand-dependence of in vitro kinase activity in Flag-RXR immunocomplexes. (A) Dose of AGN194204 dependent kinase activity was determined in HEK 293 cells transfected with Flag-RXR expression vector and stimulated with the indicated dose of RXR-specific ligand AGN194204 for 10 minutes. Cell lysates were immunoprecipitated with anti-Flag antibody (M2) in the absence (−) or presence (+) of AGN194204 in the immunoprecipitation buffer. In vitro kinase reaction with [γ-³²P]ATP, separation on SDS-PAGE, and detection of the phosphorylated proteins were performed as described above. (B) RXR-specific ligand dependent kinase activity. HEK 293 cells were transfected with vector alone or Flag-RXR expression vector, and stimulated with vehicle, RXR-specific ligand (AGN194204, AGN195029, AGN192620, AGN195203, and AGN195184), or RXR-specific antagonist AGN195393 for 15 minutes. Total cell lysates were subjected to immunoprecipitation, and the in vitro kinase reaction performed as described above.

FIG. 7 shows a schematic representation of the structure of RXRα deletion mutants.

FIG. 8 shows identification of RXRα regions required for association with a protein kinase. HEK 293 cells transiently transfected with Flag-RXRα or mutant expression vectors were treated with RXR-specific ligand AGN194204 for 10 minutes. Cell lysis, immunoprecipitation with anti-Flag (M2), in vitro kinase assay, and detection of the phosphorylated proteins in the RXRα immunoprecipitated complexes were performed as described above.

FIG. 9 shows identification of proteins interacting with Flag-RXR in transfected HEK 293 cells by two-dimensional gel electrophoresis. HEK 293 cells were transfected with Flag-RXR and lysed 48 hours after transfection. Immunocomplexes were prepared with anti-Flag antibody (M2), solubilized in IEF-sample buffer, and applied to separation on two-dimensional gel electrophoresis. Proteins were visualized by silver staining.

FIG. 10 shows the influence of heat shock protein 90 (HSP90) inhibition on RXR-associated kinase activity. HEK 293 cells were transfected with Flag-RXR expression vector and treated with 1 μM of the HSP90 inhibitor geldanamycin for 30 minutes or overnight as indicated. Cell lysates were immunoprecipitated with anti-Flag antibody (M2) prior to in vitro kinase reaction with [γ-³²P]ATP, separation on SDS-PAGE, and detection of the phosphorylated proteins as described above.

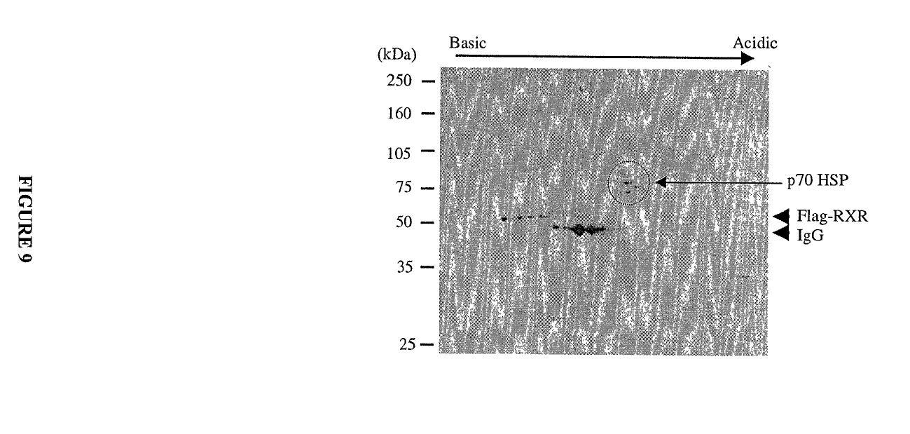

FIG. 11 shows the influence of overexpressed MEK1 mutants and protein tyrosine kinase JAK1 on RXRα-associated kinase activity in transiently transfected HEK 293 cells. (A) Influence of the MAPK pathway on the kinase in the FLAG-RXR immunocomplexes. HEK 293 cells were cotransfected with Flag-RXR expression vector, together with vector alone, wild-type MEK1, constitutively active MEK1 mutant (CA), or dominant negative MEK1 mutant (DN). Transfected cells were lysed 48 hours after transfection with or without prior stimulation with AGN194204 for 10 minutes. Preparation of immunocomplexes and in vitro kinase reactions were carried out as described above. (B) Influence of overexpressed JAK1 on kinase activity in Flag-RXR immunocomplexes. HEK 293 cells were transfected with vector alone (lane 1) or Flag-RXR (lanes 2-8) plus varying concentrations of JAK1 expression vector (lane 2: 0 ng; lane 3: 8 ng; lane 4: 24 ng; lane 5: 74 ng; lane 6: 220 ng; lane 7: 670 ng; lane 8: 2 μg). Transfected cells were lysed following incubation with AGN194204 for 10 minutes; in vitro kinase reactions were then performed as described above.

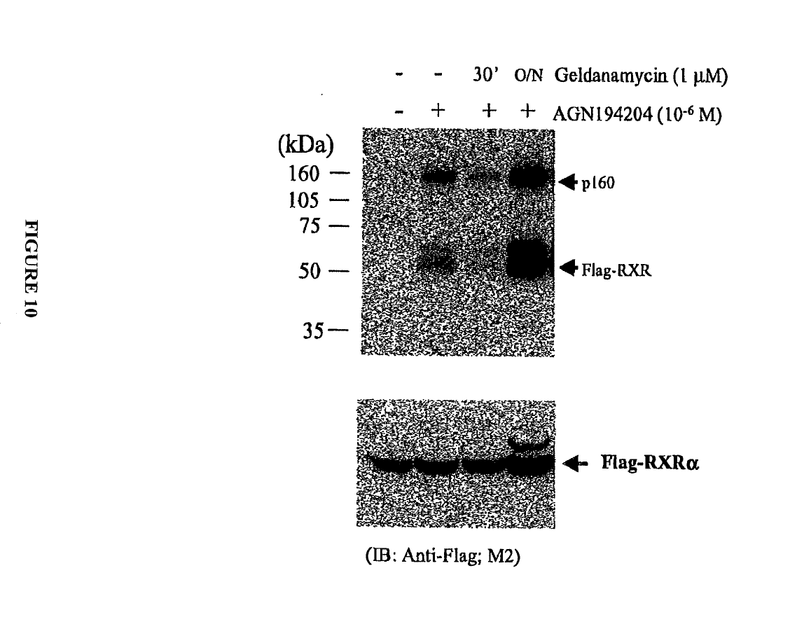

FIG. 12 shows pharmacokinetic analysis of RXR compounds in induction of kinase activity using a gel-based kinase assay. (A) Phosphorylation of 160 kDa protein. (B) Phosphorylation of RXRα. Stable RXRα-expressing 293 cells were treated with compounds at indicated doses for 10 minutes. Cells were then harvested and analyzed by gel-based kinase assays using γ-³²P-ATP as described in FIG. 5A. Phosphorylated proteins were quantified by exposing gels to a PhosphorImager (Molecular Dynamics).

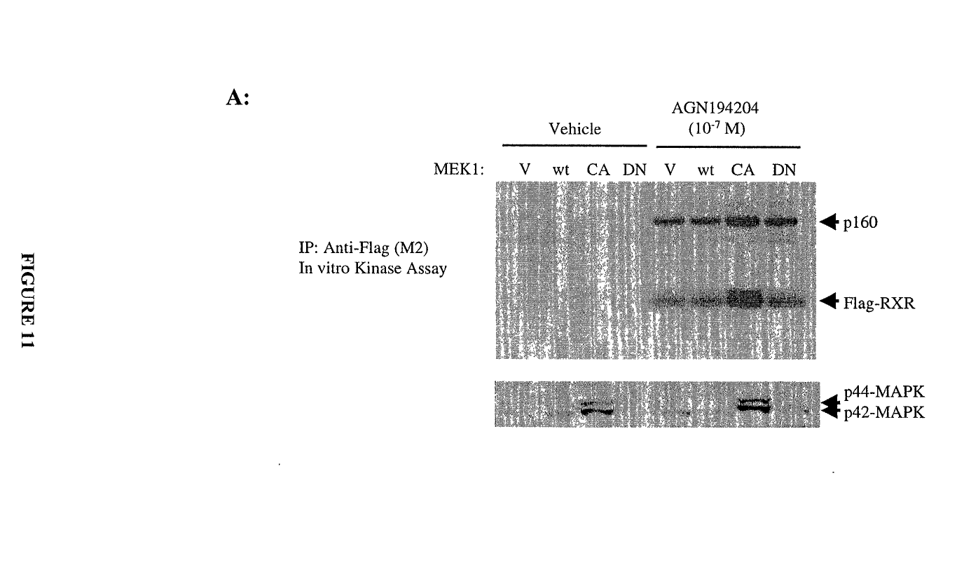

FIG. 13 shows a high throughput kinase assay for screening nuclear hormone receptor compounds capable of activating kinase activity. (A) This panel illustrates the principle of a multiple-well plate-based kinase assay using RXR receptor. Extracts are prepared from Flag-RXR-overexpressing cells and added to a multiple-well plate in which the wells are coated with scintillation materials and protein A. In the presence of RXR compounds, RXR forms complexes with kinases and p160 protein. Addition of anti-Flag antibodies enables the complexes to be brought to the proximity of the scintillation materials on the well wall and specifically attached to the plate. Non-specific proteins are washed away by buffer. The kinase reaction is initiated by adding kinase buffer containing γ-³³P-ATP. After the reaction, phosphorylated complexes remain attached to the plate while free γ-³³P-ATP is washed away. Radioactivity generated from the phosphorylated proteins is quantified using a plate-format scintillation counter, which represents the compound activity. (B) An exemplary high throughput assay. 293 cells were transfected with Flag-RXRα or Flag-PH-RXRα and treated with or without AGN194204. Cell lysis was performed as described in FIG. 5A. Recombinant Flag-PH-RXRα contained the plekstrin homology domain (PH) from IRS-1 between Flag and RXRα. Cell lysate (200 μl) was mixed with the anti-Flag antibody, added to each well of a 96-well Flashplate (NEN-Dupont) coated with protein A, and then incubated at 4° C. for 4 hours with gentle shaking. Approximately 200 μg total cell lysate and 0.5 μg antibody were added per well. After the incubation, the plate was washed four times with the lysis buffer with or without AGN194204. Kinase buffer (50 μl), which contains 40 mM Tris-HCL at pH 7.5, 10 mM MgCl₂, and 25 μCi γ-³³P-ATP (Amersham), were added to each well and incubated at room temperature for 20 minutes. After the reaction, the plate was washed 4 times with 200 μl of kinase buffer without the γ-³³P-ATP and then counted in the Microbeta counter (Wallac).

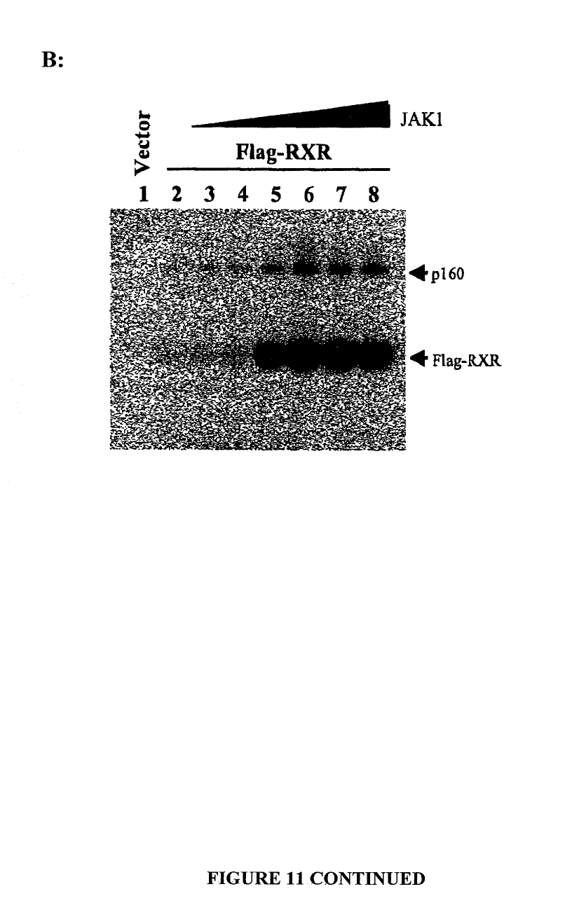

FIG. 14 shows determination of protein kinase A activity in cells transfected with RXRα or PH-RXRα. FIG. 14A shows the effect of several protein kinase inhibitors on PKA activity using kemptide (LRRASLG; SEQ ID NO: 59) as a substrate. 293 cells were transfected with PH-RXRα. Sample 1: 0.1% DMSO (10 minutes). Sample 2: 10⁻⁷ M AGN194204 (10 minutes). Sample 3: 0.1% DMSO (30 minutes). Sample 4: 10⁻⁷ M AGN194204 (30 minutes). Sample 5: 10⁻⁷ M AGN194204 (30 minutes) with inhibitors of PKC and CaMDK. Sample 6: 10⁻⁷ M AGN194204 (10 minutes) with PKA inhibitor. Sample 7: No kemptide substrate or AGN194204. Sample 8: 10⁻⁷ M AGN194204 with no kemptide substrate.

FIG. 14B shows determination of PKA activity using substrate GRTGRRNSI (SEQ ID NO: 60). Sample 1: 0.1% DMSO (10 minutes). Sample 2: 10⁻⁷ M AGN194204 (10 minutes). Sample 3: 0.1% DMSO (30 minutes). Sample 4: 10⁻⁷ M AGN194204 (30 minutes). Sample 5: No immunoprecipitate.

FIG. 14C shows determination of PKA activity in 293 cells transfected with PH-RXRα or RXRα using kemptide (LRRASLG; SEQ ID NO: 59) as a substrate. 293 cells were transfected with PH-RXRα (samples 1 to 4) or RXRα (samples 5 and 6) and treated with DMSO or AGN194204 for 10 minutes. Sample 1: 0.1% DMSO. Sample 2: 10⁻⁷ M AGN194204. Sample 3: 0.1% DMSO with PKA inhibitor. Sample 4: 10⁻⁷ M AGN194204 with PKA inhibitor. Sample 5: 0.1% DMSO. Sample 6: 10⁻⁷ M AGN194204.

FIG. 15A shows PKA activity in 3T3-L1 adipocytes in the absence and presence of AGN194204. Untransfected cells were incubated in the absence and presence of AGN194204 prior to immunoprecipitation with antibody against RXRα. Kinase activity was determined in the immunoprecipitate using kemptide (LRRASLG; SEQ ID NO: 59) as a substrate. Samples 1,3: 0.1% DMSO (10 minutes). Samples 2,4: 10⁻⁷ M AGN194204 (10 minutes).

FIG. 15B shows PKA activity in cells stably overexpressing FLAG-RXRα and transfected with the catalytic subunit of PKA. Where indicated, cells were treated with AGN194204 for 10 minutes. Sample 1: No ligand; immunoprecipitation with beads only. Sample 2: 10⁻⁷ M AGN194204; immunoprecipitation with beads only. Sample 3: No ligand; immunoprecipitation with anti-FLAG antibody. Sample 4: 10⁻⁷ M AGN194204; immunoprecipitation with anti-FLAG antibody. Sample 5: No ligand; immunoprecipitation with beads only. Sample 6: 10⁻⁷ M AGN194204; immunoprecipitation with beads only. Sample 7: No ligand; immunoprecipitation with anti-PKA antibody. Sample 8: 10⁻⁷ M AGN194204; immunoprecipitation with anti-PKA antibody.

FIG. 16 shows that the RXRα associated protein kinase phosphorylates exogenous protein substrates. HEK 293 cells were transiently transfected with vector alone or FLAG-RXR expression vector and stimulated without (−) or with (+) 10⁻⁷ M AGN194204 for 10 minutes. Total cell lysates were immunoprecipitated with anti-FLAG antibody (M2). Immunoprecipitated material was used for in vitro kinas reaction with γ-³²P-ATP alone or with addition of exogenous mixed histones or myelin basic protein (MBP). The phosphorylated proteins were separated on SDS-PAGE and detected by autoradiography.

FIG. 17 shows that RXRα is associated with and phosphorylated by protein kinase A. A. In vivo ligand independent association between RXRα and PKA. 293 cells were transfected with expression vectors encoding FLAG-RXRα and the catalytic subunit of PKA and treated with or without AGN194204 for 10 minutes. Left panel: Cells were immunoprecipitated with anti-FLAG antibody, and the immunoprecipitates analyzed by SDS-PAGE and blotting with anti-PKA antibody. Right panel: Cells were immunoprecipitated with anti-PKA antibody, and the immunoprecipitates analyzed by SDS-PAGE and blotting with anti-FLAG antibody. B. Phosphorylation of RXRα by PKA. RXRα receptors immunoprecipitated from 293 cells treated with or without AGN194204 were incubated with purified PKA and γ-ATP. PKA I and PKC I indicate inhibitors of PKA and PKC, respectively.

FIG. 18 shows exemplary pleckstrin homology (PH) domains. Amino acid identity in five or more PH domains is indicated by a black box; amino acid homology in five or more PH domains is indicated by grey shading. Plec-N, human pleckstrin, residues 1 to 105 (SEQ ID NO: 114); Plec-C, human pleckstrin, residues 239 to 350 (SEQ ID NO: 115); RasGAP, human Ras GTPase activating protein, residues 292 to 404 (SEQ ID NO: 116); Akt, human serine/threonine kinase AKT2, residues 1 to 118 (SEQ ID NO: 117); Spectrin, human β-spectrin, residues 2192 to 2311 (SEQ ID NO: 118); BARK, human β-adrenergic receptor kinase, residues 553 to 656 (SEQ ID NO: 119); Tiam1-PH1, mouse GDP-GTP exchanger Tiam-1, residues 440 to 546 (SEQ ID NO: 120); Grb7, mouse growth factor receptor bound protein 7, residues 224 to 345 (SEQ ID NO: 121); Dynamin, human GTPase dynamin 1, residues 515 to 629 (SEQ ID NO: 122); Irsl, rat insulin receptor substrate 1, residues 7 to 119 (SEQ ID NO: 123); RasGRFPH1, rat Ras guanine nucleotide release factor, residues 17 to 134 (SEQ ID NO: 124); RasGRFPH2, rat Ras guanine nucleotide release factor, residues 451 to 584 (SEQ ID NO: 125); <10> indicates omitted residues EKGKINKGRL (SEQ ID NO: 126); Dbl, human product of the dbl proto-oncogene, residues 704 to 813 (SEQ ID NO: 127); Vav, mouse product of the vav proto-oncogene, residues 397 to 508 (SEQ ID NO: 128); Sos, human son of sevenless protein, residues 438 to 548 (SEQ ID NO: 129); Bcr, human break point cluster region gene product, residues 728 to 870 (SEQ ID NO: 130); <19> indicates omitted residues GSKATERLKK KLSEQESLL (SEQ ID NO: 131); Btk, human Bruton's tyrosine kinase, residues 1 to 137 (SEQ ID NO: 132); <20> indicates omitted residues PPERQIPRRG EESSEMEQIS (SEQ ID NO: 133); PLC-delta, rat phospholipase Cδ₁, residues 15 to 134 (SEQ ID NO: 134); PLC-gamma, human phospholipase Cy₁, residues 22 to 146 (SEQ ID NO: 135); <7> indicates omitted rat residues DRYQEDP (SEQ ID NO: 136).

### DETAILED DESCRIPTION OF THE INVENTION

The present invention is directed to the surprising discovery that a kinase can be directly associated with a nuclear hormone receptor such as a retinoid X receptor, and can phosphorylate this receptor as well as associated proteins in a ligand-dependent manner. Based on these findings, the invention provides novel assays for identifying nuclear hormone receptor ligands and regulators. These assays do not depend upon transcriptional activity of the nuclear hormone receptor, nor do they require that the nuclear hormone receptor have DNA-binding activity.

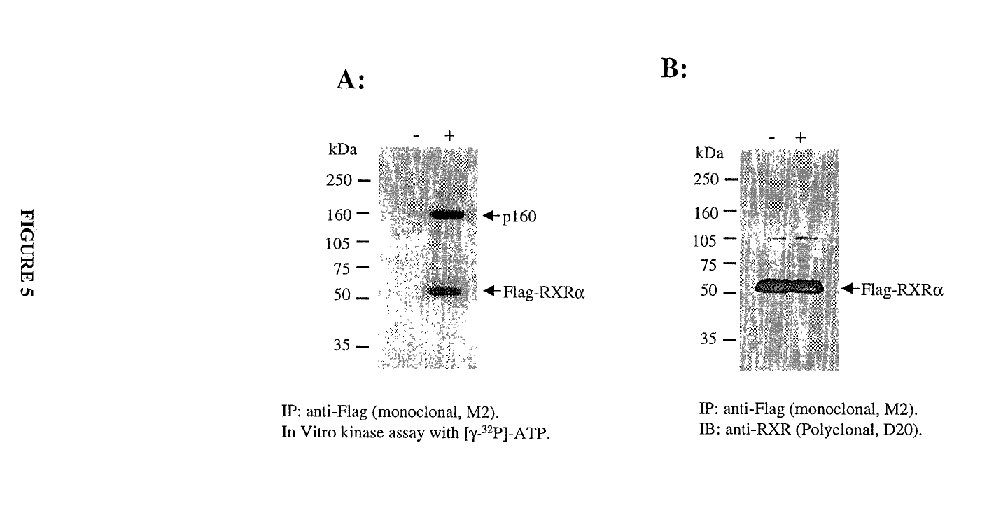

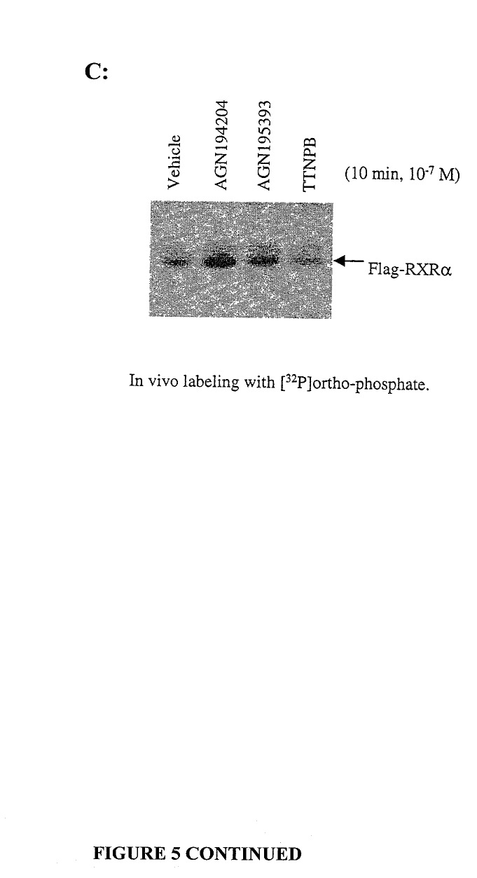

As disclosed herein, in vivo labeling with [³²P] ortho-phosphate demonstrated that retinoid X receptor a (RXRα) can be modified by phosphorylation and that this phosphorylation can be stimulated by the RXR-specific agonist AGN194204 but not by the RXR antagonist AGN195393, or the RAR-specific agonist TTNPB (see, also, Table 2). As further disclosed herein in Example IIA, HEK 293 cells were transfected with an epitope tagged Flag-RXR construct; complexes immunoprecipitated with anti-Flag antibody were resuspended in kinase buffer and used for an in vitro kinase reaction with [γ-³²P] ATP. Phosphorylation of RXR and another protein in the RXR immunocomplex of about 160 kDa was observed following treatment with RXR-specific ligand (see FIG. 5A). Phosphoamino acid analysis of [γ-³²P]ATP-labeled RXR and 160 kDa protein demonstrated that these proteins are phosphorylated on serine and threonine but not tyrosine, indicating that a serine/threonine kinase is associated with RXR (Example IIB and FIG. 5B). As shown in Example IIC, phosphorylation was dependent on any of several RXR-specific agonists but was not stimulated by the RXR-specific antagonist AGN195393 or the RAR-specific agonist AGN195183 (TTNPB), indicating that kinase activity was associated with the RXR receptor in a ligand-dependent manner (FIG. 6B). As further disclosed herein, various RXR deletion mutants containing an intact E region, which contains the critical core of the ligand-binding domain, recruited kinase activity to the immunocomplex in the presence of RXR-specific agonist, demonstrating that the RXR ligand-binding domain is sufficient for RXR interaction with the associated kinase (see Example III). Additional results disclosed herein demonstrate that cotransfection of the tyrosine kinase JAK1 with RXR in HEK 293 cells augmented ligand-dependent phosphorylation of RXRα and p160 in RXR-containing immunocomplexes.

Based on these discoveries, the present invention provides a method for identifying an effective agent that modulates a biological activity of a nuclear hormone receptor. The method includes the steps of contacting the nuclear hormone receptor with one or more agents and a eukaryotic cell sample to form a test sample under conditions suitable to form a receptor-containing complex; isolating the receptor-containing complex from the test sample; providing to the isolated receptor-containing complex conditions suitable for modification of the receptor-containing complex; and assaying the isolated receptor-containing complex for an altered modification state occurring in the receptor-containing complex as compared to a control modification state, where the presence of the altered modification state indicates that at least one of the agents is an effective agent that modulates a biological activity of the nuclear hormone receptor. In a method of the invention, the altered modification state can be, for example, an increased or decreased phosphorylation state.

In the methods of the invention, an effective agent that modulates a biological activity of a nuclear hormone receptor is identified by an altered modification state occurring in an isolated receptor-containing complex. Such an altered modification state can be, without limitation, an altered phosphorylation state of the nuclear hormone receptor such as increased phosphorylation of an RXR receptor or an altered phosphorylation state of an associated 160 kDa protein such as increased phosphorylation of a 160 kDa protein. In one embodiment, the altered modification state is any modification state other than a phosphorylation state of the cofactor TIF1α. In another embodiment, the altered modification state is any modification state other than a phosphorylation state of MAP kinase (MAPK). In a further embodiment, the altered modification is any modification state other than a phosphorylation state of thyroid hormone receptor. In yet a further embodiment, the altered modification state is any modification state other than a phosphorylation state of TIF1α, MAPK, or thyroid receptor, or any combination thereof.

As used herein, the term “altered modification state” means a post-translational modification of one or more components of the isolated receptor-containing complex which is significantly increased, decreased or qualitatively distinct from a control modification state of the same one or more components. Such an altered modification state can result from an enzymatic modification or from a nonenzymatic, chemical modification, and can be reversible or irreversible. Exemplary modification states include phosphorylation states, for example, the extent of serine, threonine, tyrosine, histidine or lysine phosphorylation; adenylation and ADP-ribosylation states; methylation states, for example, the extent of methylation at the α-amino group or on the side chains of Lys, Arg, and His; acetylation states; hydroxylation states; lipidation states; glycosylation states; and conformational states.

One skilled in the art understands that an “altered modification state” can represent a quantitative or qualitative difference as compared to a control modification state. For example, where the control modification state is a particular amount of serine phosphorylation on a component protein or proteins, an altered modification state can be, for example, an increased amount of serine phosphorylation on the same or a different residue of the same component protein or proteins, a decreased amount of serine phosphorylation on the same or a different residue of the same component protein or proteins, or an equal amount of tyrosine or threonine phosphorylation on the same component protein or proteins.

It is understood that, where there is a quantitative difference between the altered and control modification states, the difference does not fall within the inherent variability of the assay. In general, where the altered modification state is increased or decreased as compared to the control modification state, the altered modification state is increased or decreased by 50% or more as compared to the control modification state. An altered modification state also can represent an increase or decrease of 100% or more, or an increase or decrease of 2-fold, 3-fold, 4-fold, 5-fold, 10-fold, 20-fold or more relative to the control modification state.

An altered modification state can be determined for the receptor-containing complex as a whole, or can be determined for one or more individual components of the complex such as the nuclear hormone receptor, a heterodimeric partner of the nuclear hormone receptor or another associated protein such as a kinase or a 160 kDa protein. It is understood that an altered modification state of any one component of the receptor-containing complex serves to identify an “effective agent,” regardless of whether there is an altered modification state of any other components of the isolated receptor-containing complex.

As used herein, the term “control modification state” means the same post-translational modification of the same one or more components occurring in an isolated receptor-containing complex, where the nuclear hormone receptor contained in the isolated complex has not been contacted with the one or more agents.

A control modification state is determined using a cell sample such as a eukaryotic cell sample that is not contacted with the one or more agents to be assayed. For convenience, a eukaryotic cell sample that is not contacted with the one or more agents is designated herein the “control eukaryotic cell sample;” a eukaryotic cell sample contacted with the one or more agents is referred to herein as a “test eukaryotic cell sample.” The control eukaryotic cell sample can be the same sample as the test eukaryotic cell sample, provided that the control modification state is determined prior to contacting the test eukaryotic cell sample with the one or more agents. The control eukaryotic cell sample also can be different from the test eukaryotic cell sample; the control eukaryotic cell sample can be contacted, if desired, with vehicle or with reference agent having a known effect on the modification state to be assayed.

To determine a control modification state in a control eukaryotic cell sample different from the test eukaryotic cell sample, one skilled in the art would use a corresponding cell or tissue type and would culture the cells or tissue under the same conditions as the cells or tissue from which the test eukaryotic cell sample is prepared. Such a corresponding cell or tissue type preferably has the same amount and type of nuclear hormone receptor and the same amount and type of heterodimeric partner, and, most preferably, is of the identical cell or tissue type used to prepare the test eukaryotic cell sample. Even more preferably, cells or tissue of the identical cell or tissue type are grown under the same conditions as the cells or tissue from which the eukaryotic cell sample is prepared. As disclosed herein, for example, HEK 293 cells were transiently transfected with FLAG-RXRα and contacted with known RXR-specific agonists; as a control, the same transiently transfected HEK 293 cells were treated with control vehicle (see Example II). One skilled in the art understands that transiently transfected FLAG-RXRα HEK 293 cells or another immortalized human kidney cell line expressing a similar level of RXRα can be used as a control eukaryotic cell sample, where the test eukaryotic cell sample is FLAG-RXRα transfected HEK 293 cells. One skilled in the art further understands that a control modification state can be determined empirically before, after, or simultaneously with an assay performed to determine an altered modification state or can be determined, if desired, by referencing a historical value.

As disclosed herein, an altered modification state can occur rapidly upon treatment with one or more agents. As disclosed herein, RXRα is associated with a protein kinase in cells treated with RXR-specific ligand for only 10 minutes. Thus, in one embodiment of the invention, a nuclear hormone receptor is contacted with the one or more agents to be screened and conditions suitable for the “modification” are provided for a limited time. Typically, conditions suitable for the modification are provided for less than 2 hours following the contacting step, and can be provided for less than 1 hour, 45 minutes, 30 minutes, 25 minutes, 20 minutes, 10 minutes or 5 minutes following the contacting step. It is understood that, even while conditions suitable for the modification are limited in duration, for example, to less than 20 minutes following the contacting step, one skilled in the art can assay for an altered modification state at a much later time, for example 24, 48 or 72 hours later using, for example, frozen cell lysate. Thus, if desired, conditions suitable for the modification can be limited in duration to a brief time period, while the assay for the altered modification is performed after this time period.

In a method of the invention, an altered modification state occurs in an isolated receptor-containing complex. Thus, the isolated receptor-containing complex contains each enzyme, cofactor, protein or other molecule required for the altered modification. For example, where the altered modification state is an altered phosphorylation state, the receptor-containing complex can contain a kinase or phosphatase which is associated, directly or indirectly, with the receptor in the receptor-containing complex.

To distinguish from altered modification states occurring, for example, prior to isolation of the receptor-containing complex, conditions suitable for modification of the receptor-containing complex are provided after the complex has been isolated. For example, where the altered modification state is an increased phosphorylation state, conditions suitable for kinase activity are provided to the isolated receptor containing complex. Such conditions include, for example, a suitable magnesium concentration, which can be a concentration of about 1 mM to 25 mM. Other appropriate conditions can be, for example, an appropriate concentration of manganese, zinc or calcium.

The methods of the invention rely on a nuclear hormone receptor, which can be endogenous or exogenous, transiently or stably introduced into cultured cells, or provided as a purified or partially purified protein, for example, protein recombinantly expressed and purified from host cells such as bacterial, insect or mammalian cells, as described further below. A variety of nuclear hormone receptors can be useful in the methods of the invention including, for example, a retinoid X receptor (RXR), hepatocyte nuclear factor 4 (HNF4), testicular receptor, tailless gene homolog (TLX), chicken ovalbumin upstream promoter transcription factor (COUP-TF), thyroid receptor (TR), retinoic acid receptor (RAR), peroxisome proliferator activated receptor (PPAR), reverse Erb (revErb), RAR-related orphan receptor (ROR), steroidogenic factor-1 (SF-1), liver receptor homolog-1 (LRH-1), liver X receptor (LXR), farnesoid X receptor (FXR), vitamin D receptor (VDR), ecdysone receptor (EcR), pregnane X receptor (PXR), constitutive androstane receptor (CAR), neuron-derived activated receptor (NOR1), nuclear receptor related 1 (NURR1), estrogen receptor (ER), estrogen-related receptor (ERR), glucocorticoid receptor (GR), androgen receptor (AR), progesterone receptor (PR) or mineralocorticoid receptor (MR). In one embodiment, a nuclear hormone receptor useful in the invention is any nuclear hormone receptor except a thyroid hormone receptor.

In a further embodiment, a nuclear hormone receptor used in a method of the invention binds as a homodimer to its cognate response element. Such a nuclear hormone receptor can be, for example, a glucocorticoid, estrogen, androgen, progestin, or mineralocorticoid receptor. In yet a further embodiment, a nuclear hormone receptor used in a method of the invention binds as a heterodimer to its cognate response element. Such a nuclear hormone receptor can be, for example, a retinoic acid receptor, thyroid receptor, vitamin D receptor, farnesoid X receptor, oxysterol receptor, peroxisome proliferator receptor or ecdysone receptor, each of which bind as a heterodimer with the retinoid X receptor.

In a further embodiment of the invention, the nuclear hormone receptor is a retinoid X receptor, retinoic acid receptor, progesterone receptor, estrogen receptor, androgen receptor or vitamin D receptor. In yet another embodiment of the invention, the nuclear hormone receptor is a retinoid X receptor such as RXRα, RXRβ or RXRγ.

In its native form, a nuclear hormone receptor is a polypeptide which (1) contains a DNA-binding domain; (2) contains a ligand-binding domain; and (3) is localized in its naturally occurring environment, at least in part, to the nucleus of eukaryotic cells. A native nuclear hormone receptor generally has a DNA-binding domain containing two (Cys)₄ zinc finger motifs, and most often is a ligand-dependent transcription factor, for example, a ligand-dependent transcriptional activator. It is recognized that a nuclear hormone receptor may reside in the cytoplasm in the absence of ligand, translocating at least in part to the nucleus or other cellular compartment upon ligand-binding as in the case of the glucocorticoid and mineralocorticoid receptors. Thus, nuclear localization of a nuclear hormone receptor can be ligand-dependent. Nuclear hormone receptors useful in the invention include full length steroid hormone receptors; thyroid/retinoid/vitamin D and peroxisome proliferator activated receptors; and orphan receptors, and fragments of these receptors.

Native nuclear hormone receptors generally share a similar domain structure. An N-terminal extension of varying length often harbors a transactivation function (AF1), for example, in steroid receptors such as the estrogen and progesterone receptors. A well-conserved central DNA binding region typically contains two zinc-finger DNA binding motifs of the (Cys)₄ type. A variable C-terminal extension (CTE) flanks the zinc fingers and participates in DNA binding by some receptors, for example, thyroid receptor. A large C-terminal ligand binding domain (LBD) also is seen in nuclear hormone receptors, generally having ligand contacts in three distinct clusters and separate from receptor dimerization contacts that also occur in the ligand binding domain. The conserved E1 subregion, as well as a less well-conserved heptad nine (h9) region and a second transactivation domain (AF2) also lie within the ligand binding domain.

Native nuclear hormone receptors typically dimerize, either as a homodimer or as a heterodimer, for example, with RXR or USP, followed by high-affinity binding to specific hexanucleotide half-elements arranged in a particular motif. Many nuclear hormone receptors bind DNA in one of the following patterns: (1) as heterodimers with RXR (or USP) on directly (tandemly) repeated half elements separated by a spacer of 1-5 bp; (2) as heterodimers on inverted (palindromic) response elements separated by 1 bp; (3) as homodimers on direct repeats separated by 1 bp; (4) as homodimers on inverted repeats separated by 3 bp; or (5) as monomers on a single half-site, which may contain a 3 bp 5′ extension. The hexanucleotide half-element generally is a variation of AGGTCA, although several steroid receptors such as the glucocorticoid receptor, mineralocorticoid receptor, progesterone receptor and androgen receptor bind an AGAACA half-site. Exemplary heterodimers include RXR/RAR; RXR/VDR; RXR/LXR; RXR/PXR; RXR/CAR and PPAR/RXR, each of which bind to direct repeats, and RXR/FXR and USP/EcR, each of which bind to inverted half-repeats. Exemplary homodimers include glucocorticoid, estrogen, androgen and mineralocorticoid receptor homodimers, each of which bind to palindromic repeats separated by 3 bp. While both receptors of a homodimer likely are liganded for activity, liganding of the primary receptor residing on the 3′ half-element generally is sufficient for activity of a heterodimer (Whitfield, supra, 1999).

A variety of nuclear hormone receptors are known in the art, and these receptors as well as fragments, fusion proteins and variants of naturally occurring receptors are useful in the invention as described further below. See, for example, Mangelsdorf et al., Cell 83:835-9 (1995); Enmark and Gustafsson, Mol. Endocrinol. 10:1293-1307 (1996); Kumar and Thompson, Steroids 64:310-319 (1999); Whitfield et al., supra, 1999; Laudet, J. Mol. Endocrin. 19:207-226 (1997); and Giguere, Endocrine Rev. 20:689-725 (1999). Nucleic acid sequences encoding human and other mammalian, vertebrate and non-vertebrate nuclear hormone receptors readily can be obtained from a variety of sources, for example, from databases such as GenBank. For example, a nucleic acid sequence encoding human RXRα is available as GenBank accession NM_{—}002957; a nucleic acid sequence encoding human RXRβ is available as GenBank accession AF065396; and a nucleic acid sequence encoding human RXRγ is available, for example, as GenBank accession NM_{—}006917. Similarly, a nucleic acid sequence encoding human RARα is available as GenBank accession AF088890; a nucleic acid sequence encoding human RARβ is available as GenBank accession NM_{—}000965; and a nucleic acid sequence encoding human RARγ is available, for example, as GenBank accession M38258. Nucleic acid sequences encoding a variety of additional nuclear hormone receptors also are known in the art and readily available to the skilled person; sources for exemplary nucleic acid sequences useful in the invention are provided in Table 1. These sources and sequences are hereby incorporated by reference herein.

TABLE 1

| | GenBank |
|---|---|
| Nuclear Hormone Receptor | accession |
| human retinoid X receptor α (hRXRα) | NM_002957 |
| human retinoid X receptor β (hRXRβ) | AF065396 |
| mouse retinoid X receptor β (mRXRβ) | D21830 |
| human retinoid X receptor γ (hRXRγ) | NM_006917 |
| mouse retinoid X receptor γ (mRXRγ) | S62948 |
| jellyfish retinoid X receptor | AF091121 |
| human ovalbumin upstream promoter | NM_005654 |
| transcription factor | |
| human thyroid receptor α (hTRα) | X55070 |
| mouse thyroid receptor β (mTRβ) | U15544 |
| Xenopus thyroid receptor β | U04675 |
| Fugu fish thyroid receptor α | AJ012380 |
| human retinoic acid receptor α (hRARα) | AF088890 |
| human retinoic acid receptor β (hRARβ ) | NM_000965 |
| human retinoic acid receptor γ (hRARγ) | M38258 |
| human peroxisome proliferator activated | AB005523 |
| receptor γ (hPPARγ) | |
| mouse RAR-related orphan receptor γ | AF019657 |
| human steroidogenic factor-1 (hSF-1) | S65878 |
| human vitamin D receptor (hVDR) | AB002162 |
| Drosophila ecdysone receptor | M74078 |
| mouse constitutive androstane receptor α | AF009326 |
| human nuclear receptor related 1 | AB019433 |
| (hNURR1) | |
| mouse nuclear receptor related 1 | AAC53153 |
| (mNURR1) | |
| human estrogen receptor α (hERα) | AF123494 |
| Atlantic salmon estrogen receptor α | AF047895 |
| tilapia fish estrogen receptor α | X93558 |
| human glucocorticoid receptor (hGR) | U78508 |
| human androgen receptor (hAR) | M27423 |
| human progesterone receptor (hPR) | X69071 |
| chicken progesterone receptor (cPR) | M32726 |
| human mineralocorticoid receptor (hMR) | AF068617 |

As used herein, the term “nuclear hormone receptor” means a polypeptide containing the ligand binding domain of a nuclear hormone receptor. Such a nuclear hormone receptor retains the ability to bind a known ligand of one of the nuclear hormone receptors referenced in Table 1, or contains a ligand binding domain exhibiting primary, secondary or tertiary structural homology to one of the ligand-binding domains of the nuclear hormone receptors referenced in Table 1, or both. Where a nuclear hormone receptor is one of the receptors shown in Table 1, the receptor retains the ability to bind a known ligand with a binding constant (K_{d}) of at least 300 nM, and can bind, for example, with a K_{d} of at least 200 nM, 100 nM, 75 nM, 50 nM, or higher.

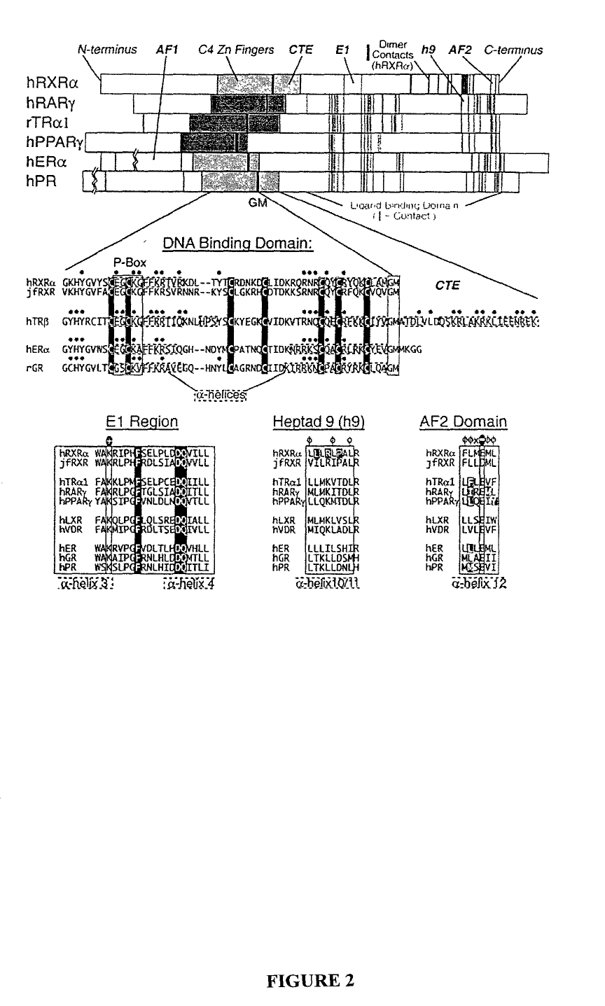

Within its ligand binding domain, a nuclear hormone receptor can exhibit primary, secondary or tertiary structural homology to at least one of the ligand-binding domains of the nuclear hormone receptors referenced in Table 1 and generally has a tertiary structure which is a sandwich of 11 to 13 α-helices and several small β-strands organized around a lipophilic binding cavity (Williams and Sigler, Nature 393:392-396 (1998)). A nuclear hormone receptor ligand-binding domain generally contains three subregions: a conserved El domain; the heptad 9 (h9) subregion; and an AF2 subregion (Whitfield et al., supra, 1999). A nuclear hormone receptor can be recognized, for example, as a protein containing the conserved lysine, phenylalanine and aspartic acid-glutamine residues in the E1 subregion as shown in FIG. 2.

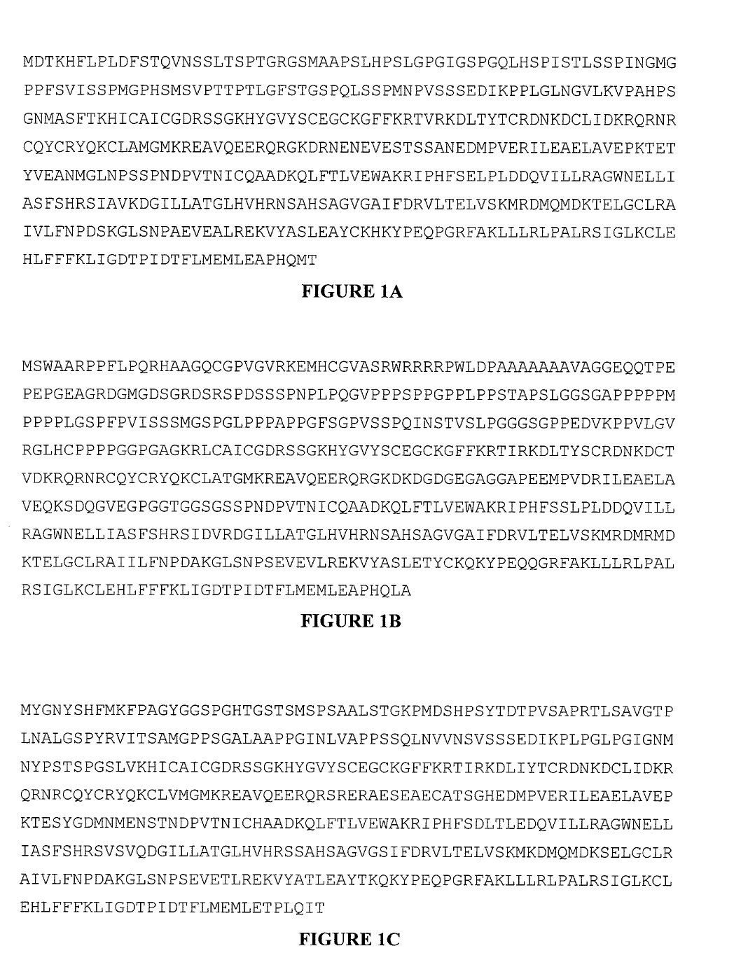

The term nuclear hormone receptor encompasses polypeptides having an amino acid sequence that is identical to the wild type hormone receptor sequence, and polypeptides having a similar, non-identical sequence that is considered by those skilled in the art to be a functionally equivalent amino acid sequence. An RXR receptor, for example can have an amino acid sequence identical to one of the RXR isoforms (SEQ ID NOS: 1, 2 and 3) shown in FIG. 1, or a similar, non-identical sequence that is functionally equivalent.

It is understood that limited modifications can be made without destroying the biological function of a nuclear hormone receptor useful in the invention. Minor modifications of human RXRα (SEQ ID NO: 1) that do not destroy ligand binding activity fall within the definition of RXR. Similarly, minor modifications of human retinoic acid receptor a that do not destroy ligand binding fall within the definition of retinoic acid receptor α, and minor modifications of human estrogen receptor that do not destroy estrogen binding activity fall within the definition of an estrogen receptor.

It is understood that minor modifications of primary amino acid sequence can result in polypeptides which have substantially equivalent or enhanced function as compared to wild type sequences, for example, the human RXRα, β and γ sequences set forth in FIG. 1. These modifications can be deliberate, as through site-directed mutagenesis, or can be accidental such as through mutation in hosts harboring an encoding nucleic acid. All such modified polypeptides are included in the definition of the particular nuclear hormone receptor as long as ligand binding activity or primary, secondary or tertiary structural homology to one of the receptors shown in Table 1 are retained. It further is understood that various moieties can be attached to a nuclear hormone receptor, for example, a retinoid X receptor, retinoic acid receptor, progesterone receptor, estrogen receptor, androgen receptor or vitamin D receptor; such moieties include other polypeptides, carbohydrates, lipids, or chemical moieties. These fusion polypeptides or polypeptide conjugates also can be used in the screening methods of the invention.

Retinoids exert their biological effects through one or both of two families of nuclear hormone receptors, retinoic acid receptors (RARs) and retinoid X receptors (RXRs). Retinoic acid receptors and retinoid X receptors are ligand-dependent transcription factors which regulate gene transcription by both upregulating gene expression through binding RA-responsive elements and down-regulating gene expression by antagonizing the enhancer action of other transcription factors such as AP1. Distinct RXRα, RXRβ and RXRγ isotypes and RARα, RARβ and RARγ isotypes are encoded by separate genes. Both RXR and RAR isotypes can be further expressed as several isoforms. RAR isoforms differ in the N-terminal A region; these isoforms are generated by alternative splicing or differential usage of two promoters. Like other nuclear hormone receptors, in their native form RAR and RXR receptors contain AF1, DNA-binding, and ligand binding domains (see above). While RXR homodimers are responsive to RXR-activating compounds, the RXR subunit can be a silent partner in some heterodimers; for example, synthetic RXR agonists do not activate the RAR/RXR heterodimer.

All trans-retinoic acid is the physiological hormone for the RAR receptors and does not bind the RXR receptors. 9-cis-retinoic acid, an RXR receptor ligand, also binds to the RAR receptors. Various RXR and RAR specific synthetic ligands have been synthesized. For example, LG 100268, AGN 192599, SR 11217, and SR 11237 are RXR specific synthetic retinoids binding to all three RXRs but not to any of the RAR isotypes. TTNPB is an RAR-specific synthetic retinoid that binds RARs but not RXRs. RAR-selective ligands include AGN 190299, a RARβ/γ selective ligand, and Am 580 and Am 80, which are RARα-selective in in vitro binding assays. Thus, RXR and RAR ligands can be selective or non-selective and can be naturally occurring or synthetic.

A method of the invention can be advantageously practiced with a retinoid X receptor. The term “retinoid X receptor” is synonymous with “RXR” and means a polypeptide which contains a ligand binding domain that binds a known retinoid X receptor ligand, for example, LG 100268, AGN 192599, SR 11217, or SR 11237. A retinoid X receptor can have the ligand binding domain of one of the naturally occurring human RXR isoforms such as human RXRα (SEQ ID NO:1), human RXRβ (SEQ ID NO: 2) or human RXRγ (SEQ ID NO: 3) shown in FIG. 1 and is intended to include related polypeptides having a ligand binding domain with primary, secondary or tertiary structural homology to the ligand binding domain of one of the RXR isoforms provided herein as SEQ ID NOS: 1, 2 or 3. Such related polypeptides exhibit greater structural similarity to the amino acid sequence of RXRα, RXRβ or RXRγ than to other nuclear hormone receptors containing (Cys)₄-type zinc finger motifs and include alternatively spliced forms of human RXRα, RXRβ or RXRγ; species homologs including mouse, rat, primate and other mammalian homologs, vertebrate homologs and non-vertebrate homologs; and isotype variants of the amino acid sequences shown in FIG. 1, provided that the polypeptide retains the ability to bind a known retinoid X receptor ligand such as LG 100268, AGN192599, SR 11217, or SR 11237. In one embodiment, a retinoid X receptor contains a ligand binding domain having at least 75% amino acid identity with the ligand binding domain of SEQ ID NO: 1. In other embodiments, a retinoid X receptor contains a ligand binding domain having at least 80%, 85%, 90% or 95% amino acid identity with the ligand binding domain of SEQ ID NO: 1. A retinoid X receptor typically binds a known retinoid X receptor ligand with a binding constant (K_{d}) of at least 300 nM, and can bind, for example, with a K_{d} of at least 200 nM, 100 nM, 75 nM, 50 nM, or higher.

The term nuclear hormone receptor, as used herein, also encompasses variants of wild type nuclear hormone receptors, for example, truncated nuclear hormone receptors, receptors containing one or more point mutations, or fusion proteins containing one or more heterologous hormone receptor or non-hormone receptor sequences in addition to the primary nuclear hormone receptor sequence. In one embodiment, a nuclear hormone receptor used in a screening method of the invention is a truncated nuclear hormone receptor lacking a native DNA-binding domain. Such a truncated receptor can be, for example, a nuclear hormone receptor consisting essentially of the ligand-binding domain.

As used herein, the term “truncated nuclear hormone receptor” is synonymous with “truncated receptor” and means a deletion derivation of a wild type nuclear hormone receptor that lacks a portion of the wild type nuclear hormone receptor polypeptide sequence. A truncated nuclear hormone receptor can contain an N-terminal, internal or C-terminal deletion, or a combination thereof, and generally lacks 20 or more contiguous amino acids as compared to the wild type hormone receptor. A truncated nuclear hormone receptor can have a deletion of, for example, 50 or more, 100 or more, 150 or more, 200 or more, 250 or more, or 300 or more amino acids as compared to wild type hormone receptor.

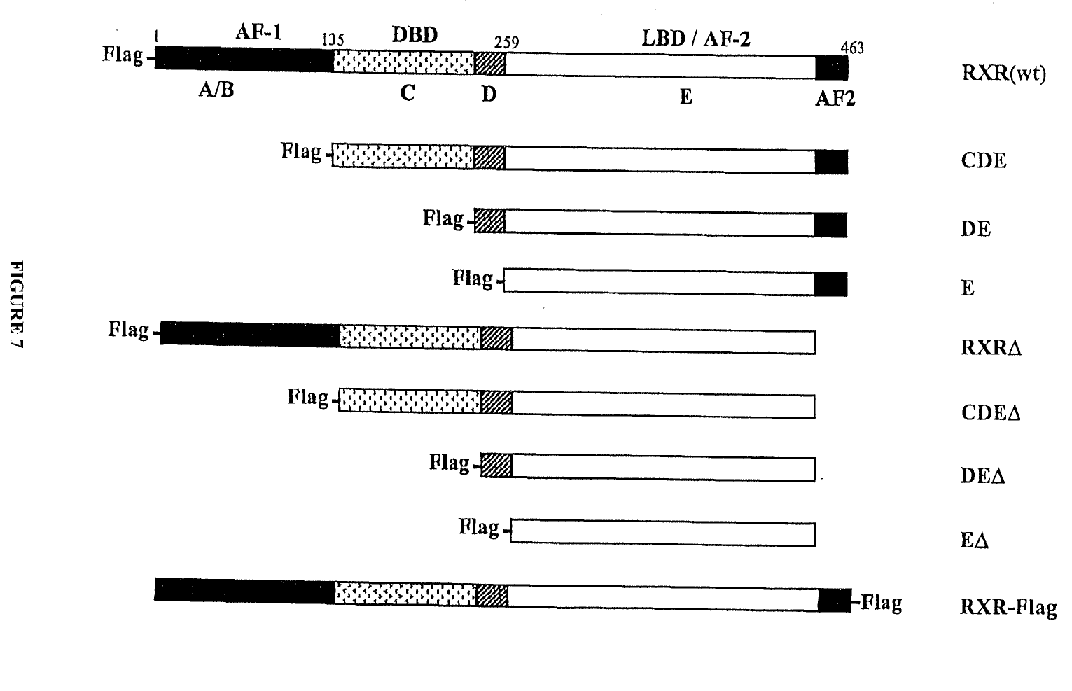

As disclosed herein, a truncated human RXRα receptor containing only the ligand-binding domain (RXRαE containing residues 259 to 463) recruited kinase activity to an immunocomplex in the presence of RXR-specific agonist (Example IIIB and FIG. 8). Thus, a screening method of the invention can be practiced, for example, using a truncated nuclear hormone receptor consisting essentially of the ligand-binding domain. Such a receptor contains a functional ligand-binding domain and may contain various flanking residues adjacent to this domain but does not any other complete or functional nuclear hormone receptor domains such as the transactivation domain “A/B” or DNA-binding domain “C.” A ligand-binding domain of a nuclear hormone receptor generally is characterized, in part, as a sandwich of 11 to 13 α-helices and several small β-strands organized around a lipophilic binding cavity (Williams and Sigler, supra, 1998). A ligand-binding domain of a nuclear hormone receptor also contains three subregions: the conserved E1 domain, which includes conserved phenylalanine and aspartic acid-glutamine residues and can participate in dimerization and transactivation; the heptad 9 (h9) subregion, which can mediate, in part, dimerization; and the AF2 subregion, which can contain ligand contacts and effect transactivation (Whitfield et al., supra, 1999). Thus, it is recognized that a nuclear hormone receptor consisting essentially of a ligand-binding domain contains, in part, the AF2 subregion. It is understood that a truncated nuclear hormone receptor consisting essentially of the ligand-binding domain can be fused to one or more heterologous sequences for use in a screening method of the invention.

A receptor consisting essentially of a ligand-binding domain contains a ligand binding domain and may contain various nuclear hormone receptor flanking residues adjacent to this domain but does not contain any other complete or functional nuclear hormone receptor domains such as the transactivation domain “A/B” or the DNA-binding domain “C.” A variety of truncated nuclear hormone receptors consisting essentially of the ligand-binding domain can be used in a method of the invention. The ligand-binding domain of human RXRα is provided herein as residues 259 to 463 of SEQ ID NO: 1. Additional nuclear hormone receptor ligand-binding domains are well known in the art; for example, residues 229 to 387 of GenBank accession XM_{—}008647 encodes a human RARα ligand-binding domain; residues 719 to 829 of GenBank accession XM_{—}006190 encodes a human progesterone receptor ligand-binding domain; and 231 to 393 of GenBank accession NM_{—}000376 encodes a human vitamin D receptor ligand-binding domain. It is understood that these and additional nuclear hormone receptor ligand-binding domains are known in the art, or can be determined by comparison to known ligand-binding domains.

A nuclear hormone receptor useful in the invention also can be a variant with an increased ratio of cytoplasmic to nuclear localization as compared to wild type nuclear hormone receptor, or a variant that lacks a functional DNA-binding domain.

As disclosed herein, a human RXRα receptor lacking any functional DNA-binding domain was effective in recruiting kinase to the RXRα-containing immunocomplex when treated with RXR-specific ligand, AGN194204 (see Example IIIB and FIG. 8). These results demonstrate that DNA-binding activity is not necessary for phosphorylation of RXRα or the associated 160 kDa protein and indicate that DNA-binding activity is not necessary for altered ligand-dependent modification of a nuclear hormone receptor. Thus, a method of the invention can be practiced with a nuclear hormone receptor variant that lacks a functional DNA-binding domain. Such a variant lacks the capacity to specifically bind DNA and can contain, for example, one or more point mutations such as one or more amino acid additions, deletions or insertions relative to the wild type nuclear hormone receptor, or can be, for example, a truncated receptor lacking part or all of the DNA-binding domain. Where the wild type nuclear hormone receptor contains (Cys)₄ zinc fingers, a variant can contain, for example, an amino acid substitution at one of the conserved cysteine residues. Specific DNA-binding activity readily can be determined using the cognate DNA-binding site and in vitro methods well known in the art, including gel shift and DNase I footprint assays.

A nuclear hormone receptor for use in a screening method of the invention can be a fusion protein containing a heterologous peptide or polypeptide sequence from a different nuclear hormone receptor, or from a protein that is not a nuclear hormone receptor. Such a fusion protein can contain, for example, a heterologous membrane-anchoring domain, heterologous epitope tag, heterologous protein kinase recognition sequence, or any combination of these heterologous sequences, in addition to the nuclear hormone receptor or truncated portion thereof.

The term “heterologous,” as used herein in reference, for example, to a membrane-anchoring domain, epitope tag or protein kinase recognition sequence, means a domain, tag or sequence derived from a different gene than the gene encoding the fused nuclear hormone receptor. Thus, for example, in a FLAG-RXRα fusion protein, the “FLAG” tag is a heterologous epitope tag, which is not found in the gene encoding RXRα.

In one embodiment, a nuclear hormone receptor is expressed as a fusion protein containing a heterologous membrane-anchoring domain. Such a fusion protein can have an increased ratio of cytoplasmic to nuclear localization as compared to the same native unfused nuclear hormone receptor. As used herein, the term “membrane-anchoring domain” means a peptide or polypeptide fragment that functions to direct a linked protein to the cell cytoplasmic membrane. Such a membrane-anchoring domain can be an naturally or non-naturally occurring sequence. A membrane-anchoring domain can be a naturally occurring sequence present, for example, in a membrane-associated protein such as a Src family tyrosine kinase, an insulin receptor substrate, phospholipase C, protein kinase B or C, or a PI3 kinase. A membrane-anchoring domain useful in the invention also can be, for example, a myristoylation (MYR) domain (Resh, Biochim. Biophys. Acta 1451:1-6 (1999)) from, for example, a Src family kinase; a pleckstrin homology (PH) domain derived, for example, from an insulin receptor substrate, phospholipase C (PLC) or protein kinase B (PKB; Lemmon et al., Cell 85:621-624 (1996)); or a C2 domain derived, for example, from protein kinase C (PKC; Newton and Johnson, Biochim. Biophys. Acta 1376:155-172 (1998)) or a PI3 kinase. The skilled artisan understands that these and other heterologous membrane-anchoring domains can be fused to a nuclear hormone receptor and can amplify the difference between the altered modification state and the control modification state.

In another embodiment, a hormone receptor is expressed as a fusion protein containing a heterologous epitope tag, which can provide a convenient means for isolating the receptor-containing complex. A variety of heterologous epitope tags are well known and routine in the art including FLAG, hemagluttinin (HA), c-myc, 6-HIS and AU1 tags. The FLAG tag DYKDDDDK (SEQ ID NO: 39), for example, can be used as an epitope tag (see Chubet and Brizzard, BioTechniques 20:136-141 (1996)). Well known heterologous epitope tags include the HA tag YPYDVPDYA (SEQ ID NO: 40); the c-Myc epitope EQKLISEEDL (SEQ ID NO: 41); the AU1 tag DTYRYI (SEQ ID NO: 42); and the 6-HIS tag HHHHHH (SEQ ID NO: 43). One skilled in the art understands that these and other heterologous epitope tags can be fused to a nuclear hormone receptor for use in a method of the invention.

Where the altered modification state is an altered phosphorylation state, engineered protein kinase recognition sequences can be used to “amplify” the signal of the screening assay, i.e. can increase the difference between the altered phosphorylation state and the control phosphorylation state. Thus, a screening method of the invention can be practiced with fusion protein that contains a nuclear hormone receptor fused to a heterologous protein kinase recognition sequence, which can be a synthetic or naturally occurring sequence. A heterologous protein kinase recognition sequence can occur naturally in, for example, a component of the receptor-containing complex such as a 160 kDa protein. The invention also can be practiced with a nuclear hormone receptor engineered to contain one or more protein kinase recognition motifs that represent duplicated sequences occurring naturally in the nuclear hormone receptor.

A protein kinase recognition motif can contain, for example, a contiguous sequence of six to ten residues of a nuclear hormone receptor, where the contiguous sequence includes a serine or threonine residue. In one embodiment, a protein kinase recognition motif contains six to ten contiguous residues of a RXR receptor “A/B” domain, where the contiguous sequence includes a serine or threonine residue. For example, a protein kinase recognition motif can have six to ten contiguous residues of residues 1 to 134 of human RXRα (SEQ ID NO: 1), where the contiguous sequence includes a serine or threonine residue. In one embodiment, a protein kinase recognition motif contains six to ten contiguous residues of human RXRα (SEQ ID NO: 1) and includes serine-56, serine-70 or threonine-82. In another embodiment, a protein kinase recognition motif contains six to ten contiguous residues of human RXRα (SEQ ID NO: 1) and includes serine-260. One skilled in the art understands that, in other retinoic acid receptor species homologs and isoforms, analogous protein kinase recognition motifs can be produced.

A variety of heterologous protein kinase recognition sequences are well known in the art and typically are short sequences of less than 10 residues. Several types of protein kinase recognition sequence are recognized, for example, by serine/threonine kinases. A serine/threonine kinase preferring arginine/lysine/histidine near the catalytic site Ser/Thr is designated a positive charge directed kinase; a protein kinase preferring acidic or phospho-amino acids is designated a negative-charge directed protein kinase; and a kinase recognizing a Ser/Thr-Pro motif is designated a proline-directed kinase. Positive charge directed kinases can recognize, for example, the sequence RRXSI (SEQ ID NO: 44) or RRKXSFK(F/L)XRQXSF (SEQ ID NO: 45); negative-charge directed kinases can recognize, for example, S^{P}XXSI, DEESEED (SEQ ID NO: 46), or SXXXS^{P}; and proline directed kinases can recognize, for example, (R/K)SPX(R/K) (SEQ ID NO: 47) or PXSP, where X is any amino acid and S^{P} is phosphoserine (Songyang, Progress Biophys. & Molec. Biol. 71:359-372 (1999)). The introduction of heterologous protein kinase recognition sequences is well known in the art, as described, for example, in Pestka et al., Protein Exp. Purif. 17:203-214 (1999); Johnson et al., FEBS Lett. 430:1-11 (1998); and Songyang, supra, 1999.

A heterologous protein kinase recognition sequence also can be phosphorylated by a tyrosine kinase. Such a recognition sequence generally contains acidic amino acids such as Asp or Glu at the N-terminus of the tyrosine residue (positions Y^{31 2}, Y^{31 3} and Y^{31 4}) and, in addition, can contain Ile/Leu/Val at the Y^{31 1} position and acidic or small amino acids at the Y⁺¹ position, if the tyrosine kinase is a cytosolic enzyme (Songyang, supra, 1999). Exemplary tyrosine kinase recognition sequences include EEEIYEEIE (SEQ ID NO: 48); DEEIYE/GEFF (SEQ ID NO: 49); XXVIYAAPF (SEQ ID NO: 50) and XEXIYGVLF (SEQ ID NO: 51; Songyang, supra, 1999). A variety of serine/threonine and tyrosine protein kinase recognition sequences are well known in the art as described, for example, in Kemp and Pearson, Trends Biochem. Sci. 15:342-346 (1990).

In addition to practicing the methods of the invention with a nuclear hormone receptor, these methods can be practiced with a member of the steroid hormone receptor superfamily. The steroid hormone receptor superfamily includes the nuclear hormone receptors and additionally encompasses non-nuclear receptors which may or may not have a DNA-binding domain. Members of the steroid hormone receptor superfamily contain a ligand-binding domain with primary, secondary or tertiary structural homology to the ligand-binding domain of nuclear hormone receptors and generally have a tertiary structure which is a sandwich of 11 to 13 α-helices and several small β-strands organized around a lipophilic binding cavity (Williams and Sigler, Nature 393:392-396 (1998)). A ligand-binding domain of a steroid hormone receptor superfamily member generally contains three subregions: a conserved E1 domain; the heptad 9 (h9) subregion; and an AF2 subregion (Whitfield et al., supra, 1999). A member of the steroid hormone receptor superfamily generally is characterized, in part, by containing conserved lysine, phenylalanine and aspartic acid-glutamine residues in the E1 subregion as shown in FIG. 2. In addition, a polypeptide that binds a nuclear hormone receptor ligand or a ligand of a member of the steroid hormone receptor superfamily is, itself, a member of the steroid hormone receptor superfamily.

In a method of the invention, a nuclear hormone receptor is contacted with one or more agents. As used herein, the term “agent” means any organic molecule, for example, a small molecule chemical; a peptide, peptidomimetic or peptoid; a protein, which can be an antibody or antigen-binding fragment thereof or a non-antibody protein; a nucleic acid molecule, for example, an oligonucleotide; an oligosaccharide; a lipoprotein; a glycolipid; or a lipid. Both naturally occurring and synthetic agents can be screened in a method of the invention. Naturally occurring agents are a product of nature in that the groups making up the molecule and the bonds linking the groups are produced by normal metabolic processes.

Agents to be screened generally are small lipophilic molecules that can diffuse across the plasma membrane and into cells freely. These molecules can be, for example, naturally occurring or synthetic retinoids (analogs of retinoic acid), eicosanoids, steroids, terpene-derived molecules and amino acid derivatives.

If desired, a population of agents can be assayed for activity en masse or in pools. For example, to identify an effective agent that modulates a biological activity of RARα, HEK 293 cells transfected with a RXRα-encoding nucleic acid molecule can be contacted with a population of agents and assayed for the ability to produce increased phosphorylation of an isolated RXRα-containing complex; the active population can be subdivided and the assay repeated in order to isolate the effective agent from the population. Such screening protocols, in which compounds are assayed in pools of 10, 50, 100, 200, 500, 1000 or 10,000, for example, are well within the ability of those skilled in high throughput and ultra high throughput screening technology.

The methods of the invention rely on assaying for an altered modification state occurring in an isolated receptor-containing complex to identify an effective agent that modulates a biological activity of the nuclear hormone receptor of interest. One skilled in the art understands that an “effective agent” that modulates a biological activity of a nuclear hormone receptor can reduce, enhance or change a biological activity of the nuclear hormone receptor either directly or indirectly and can be, for example, a precursor of an active compound, or a ligand of the nuclear hormone receptor.

To identify an effective agent according to a method of the invention, a nuclear hormone receptor is contacted with one or more agents. As used herein, the term “contacting” encompasses addition of the one or more agents to a lysate; addition of the one or more agents to a culture dish, flask or microtiter plate; and oral administration, injection, microinjection, infusion, or implantation of a slow release medium containing the one or more agents to be tested into an animal. Concentrations of agents to be tested generally are in the 10⁻¹² to 10⁻⁵ molar range and can be, for example, in the 10⁻⁹ to 10⁻⁶ molar range.

A method of the invention can be practiced with a variety of eukaryotic cell samples, including viable cells, which can be, for example, transiently or stably transfected cell; a whole cell lysate; or a fractionated cell lysate. A variety of eukaryotic cells are useful in the methods of the invention, including primary and immortalized cells, and a variety of cell types such as fibroblasts and adipocytes. A eukaryotic cell sample also can be prepared from a tumor cell, for example, a melanoma, colon tumor, breast tumor, prostate tumor, glioblastoma, renal carcinoma, neuroblastoma, lung cancer, bladder carcinoma, plasmacytoma or lymphoma cell. Where the nuclear hormone receptor is an RAR receptor, RXR receptor, or combination thereof, convenient cell types are, for example, the human embryonic kidney cell line HEK293, the human cell line HeLa and the green monkey cell line CV-1. The test sample containing the nuclear hormone receptor and one or more agents to be assayed also can include, if desired, an exogenous heterodimeric partner of the nuclear hormone receptor, or an exogenous kinase that enhances detection of an altered phosphorylation state. In one embodiment, the receptor-containing complex includes a serine/threonine kinase.

A eukaryotic cell sample useful in the invention can be prepared from transiently or stably transfected cells, or from an animal expressing an exogenous nuclear hormone receptor. Methods for stably or transiently introducing a vector or nucleic acid molecule into a eukaryotic cell are well known in the art and include calcium phosphate transfection, electroporation, microinjection, DEAE-dextran and lipofection methods (see, for example, Ausubel et al., Current Protocols in Molecular Biology John Wiley & Sons, Inc. New York (2000)). A viral vector also can be useful to express an exogenous nuclear hormone receptor in a eukaryotic cell. Such a viral vector can be, for example, a retroviral vector, adenoviral vector, Herpes simplex virus vector, vaccinia virus vector, cytomegalovirus vector, Moloney murine leukemia virus vector, lentivirus vector, adeno-associated virus vector, or the like.

Expression of a nucleic acid molecule encoding a nuclear hormone receptor in vivo can be carried out using one of numerous methods well known in the art including adenoviral transformation, retroviral transformation, ballistic gun delivery, lentiviral transformation, cytomegaloviral transformation, and microinjection.

Where a nuclear hormone receptor or member of the steroid hormone receptor superfamily, or a fragment thereof, is provided in purified or partially purified form, the receptor or fragment can be produced routinely using recombinant methods or by chemical or proteolytic cleavage of the isolated polypeptide. Methods for chemical and proteolytic cleavage and for purification of the resultant hormone receptors are well known in the art as described, for example, in Deutscher, Methods in Enzymology, Vol. 182, “Guide to Protein Purification,” San Diego: Academic Press, Inc. (1990).

A variety of means can be used to isolate the receptor-containing complex in a method of the invention. Isolation can be performed by specific binding to the receptor-containing complex, for example, by specific binding to the nuclear hormone receptor component of the complex. In one embodiment, a receptor-containing complex is isolated by immunoprecipitation.

A receptor-containing complex conveniently can be isolated by expressing the nuclear hormone receptor or other component of the receptor-containing complex as a fusion protein with a heterologous epitope tag. Convenient heterologous epitope tags include FLAG, hemagluttinin (HA), c-myc, 6-HIS and AU1 epitope tags. Fusion proteins containing the FLAG tag DYKDDDDK (SEQ ID NO: 39) can be produced by routine molecular methods; anti-Flag monoclonal antibodies are commercially available from, for example, Eastman Kodak (Rochester, N.Y.) and Berkeley Antibody Company (BabCO; Richmond, Calif.), and polyclonal serum is available from Santa Cruz Biotechnology (Santa Cruz, Calif.). The HA tag YPYDVPDYA (SEQ ID NO: 40) can be engineered into a recombinant nuclear hormone receptor or other component, and anti-HA antibody or antiserum obtained from BabCO, Roche Diagnostics (Indianopolis, Ind.) or Santa Cruz Biotechnology. Similarly, one can engineer the c-Myc epitope EQKLISEEDL (SEQ ID NO: 41), which is recognized by antibody or antisera commercially available from BabCO, Invitrogen (San Diego, Calif.), Roche Diagnostics, SIGMA (St. Louis, Mo.) or Santa Cruz Biotechnology. Addition epitope tags useful in the invention include the AU1 tag DTYRYI (SEQ ID NO: 42), which is recognized by a monoclonal antibody available from BabCO, and the 6-HIS tag HHHHHH (SEQ ID NO: 43), which is recognized by antibodies and antisera available, for example, from BabCO, Invitrogen, SIGMA or Santa Cruz Biotechnology. If desired, a fusion protein containing a 6-HIS epitope can be purified using metal chelate chromatography (see Ausubel et al., supra, 10.15, Supplement 41). One skilled in the art understands that these and other epitope tags can be conveniently used to isolate a receptor-containing complex in a method of the invention.

Immunoaffinity purification can be performed, for example, using antibody or antisera immunoreactive with an epitope of the nuclear hormone receptor; antibody or antisera immunoreactive with an epitope of a component of the receptor-containing complex, or can be performed using antibody or antisera immunoreactive with a heterologous epitope tag fused to a component of the receptor-containing complex. In one embodiment of the invention, a heterologous epitope tag is fused to the nuclear hormone receptor, and an antibody or antisera that is immunoreactive with the epitope tag is used to isolate the receptor-containing complex.

Affinity purification, including immunoaffinity, DNA affinity, and other types of affinity purification, can be used to isolate a receptor-containing complex. In one embodiment, a nuclear hormone receptor or other component of the receptor-containing complex is expressed as a fusion protein in a form suitable for affinity purification, for example, as a fusion with glutathione S transferase (GST). To produce such a fusion protein, a nuclear hormone receptor can be cloned into a pGEX vector (Amersham Pharmacia; Piscataway, N.J.) for expression as a C-terminal fusion protein with glutathione S transferase; expressed in bacteria; and subsequently purified using affinity to glutathione agarose (Ausubel, supra, 2000; Chapter 16 and Supplement 28). After contacting purified GST-receptor or a lysate containing the GST-receptor fusion protein with one or more agents and a eukaryotic cell sample, a receptor-containing complex can be isolated using affinity purification, for example, with glutathione-agarose (Ausubel, supra, 2000, Chapter 20 and Supplement 33).

Immunoprecipitation can be conveniently used to isolate a receptor-containing complex in a method of the invention. As used herein, the term “immunoprecipitation” means any process by which an antigen or antigen-containing complex is isolated by binding to a specific antibody attached to a sedimentable matrix. Immunoprecipitation is performed by addition of a specific antibody to a sample that includes the receptor-containing complex; the specific antibody can be polyclonal antisera, or one or more monoclonal antibodies, and is attached to a sedimentable matrix, which can be, for example, protein A or protein G-agarose beads, or Sepharose. It is recognized that the polyclonal or monoclonal antibody can specifically bind, for example, a native nuclear hormone receptor epitope; an epitope of a protein associated with the nuclear hormone receptor, such as an epitope on an associated 160 kDa protein or HSP90; or can specifically bind a heterologous epitope tag fused to the receptor or an associated protein. Low-speed centrifugation typically is performed to separate the solid-phase matrix and bound proteins, and washing is performed to remove unbound proteins. Exemplary conditions for immunoprecipitating a nuclear hormone receptor using an epitope tagged-hormone receptor fusion protein are disclosed herein in Example II, which discloses coimmunoprecipitation of RXRα and an associated kinase from HEK 293 cells transiently transfected with FLAG-RXRα using an anti-FLAG antibody. In addition, a variety of immunoprecipitation protocols are well known in the art, as described, for example, in Harlow and Lane, Antibodies: A Laboratory Manual (Cold Spring Harbor Laboratory Press, 1988); and Ausubel, supra, 2000 (see especially and Chapter 10 (Supplement 48) and Chapter 20 (Supplement 46)).

An antibody useful in immunoprecipitation or other immunoaffinity purification of a receptor-containing complex can be polyclonal or monoclonal, or a pool of monoclonal antibodies, and, furthermore, can be a polypeptide fragment of an antibody that retains a specific binding activity for a nuclear hormone receptor, associated protein, or heterologous epitope tag of at least about 1×10⁵ M⁻¹. One skilled in the art would know that antibody fragments such as Fab, F(ab′)₂ and Fv fragments can retain specific binding activity and, thus, can be useful in the invention. It further is understood that immunoprecipitation or other immunoaffinity purification can be performed with a non-naturally occurring antibody or fragment containing, at a minimum, one V_{H} and one V_{L} domain, for example, a chimeric antibody, humanized antibody or single chain Fv fragment (scFv) that specifically binds a nuclear hormone receptor or associated protein. Such a non-naturally occurring antibody can be constructed using solid phase peptide synthesis, produced recombinantly or obtained, for example, by screening combinatorial libraries consisting of variable heavy chains and variable light chains as described by Borrebaeck (Ed.), Antibody Engineering (Second edition) New York: Oxford University Press (1995)).

An antibody or antiserum useful in the invention can be obtained commercially or prepared by routine methods, for example, using a nuclear hormone receptor fusion protein or a synthetic peptide encoding a portion of a nuclear hormone receptor as an immunogen. One skilled in the art would know that purified nuclear hormone receptor, which can be produced recombinantly, or fragments of a nuclear hormone receptor, including peptide portions such as synthetic peptides, can be used as an immunogen. Non-immunogenic fragments or synthetic peptides of a nuclear hormone receptor can be made immunogenic by coupling the hapten to a carrier molecule such as bovine serum albumin (BSA) or keyhole limpet hemocyanin (KLH). In addition, various other carrier molecules and methods for coupling a hapten to a carrier molecule are well known in the art are described, for example, by Harlow and Lane, supra, 1988. Where the antibody binds a heterologous epitope, for example, a FLAG, hemagluttinin (HA), c-myc, or AU1 or 6-HIS tag, an antibody that specifically binds the epitope typically is commercially available, for example, from BabCO, Invitrogen (San Diego, Calif.), Roche Diagnostics, SIGMA (St. Louis, Mo.) or Santa Cruz Biotechnology, as described hereinabove.

In one embodiment of the invention, an isolated receptor-containing complex is assayed for an altered phosphorylation state occurring in the isolated complex as compared to a control phosphorylation state. A variety of routine techniques can be used to assay for such an altered phosphorylation state, including techniques that detect a change in the molecular weight, charge, hydrophilicity, solubility, antigenicity or binding properties of one or more components of the receptor-containing complex, or a change in the molecular weight, charge, hydrophilicity, solubility, antigenicity or binding properties of the isolated receptor-containing complex as a whole.

An altered phosphorylation state also can be determined using an antibody that specifically binds tyrosine phosphate; serine phosphate; or threonine phosphate. Unlabelled lysates can be electrophoresed and immunoblotted with anti-phosphotyrosine antibody, for example, to detect an altered tyrosine phosphorylation state. Numerous anti-phosphoserine, anti-phosphothreonine and anti-phosphotyrosine antibodies have been developed and are known in the art, including monoclonal anti-antiphosphoserine, anti-phosphothreonine and anti-phosphotyrosine antibodies commercially available from Sigma (Yan et al., J. Chromatography 808:23-41 (1998)). It is understood that these and other assays well known in the art can be used to assay for an altered phosphorylation state as compared to a control phosphorylation state.

Further provided by the invention is a method for identifying an improved effective agent that modulates a biological activity of a nuclear hormone receptor. The method includes the steps of contacting the nuclear hormone receptor with one or more agents and a eukaryotic cell sample to form a test sample under conditions suitable to form a receptor-containing complex; isolating the receptor-containing complex from the test sample; providing to the isolated receptor-containing complex conditions suitable for modification of the receptor-containing complex; assaying the isolated receptor-containing complex for an altered modification state occurring in the isolated receptor containing complex as compared to a control modification state; and assaying for direct transcriptional activity of the nuclear hormone receptor contacted with the one or more agents, where the presence of the altered modification state combined with the absence of direct transcriptional activity indicates that at least one of the one or more agents is an improved effective agent that modulates a biological activity of the nuclear hormone receptor.

A method of the invention for identifying an improved effective agent that modulates a biological activity of a hormone receptor includes the additional step of assaying for direct transcriptional activity of the hormone receptor. It is understood that the assay for direct transcriptional activity can be performed before, during, or after any of the other steps of the method. Furthermore, while the same nuclear hormone receptor such as RXR, RAR or ER, or a complex containing the receptor, is both assayed for an altered modification state and assayed for direct transcriptional activity, it is understood that different forms such as truncated derivatives, fusion proteins, variants and the like can be used in the two assays. Thus, for example, a FLAG epitope tagged truncated RXRα receptor consisting essentially of the ligand-binding domain can be used to assay an isolated receptor-containing complex for an altered modification, while a full-length RXRα receptor can be used in the assay for direct transcriptional activity.

As used herein, the term “direct transcriptional activity” means transactivation or repression occurring at a gene linked in cis to a nuclear hormone receptor response element. Direct transcriptional activity is distinguished from indirect transcriptional activity, in part, by occurring in the absence of de novo protein synthesis.

A variety of techniques are well known in the art for assaying a nuclear hormone receptor for direct transcriptional activity. In a convenient transient cotransfection assay, a first DNA construct encodes a chimeric protein containing a heterologous DNA-binding domain linked to nuclear hormone receptor ligand binding and transactivation domains, and a second DNA construct contains a cognate DNA response element linked to a reporter gene such as firefly luciferase or β-galactosidase. Upon treatment of the cells with agonist, DNA binding of the chimeric protein can result in expression and detection of the reporter gene (see, for example, Evans et al., U.S. Pat. No. 5,071,773). Additional convenient reporter systems include green fluorescent protein (Chalfie et al., Science 263:802-805 (1994)). Where one or more genes have been identified as being regulated by the nuclear hormone receptor of interest, one skilled in the art also can assay for direct transcriptional activity by directly analyzing RNA, for example, after transfection of recombinant hormone receptor (Treisman, Cell 42:889-902 (1986); Ausubel et al., supra, 2000).

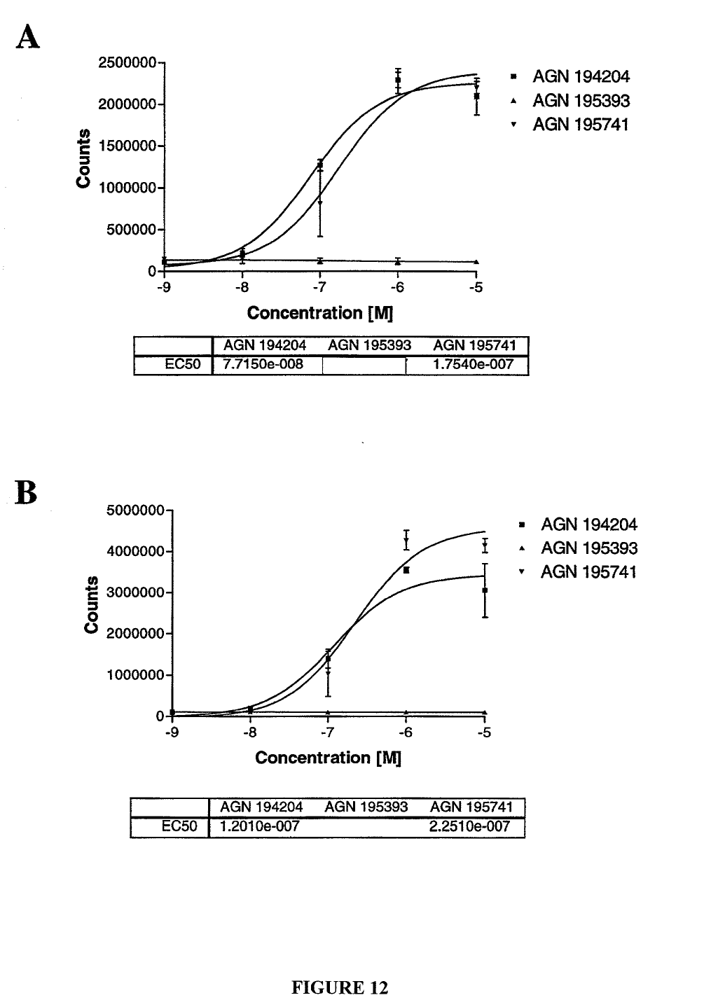

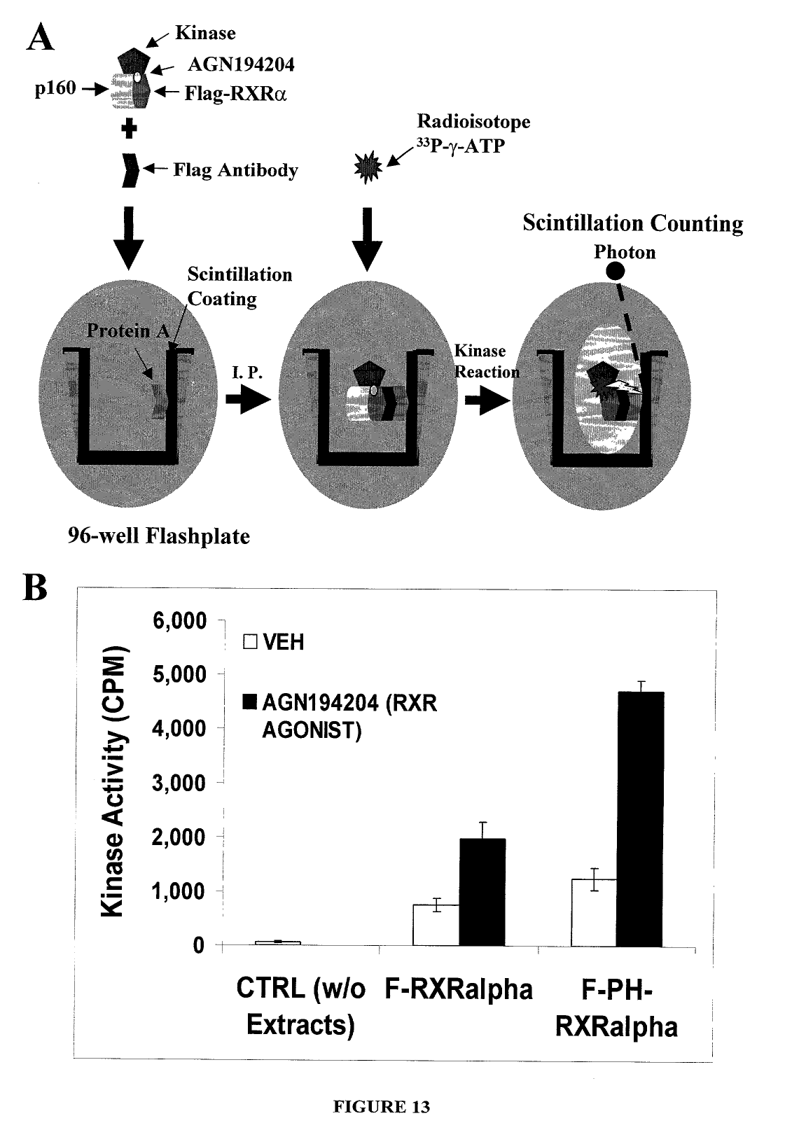

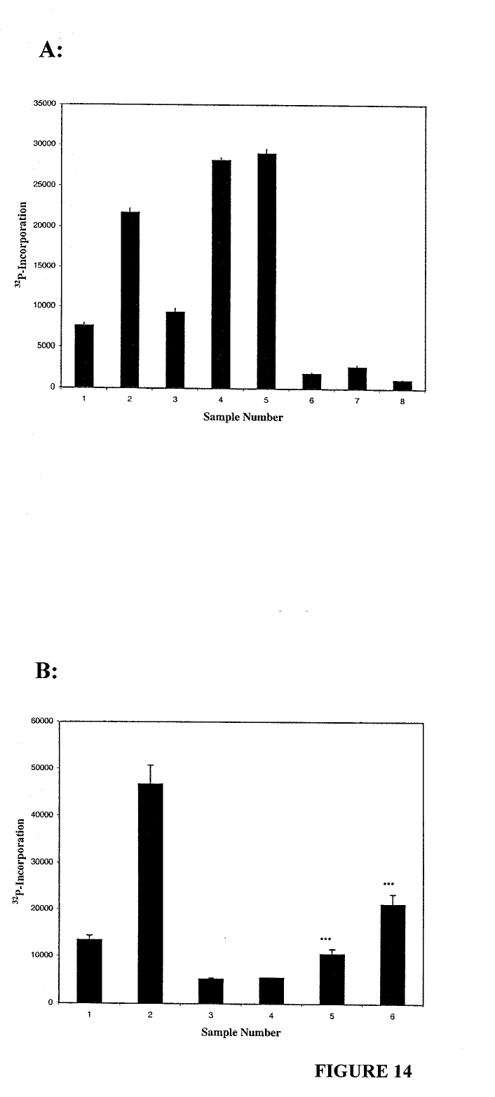

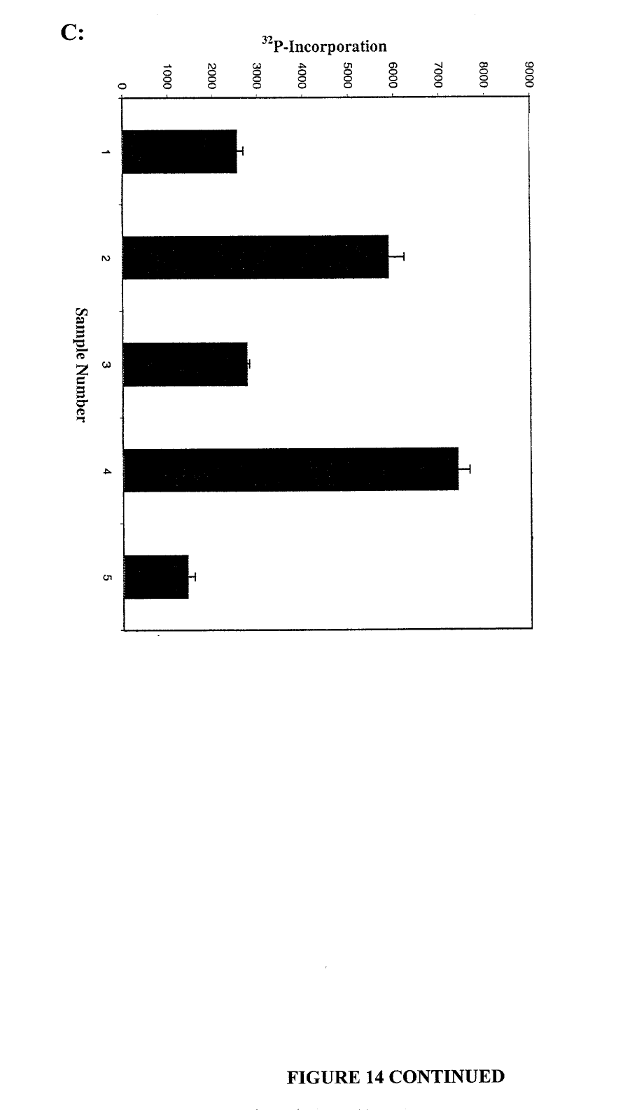

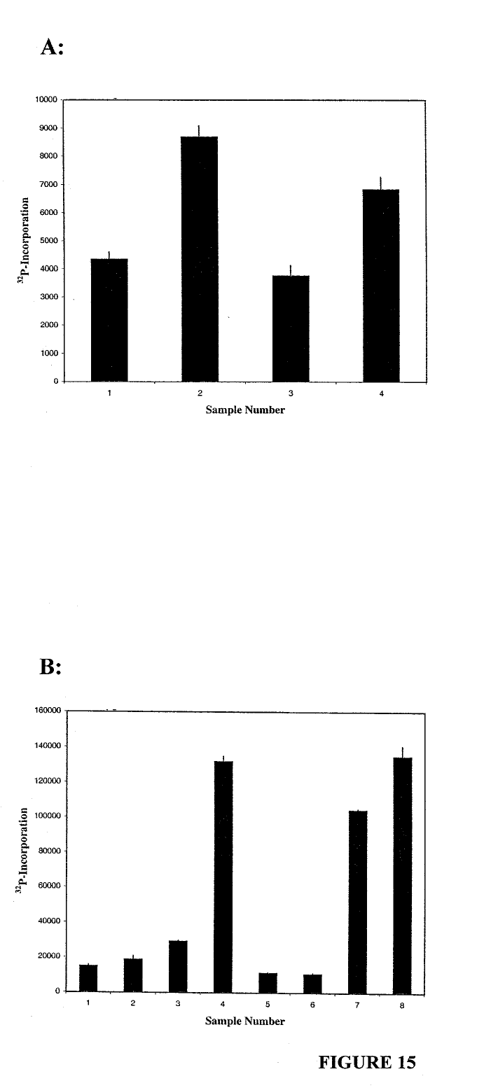

As further disclosed herein, HEK 293 cells transfected with a PH-RXRα fusion protein were treated with or without RXRα ligand AGN194204. Analysis of protein kinase A (PKA) activity associated with RXRα using an in vitro assay with synthetic peptide substrate, LRRASLG (SEQ ID NO: 59), indicated that significantly more protein kinase A activity was associated with RXRα in cells treated with AGN194024 ligand as compared to control vehicle. This activity was specifically inhibited by an inhibitor of protein kinase A, although not by an inhibitor of protein kinase C (see FIG. 14A). Similar results were obtained when PKA activity associated with RXRα was assayed with another synthetic PKA substrate, GRTGRRNSI (SEQ ID NO: 60), and with cells transfected with wild type RXRα (see FIGS. 14B and 14C). As further shown in FIGS. 15A and 15B, PKA activity associated with endogenous RXRα was ligand-dependent, as was PKA activity in cells engineered to overexpress the catalytic subunit of protein kinase A.

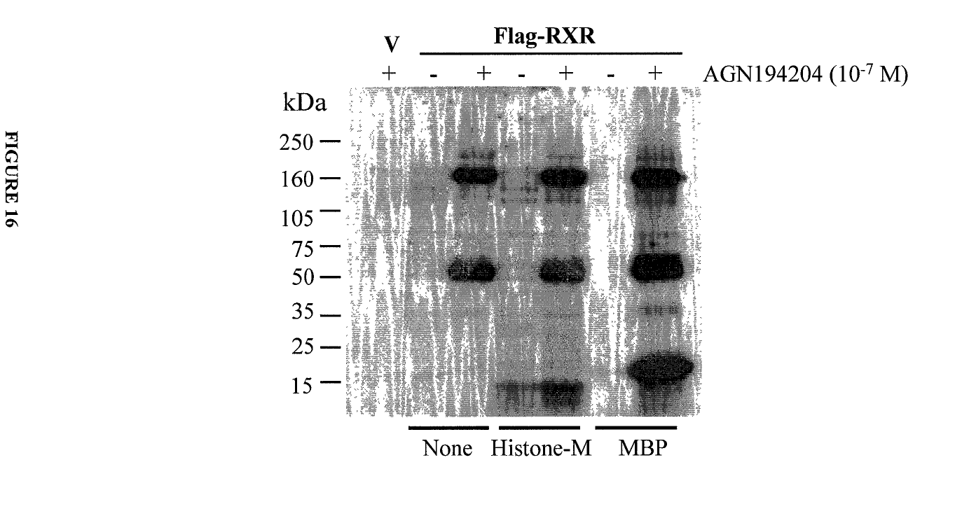

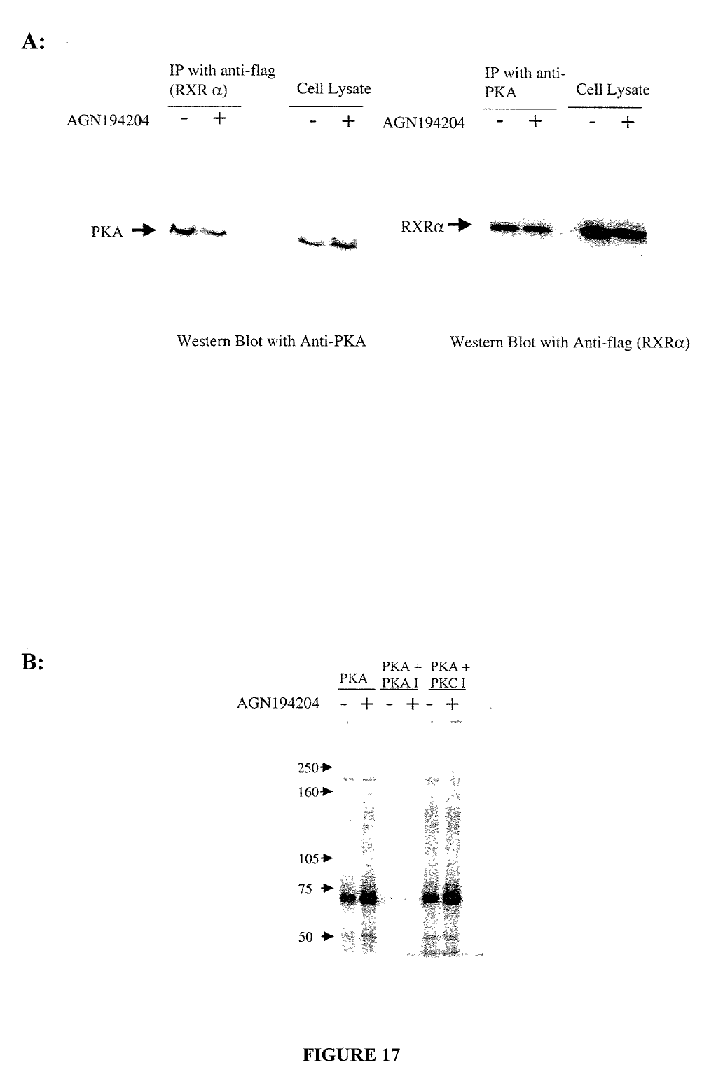

As further disclosed in FIG. 16, in vitro phosphorylation of myelin basic protein (MBP) by RXR-associated kinase was dramatically increased in Flag-RXR transfected cells treated with AGN194204. Additional results disclosed herein demonstrate a ligand-independent in vivo association between RXRα and PKA in cells overexpressing the catalytic subunit of protein kinase A. In particular, transfected cells treated with or without AGN194204 were immunoprecipitated with anti-Flag antibody and subsequently blotted with anti-protein kinase A antibody, or alternatively immunoprecipitated with anti-protein kinase A antibody and blotted with anti-Flag antibody. As shown in FIG. 17A, treatment with AGN194204 did not increase the amount of protein kinase A associated with RXRα in these cells. These results indicate that, in cells overexpressing the catalytic subunit of protein kinase A, hormone ligand can regulate the activity rather than the association of protein kinase A with hormone receptor. Additional results disclosed herein in FIG. 17B indicate that RXRα is itself phosphorylated by associated protein kinase A, and that this phosphorylation is enhanced in cells treated with RXR ligand.

Based on these discoveries, the present invention provides a method for identifying an effective agent that modulates protein kinase A activity associated with a nuclear hormone receptor. Such a method is practiced by contacting nuclear hormone receptor with one or more agents and a cell sample to form a test sample under conditions suitable to form a receptor-containing complex; isolating the receptor-containing complex from the test sample; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. In a method of the invention for identifying an effective agent that modulates protein kinase A activity associated with a nuclear hormone receptor, the altered phosphorylation state can be an increased or decreased phosphorylation state.

A substrate useful in a method of the invention can be, for example, a purified substrate and, in one embodiment, is a substrate having a Km of less than 20 μM for protein kinase A. Peptide substrates, including purified peptide substrates, are useful in the methods of the invention and, in one embodiment, a peptide substrate has at most ten residues. A peptide substrate useful in the invention can include, for example, the amino acid sequence Arg-X-Ser, Arg-Arg-X-Ser, Arg-X-X-Ser, Lys-Arg-X-X-Ser or Arg-X-Lys-Arg-X-X-Ser-X (SEQ ID NO: 113), where X is independently any amino acid. A peptide substrate useful in the methods of the invention can include, for example, the sequence Arg-Arg-X-Ser and, in particular embodiments, can contain the sequence LRRASLG (SEQ ID NO: 59) or GRTGRRNSI (SEQ ID NO: 60). In other embodiments, such a peptide substrate has a length of at most ten residues. A method of the invention also can be practiced with a purified protein substrate, for example, myelin basic protein.

The invention also provides a method for identifying an effective agent that modulates protein kinase A activity associated with a nuclear hormone receptor by contacting the nuclear hormone receptor with one or more agents and a cell sample to form a test sample under conditions suitable to form a receptor-containing complex, where the receptor is a retinoid X receptor (RXR), retinoic acid receptor (RAR) or peroxisome proliferator activated receptor (PPAR); isolating the receptor-containing complex from the test sample; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. In one embodiment, the nuclear hormone receptor is a retinoid X receptor (RXR). In another embodiment, the RXR receptor is RXRα.

A method of the invention also can be practiced with a nuclear hormone receptor that is a variant or fusion protein rather than wild type receptor. In one embodiment, the nuclear hormone receptor is a variant with an increased ratio of cytoplasmic to nuclear localization as compared to wild type nuclear hormone receptor. In another embodiment, the nuclear hormone receptor is a fusion protein that contains a heterologous membrane-anchoring domain. In a further embodiment, the nuclear hormone receptor is a fusion protein containing a pleckstrin homology domain. In yet another embodiment, the nuclear hormone receptor is a fusion protein containing a heterologous epitope tag.

A method of the invention for identifying an effective agent that modulates protein kinase A activity associated with a nuclear hormone receptor can be practiced with endogenous or exogenous receptor. In one embodiment, the invention is practiced with a cell sample containing an exogenous nucleic acid molecule encoding the nuclear hormone receptor. In another embodiment, the nuclear hormone receptor is endogenous to the cell sample. If desired, a cell sample to be used in a screening method of the invention can contain an exogenous nucleic acid molecule encoding a heterodimeric partner of the nuclear hormone receptor or an exogenous nucleic acid molecule encoding a catalytic subunit of protein kinase A. In such a cell sample, the nuclear hormone receptor can be endogenous or exogenous to the cell.

A method of the invention can be practiced with a variety of types of cell samples. In one embodiment, the cell sample contains viable eukaryotic cells, and, in another embodiment, the cell sample is a eukaryotic whole cell sample. In a method of the invention, isolation of the receptor-containing complex can be performed by a variety of means, for example, by specific binding to the receptor-containing complex. In one embodiment, isolation of the substrate is performed in the presence of the one or more agents.

Further provided by the invention is a method for identifying an effective agent that modulates protein kinase A activity associated with a nuclear hormone receptor by contacting the nuclear hormone receptor with one or more agents and a cell sample to form a test sample under conditions suitable to form a receptor-containing complex; immunoprecipitating the receptor-containing complex from the test sample to isolate the receptor-containing complex; contacting the isolated receptor-containing complex with a protein kinase A substrate under conditions suitable for phosphorylation of the substrate; and assaying the substrate for an altered phosphorylation state as compared to a control phosphorylation state, where the presence of the altered phosphorylation state indicates that at least one of the one or more agents is an effective agent that modulates protein kinase A activity associated with the nuclear hormone receptor. Immunoprecipitation can be performed, for example, using antibody immunoreactive with the nuclear hormone receptor. Where the nuclear hormone receptor is a fusion protein containing a heterologous epitope tag, the immunoprecipitation can be performed, for example, using antibody immunoreactive with the epitope tag. A variety of means can be used to assay the protein kinase A substrate for an altered phosphorylation state including, for example, detecting radiolabeled substrate.

Protein kinase A, also known as cAMP-dependent protein kinase, is the major, if not only, intracellular receptor for cAMP in mammalian cells. This kinase is composed of two genetically distinct catalytic (C) and regulatory (R) subunits. The activating ligand, cAMP, binds to the R subunit and induces conformational changes, thereby dissociating holoenzyme R₂C₂ into an R₂-(cAMP)₄ dimer and two free, catalytically active C subunits (see, for example, Cho-Chung et al., Crit. Rev. Oncology/Hematology 21:33-61 (1995)).

Protein kinase A generally is present in tissues as a mixture of type I and type II isozymes, which are distinguished by different R subunits (RI and RII) that interact with the same C subunit. Four isoforms of the R subunits, RI_{α}, RI_{β}, RII_{α} and RII_{β} and three isoforms of the C subunit (C_{α}, C_{β} and C_{γ}) have been identified. R subunits contain two tandem cAMP-binding domains within their carboxy-terminus, and exhibit high conservation in this region; the RI and RII subunits differ in their amino terminus at a proteolytically sensitive hinge region that occupies the catalytic domain of the C subunit in the holoenzyme complex. The amino terminus of the RII subunit contains the sequence Arg-Arg-X-Ser, which can be autophosphorylated, while the amino-terminus of the RI subunit contains the sequence Arg-Arg-X-Ala, a motif which cannot be autophosphorylated but which participates in high-affinity ATP binding in the holoenzyme. The RI subunit is similar in some respects to the heat-stable protein inhibitor, protein kinase inhibitor (PKI), which contains a pseudophosphorylation site; when complexed with a C subunit, the protein kinase inhibitor complex has a high-affinity ATP binding site. The RII subunit contains a region which is highly homologous to the cAMP-binding domain of bacterial catabolite gene activator protein (CAP), a DNA-binding protein, suggesting that a RII subunit also can have DNA-binding activity.

While protein kinase A initially was characterized as a cytosolic protein, PKA can undergo nuclear translocation and phosphorylate proteins in the nucleus. Furthermore, protein kinase A can have a regulated subcellular distribution and can be, for example, compartmentalized at a site of action close to a preferred substrate. In many cells and tissues, protein kinase A is predominantly found in the soluble fraction; membrane-bound protein kinase A is present in, for example, brain, erythrocytes, corpus luteum, sperm and thyroid. Membrane-bound protein kinase A can be of the type I or type II isoform, but more often is the type II isozyme.

Protein kinase A can be compartmentalized at specific sites throughout the cell through interaction of the R subunit with specific anchoring proteins designated “A kinase anchor proteins” or “AKAPs” (Scott and McCartney, Mol. Endocrinol. 8:5-11 (1994)). In brain, for example, soluble protein kinase A can be associated with microtubule structures. The high-molecular-weight microtubule associated protein, MAP₂, copurifies with type II protein kinase A and also is a substrate for the kinase. In addition, RII associates with a bovine calmodulin-binding protein known as P75 and with a related murine protein, P150. Dimerization of RII appears to be required for the interaction of the kinase with the carboxy-terminal domain of the AKAPs.

As disclosed herein, protein kinase A can be stably associated with a hormone receptor in a cell, and, furthermore, protein kinase A activity can be modulated by ligands that bind a hormone receptor that physically interacts with the kinase. A method of the invention for identifying an effective agent that modulates protein kinase A activity associated with a nuclear hormone receptor relies on a substrate for protein kinase A. As used herein, the term “protein kinase A substrate” means any molecule which contains a phosphate-accepting site and which can be detectably phosphorylated by protein kinase A in vitro. A protein kinase A substrate can be a physiological or non-physiological substrate, or a purified or unpurified substrate, and can be, for example, a synthetic peptide or peptidomimetic; a natural substrate; an exogenous substrate such as histone, myelin basic protein or casein; the RII subunit of protein kinase A itself; or a fragment of any of the above. A protein kinase A substrate generally contains a phosphate-accepting residue such as serine, threonine or an analog thereof, accompanied by a protein kinase A recognition site and generally is phosphorylated by protein kinase A with a Km of less than 4000 μM.

One skilled in the art understands that a low apparent Km for a protein substrate in a phosphorylation reaction, on the order of its concentration in vivo, is an indication that the phosphorylation is physiologically significant. However, it is understood that a protein kinase A substrate useful in the invention can be a physiological or non-physiological substrate of protein kinase A, or a fragment or analog of such a substrate. It further is understood that a protein kinase A substrate can be phosphorylated in vitro with a Km value of the same order of magnitude as the in vivo concentration of the corresponding substrate protein, or with a significantly higher or lower Km.

A protein kinase A substrate can contain one or more basic residues in proximity to a phosphate-accepting site, which generally is a serine or threonine residue or an analog thereof (see Zetterqvist et al., Peptides and Protein Phosphorylation, Chapter 7, (ed. Bruce E. Kemp), CRC Press, Inc., pp. 171-187 (1990)). A protein kinase A substrate such as a synthetic peptide substrate can include, for example, one or two arginine residues, and can include, if desired, additional arginine residues. In one embodiment, a protein kinase A substrate includes one or two arginine residues among the six amino acid residues on the amino-terminal side of a phosphate-accepting serine or threonine residue. In another embodiment, a protein kinase A substrate contains an arginine as the third amino acid preceding a phosphate-accepting serine/threonine residue; in other embodiments, this arginine is preceded or followed by a lysine residue. In a further embodiment, a protein kinase A substrate contains two arginine residues as the second and third residues preceding a phosphate-accepting serine or threonine residue. In yet another embodiment, a protein kinase A substrate includes a hydrophobic residue immediately carboxy-terminal to the phosphate-accepting serine or threonine residue.

A protein kinase A substrate useful in a screening method of the invention can contain one of a variety of protein kinase A recognition sequences that can be phosphorylated by protein kinase A in vitro. For example, the sequence Arg-Arg-X-Ser-X is phosphorylated in pyruvate kinase, glycogen synthase site 1a, phosphorylase kinase α-chain, fructose-2,6-bisphosphate/fructose-6-phosphate-2-kinase, erythrodehyroneopterin triphosphate synthetase, cytochrome P-450 LM2, tyrosine hydroxylase, and the regulatory subunit of PKA; the sequence Arg-Arg-X-Thr-X is phosphorylated in phosphoprotein phosphatase inhibitor 1 and human chorionic gonadotropin; and the sequence Arg-X-Lys-Arg-X-X-Ser-X is phosphorylated in the β-subunit of phosphorylase kinase. These proteins and fragments thereof are protein kinase A substrates useful in the methods of the invention.

Additional protein kinase A substrates useful in the invention include but are not limited to the following: acetyl-CoA carboxylase; acetyl-CoA carboxylase kinase; arylsulfatase B; ATP citrate lyase; cholesterol esterase; cyclic nucleotide phosphodiesterase; erythrodihydroneopterin triphosphate synthetase; F 1-ATPase precursor; fructose-1,6-bisphosphatase; fructose-2,6-bisphosphatase/fructose-6-phosphate-2-kinase; muscle fructose-6-phosphate-1-kinase; liver fructose-6-phosphate-1-kinase; muscle glycogen synthase; liver glycogen synthase; guanylate cyclase; hormone-sensitive lipase/diglyceride lipase; myosin light-chain kinase; Na⁺, K⁺-ATPase, α-subunit; phenylalanine hydroxylase; phospholipid methyltransferase; phosphorylase kinase; pyruvate kinase, liver; erythrocyte pyruvate kinase; RNA polymerase; PKA Catalytic subunit; PKA Regulatory subunit (R^{II}); threonyl-tRNA synthetase; or tyrosine hydroxylase, and fragments of any of the above proteins. Protein kinase A substrates useful in the invention further include, for example, actin; atrial natriuretic peptides; calciductin; dihydropyridine-sensitive calcium channel; choriogonadotropin; collagen, αI; cytochrome P-450 LM2; fibrinogen; filamin; G-substrate; glicentin; glucocorticoid receptor; histone; HMG 14; keratin proteins; lens α-crystallin; lipomodulin; MAP-2; myelin basic protein; phosphatase inhibitor 1; phospholamban; prolactin; ribosomal protein S6; sodium channel α-subunit; or cardiac or skeletal muscle troponin I, and fragments of any of the above proteins (Zetterqvist et al., supra, 1990). It is understood that these and other protein kinase A substrates known in the art can be useful in the methods of the invention, and further that routine methods can be used to identify additional protein substrates, fragments thereof, or synthetic substrates.

The protein kinase A recognition sequences in a variety of protein substrates of protein kinase A are shown in Table 2 (see, also, Zetterqvist et al., supra, (1990); and Pearson and Kemp, Methods in Enzymology, 200:62-81 (1991)). Additional protein kinase A substrate sequences are shown in Table 3. It is understood that recombinant or proteolytic methods can be used, for example, to prepare protein kinase A substrates containing one of the sequences shown in Table 2 or Table 3. If desired, synthetic peptides containing these sequences or other protein kinase A recognition sites can be prepared for use as a protein kinase A substrate in a method of the invention. Additional protein kinase A substrates include proteins engineered to contain one or more protein kinase A recognition sites (see Pestka et al., Protein Expr. Purif. 17:203-214 (1999)). It is understood that a variety of proteins can be engineered to contain a protein kinase A recognition site, including proteins that contain a naturally occurring protein kinase A recognition site as well as proteins that are not natural substrates for protein kinase A.

TABLE 2
SELECTED PROTEIN KINASE A PHOSPHORYLATION SITES

| Phosphorylation | | | |
|---|---|---|---|
| site sequence/ | | Protein | |
| SEQ ID NO. | Protein | Reference | |
| Y₇ L R R A S L/V A Q L T | Pyruvate | Zetterqvist | |
| SEQ ID NO.: 61 | kinase^{a} | et al., | |
| | | “Peptides | |
| | | and Protein | |
| | | Phosphoryla | |
| | | tion,” | |
| | | Uniscience | |
| | | CRC Press, | |
| | | Boca Raton, | |
| | | FA (1990) | |
| F₁ R R L S I S T | Phosphoryl- | Zetterqvist | |
| SEQ ID NO.: 62 | ase kinase | et al., | |
| | α chain | supra, 1990 | |
| Q₆₉₂ W P R R A S C T S | Glycogen | Zetterqvist | |
| SEQ ID NO.: 63 | synthase | et al., | |
| | site 1a^{b} | supra, | |
| | | 1990; | |
| | | Browner et | |
| | | al., Proc. | |
| | | Natl. Acad. | |
| | | Sci. U.S.A. | |
| | | 86:1443 | |
| | | (1989) | |
| G₇₀₆ S K R S N S V D T | Glyogen | Zetterqvist | |
| SEQ ID NO.: 64 | synthase | et al., | |
| | site 1b^{b} | supra, | |
| | | 1990; | |
| | | Browner et | |
| | | al., supra, | |
| | | (1989) | |
| R₂₁ T K R S G S V/I Y E | Phosphoryl- | Zetterqvist | |
| SEQ ID NO.: 65 | ase kinase | et al., | |
| | β chain^{a} | supra, 1990 | |
| A₂₉ G A R R K A S G P P | Histone | Zetterqvist | |
| SEQ ID NO.: 66 | H1^{a,c} | et al., | |
| | | supra, 1990 | |
| K₁₃ A K T R S S R A | Histone | Zetterqvist | |
| SEQ ID NO.: 67 | H2A^{a,c} | et al., | |
| | | supra, 1990 | |
| G₂₆ K K R K R S R K E S Y | Histone | Zetterqvist | |
| S | H2B^{a,c} | et al., | |
| SEQ ID NO.: 68 | | supra, 1990 | |
| E R R K S K S G A G | cAMP | Hemmings et | |
| SEQ ID NO.:69 | regulated | al., | |
| | phospho- | J. Biol. | |
| | protein | Chem. | |
| | Mᵣ=21,000 | 264:726 | |
| | (ARPP-21) | (1989) | |
| Y₃ L R R R L S D S N F | Synapsin I | Hall et | |
| SEQ ID NO.: 70 | site 1 | al., J. | |
| | | Biol. Chem. | |
| | | 265:6944 | |
| | | (1990) | |
| N₁₉ Y R G Y S L G N Y V | Reduced | Bylund and | |
| SEQ ID NO.: 71 | carboxy- | Krebs, J. | |
| | methylated | Biol. Chem. | |
| | maleylated | 250: 6355 | |
| | (RCMM)- | (1975) | |
| | lysozyme | | |
| R₂₇ A S F G S R G S G S | Desmin | Kitamura et | |
| SEQ ID NO.: 72 | | al., J. | |
| | | Biol. Chem. | |
| | | 264:5674 | |
| | | (1989) | |
| S₄₇ R T S A V P T | Desmin | Kitamura et | |
| SEQ ID NO.: 73 | | al., supra, | |
| | | 1989 | |
| P₁ L S R T L S V S S | Glycogen | Zetterqvist | |
| SEQ ID NO.: 74 | synthase | et al., | |
| | site 2 | supra, 1990 | |
| A₁₆ V R R S D R A | Troponin I | Kemp, J. | |
| SEQ ID NO.: 75 | (Cardiac) | Biol. Chem. | |
| | | 254:2638 | |
| | | (1979) | |
| N₅₆₀ R R S V S E A A L | Hormone- | Hardie et | |
| SEQ ID NO.: 76 | sensitive | al., Trends | |
| | lipase | Biochem; | |
| | | Sci. 14:20 | |
| | | (1989) | |
| N₇₄ R S S M S G L H L | Acetyl-CoA | Hardie et | |
| SEQ ID NO.: 77 | carboxylase | al., supra, | |
| | | 1989 | |
| S₁₂ Q R R R S L E P P D | pp60^{c-src} | Patschinsky | |
| SEQ ID NO.: 78 | | et al., J. | |
| | | Viral. | |
| | | 591:73 | |
| | | (1986) | |
| K₂₃ R K R K S S Q C L V K | c-erbA | Goldberg et | |
| SEQ ID NO.: 79 | | | al., EMBO |
| | | J. 7:2425 | |
| | | (1988) | |
| K₁₁ H K R K S S Q C L V K | v-erbA | Goldberg et | |
| SEQ ID NO.: 80 | | al., supra, | |
| | | 1988 | |
| R₄₅ N T D G S T D Y G I | RCMM- | Bylund and | |
| SEQ ID NO.: 81 | lysozyme | Krebs, | |
| | | supra, 1975 | |
| R₂₁₂ R K G T D V | Lipocortin | Varticovski | |
| SEQ ID NO.: 82 | I (p35, | et al., | |
| | calpactin | Biochemistry | |
| | II) | 27:3682 | |
| | | (1988) | |
| I₂₉ R R R R P T P A T | Phosphatase | Zetterqvist | |
| SEQ ID NO.: 83 | inhibitor- | et al., | |
| | 1^{a} | supra, 1990 | |
| K P R R K D T P A L | G substrate | Zetterqvist | |
| SEQ ID NO.: 84 | | et al., | |
| | | supra, 1990 | |
| K₁₈₈ R V K G R T W T L C G T | Autophos- | Zetterqvist | |
| SEQ ID NO.: 85 | phorylation | et al., | |
| | of | Supra | |
| | catalytic | (1990); | |
| | subunit | Shoji et | |
| | | al., Proc. | |
| | | Natl. Acad. | |
| | | Sci. U.S.A. | |
| | | 78:848 | |
| | | (1981) | |
| R₈₉ F D R R V S V C A | Autophos- | Zetterqvist | |
| SEQ ID NO.: 86 | phorylation | et al., | |
| | of | supra, 1990 | |
| | regulatory | | |
| | subunit R11 | | |

Yeast

| | | |
|---|---|---|
| K₂₁₈ R K Y L K K L T R R A | ADR1 | Cherry et |
| S F S A | | al., Cell |
| SEQ ID NO.: 87 | | 56:409 |
| | | (1989) |
| P₇ R R D S T E G F | Fructose-1, | Rittenhouse |
| SEQ ID NO.: 88 | 6- | et al., J. |
| | bisphospha- | Biol. Chem. |
| | tase | 262:10114 |
| | | (1987) |
| Q₁₄₁ R R T S V S G E | Autophos- | Kuret et |
| SEQ ID NO.: 89 | phorylation | al., J. |
| | of | Biol. Chem. |
| | regulatory | 263:9149 |
| | subunit | (1988) |

^{a}Residue number obtained from SWISSPROT
^{b}Residue number based on human muscle sequence
^{c}Residue number from bovine sequence

TABLE 3
SELECTED PROTEIN KINASE A SYNTHETIC SUBSTRATES

| Amino Acid Sequence/ | Kₘ | |
|---|---|---|
| SEQ ID NO. | (μM) | Reference |
| G R G L S L S R | 240 | Daile et al., |
| SEQ ID NO.: 90 | | Nature 257:416 |
| | | (1975) |
| L R R A S V A | <10 | Zetterqvist et al., |
| SEQ ID NO.: 91 | | Biochem. Biophys. |
| | | Res. Commun. 70:696 |
| | | (1976) |
| R R A S V A | 20 | Zetterqvist et al., |
| SEQ ID NO.: 92 | | supra, (1976) |
| R R A S V | 24^{b} | Hider et al., Bio- |
| SEQ ID NO.: 93 | | chem. J. 229:435 |
| | | (1935) |
| L R R A S L G | 16 | Kemp et al., J. |
| SEQ ID NO.: 94 | | Biool. Chem. 252: |
| | | 4888 (1977) |
| R R A S L G | 26 | Kemp et al., supra, |
| SEQ ID NO.: 95 | | 1977 |
| L R R A S L | 57 | Kemp et al., supra, |
| SEQ ID NO.: 96 | | 1977 |
| V L Q R R R G S S I P Q | 4 | Glass et al., J. |
| SEQ ID NO.: 97 | | Biol. Chem. 261: |
| | | 2987 (1986) |
| A R T K R S G S V | 10 | Zetterqvist and |
| SEQ ID NO.: 98 | | Ragnarsson, FEBS |
| | | Lett. 139:287 |
| | | (1982) |
| R T K R S C S V | 21 | Zetterqvist and |
| SEQ ID NO.: 99 | | Ragnarsson, supra, |
| | | (1982); Ragnarsson |
| | | et al., Peptides |
| | | 1978 p. 339 (1979) |
| L R K A S L G | 260 | Kemp et al., supra, |
| SEQ ID NO.: 100 | | 1977 |
| L H R A S L G | 415 | Kemp et al., supra, |
| SEQ ID NO.: 101 | | 1977 |
| L X^{a} R A S L G | 350 | Kemp et al., supra, |
| SEQ ID NO.: 102 | | 1977 |
| V L Q R R R G S S I P Q | 4 | Glass et al., |
| SEQ ID NO.: 103 | | supra, 1986 |
| V L Q A R R G S S I P Q | 11 | Glass et al., |
| SEQ ID NO.: 104 | | supra, 1986 |
| R T K R S G S V | 21 | Zetterqvist and |
| SEQ ID NO.: 105 | | Ragnarsson, supra, |
| | | (1982); Ragnarsson |
| | | et al., supra, |
| | | (1979) |
| R T G R S G S V | 140 | Zetterqvist and |
| SEQ ID NO.: 106 | | Ragnarsson, supra, |
| | | (1982) |
| R R R R P T P A | 296- | Chessa et al., Eur. |
| SEQ ID NO.: 107 | 1,300 | J. Biochem. 135:609 |
| | | (1983) |
| V L Q R R R G T S I P Q | 39 | Glass et al., |
| SEQ ID NO.: 108 | | supra, 1986 |
| V L Q A R R G T S I P Q | 139 | Glass et al., |
| SEQ ID NO.: 109 | | supra, 1986 |
| V L Q R R R P T S I P Q | 118 | Glass et al., |
| SEQ ID NO.: 110 | | supra, 1986 |
| R R A S F | 13 | ------------------- |
| SEQ ID NO.: 111 | | -- |
| R R A S I | 7 | ------------------- |
| SEQ ID NO.: 112 | | -- |

X^{a} = homoarginine

The phosphate-accepting site that has the potential to be phosphorylated by protein kinase A in a protein kinase A substrate is denoted a “phosphorylatable” site. A protein kinase A substrate useful in the invention can have a single phosphate-accepting site, or two or more, three or more, five or more, ten or more, or twenty or more phosphate-accepting sites. Furthermore, a protein kinase A substrate useful in the invention can contain multiple identical or distinct recognition sequences accompanying the phosphate-accepting site. A protein kinase A substrate useful in the invention can contain, for example, a repeated phosphate-accepting recognition sequence such as (Arg-X₁-Ser)ₙ (SEQ ID NO: 137), where n is 2 to 20 and each X₁ is independently any amino acid; (Arg-Arg-X₁-Ser-X₂)ₙ (SEQ ID NO: 138), where n is 2 to 20 and each X₁ and each X₂ each is independently any amino acid; (Arg-X₁-X₂-Ser)ₙ (SEQ ID NO: 139), where n is 2 to 20 and each X₁ and each X₂ each is independently any amino acid; (Lys-Arg-X₁-X₂-Ser)ₙ (SEQ ID NO: 140), where n is 2 to 20 and each X₁ and each X₂ each is independently any amino acid; or (Arg-X₁-Lys-Arg-X₂-X₃-Ser-X₄)ₙ (SEQ ID NO: 141), where n is 2 to 20 and each X₁, each X₂, each X₃, and each X₄ each is independently any amino acid. In specific embodiments, any of the above repeats are identically repeated 2 to 20 times; that is, each X₁ is the same residue throughout the substrate; each X₂, if present, is the same residue throughout the substrate; each X₃, if present, is the same residue throughout the substrate; and each X₄, if present, is the same residue throughout the substrate. Thus, a protein kinase A substrate useful in the invention can contain multiple repetitions of a phosphorylatable protein kinase A recognition sequence, which can be any of the motifs disclosed herein, for example, as shown herein in Tables 2 and 3. Thus, any of the sequences shown herein in Table 2 or 3 can be repeated, for example, two or more, three or more, four or more, five or more, ten or more, or twenty or more times. If desired, a protein kinase A substrate can contain one or more additional sequences, including repeated or non-repeated sequences, intervening between the repeated phosphorylatable protein kinase A recognition sequences. A recombinant protein that is a natural substrate for protein kinase A and which has been engineered to contain one or more phosphorylatable sites is another example of a protein kinase A substrate that contains multiple phosphate-accepting sites. Methods for preparing such recombinant protein substrates are well known in the art, as described, for example, in Pestka et al., supra, 1999.

A protein kinase A substrate useful in the invention can have a variety of lengths. A protein kinase A substrate, such as a peptide substrate, can have a length, for example, of three or more, four or more, five or more, six or more, seven or more, eight or more, nine or more, ten or more, 11 or more, 12 or more, 13 or more, 14 or more, 15 or more, 16 or more, 17 or more, 18 or more, 19 or more, 20 or more, 25 or more, 30 or more, 40 or more, 60 or more, or 80 or more residues. A protein kinase A substrate useful in the invention also can have a length of at most three, at most four, at most five, at most six, at most seven, at most eight, at most nine, at most ten, at most 11, at most 12, at most 13, at most 14, at most 15, at most 16, at most 17, at most 18, at most 19, at most 20, at most 25, at most 30, at most 35, at most 40, at most 60, at most 80, or at most 100 residues. In particular embodiments, a protein kinase A substrate has a length of three to thirty residues, four to thirty residues, five to thirty residues; five to twenty residues; five to fifteen residues; five to twelve residues; five to ten residues; six to thirty residues; six to twenty residues; six to fifteen residues; six to twelve residues; six to ten residues; seven to thirty residues; seven to twenty residues; seven to fifteen residues; seven to twelve residues; seven to ten residues; eight to thirty residues; eight to twenty residues; eight to fifteen residues; eight to twelve residues; or eight to ten residues.

A protein kinase A substrate useful in the invention can have a relatively low apparent Km or can have a higher apparent Km. For use in the methods of the invention, a protein kinase A substrate can have, for example, a Km of at most 4000 μM, a Km of at most 50 μM or, for example, a Km in the range of 1 to 10 μM. In specific embodiments, a protein kinase A substrate has a Km of at most 5 μM, at most 10 μM, at most 15 μM, at most 20 μM, at most 30 μM, at most 40 μM, at most 50 μM, at most 100 μM, at most 250 μM, at most 500 μM or at most 1000 μM. In other embodiments, a protein kinase A substrate has a Km of more than 5 μM, more than 10 μM, more than 15 μM, more than 20 μM, more than 30 μM, more than 40 μM, more than 50 μM, more than 100 μM, more than 250 μM, more than 500 μM or more than 1000 μM. In still further embodiments, a protein kinase A substrate has a Km of 1 to 500 μM, 1 to 250 μM, 1 to 100 μM, 1 to 50 μM, 1 to 20 μM, or 1 to 10 μM.

It is recognized that a protein kinase A substrate useful in the invention need not be specific for protein kinase A but additionally can be phosphorylated by one or more other kinases at the same or a different phosphate-accepting site. In one embodiment, a protein kinase A substrate useful in the invention is a substrate that is selectively phosphorylated by protein kinase A in preference to most other protein kinases. Such a selective protein kinase A substrate is phosphorylated by protein kinase A with a Km of at most 50 μM but is phosphorylated by other kinases with a Km which is at least an order of magnitude greater than the Km of the substrate for protein kinase A. In another embodiment, a protein kinase A substrate is non-selectively phosphorylated. A protein kinase A substrate also can be a substrate that is specifically phosphorylated by protein kinase A and is not significantly phosphorylated by other kinases. Kemptide (LRRASLG; SEQ ID NO: 59), for example, is a selective protein kinase A substrate useful in the invention. In addition, peptide GRTGRRNSI (SEQ ID NO: 60) is a specific protein kinase A substrate useful in the invention. SEQ ID NO: 60 has a Km =0.11 μM, which is approximately 40-fold lower than the Km of kemptide (SEQ ID NO: 59), as well as a Vmax about 2-fold higher than that of kemptide. It is understood that the selectivity and specificity of substrates for protein kinase A and other protein kinases can be modulated, if desired, by amino acid substitutions or other modifications of a base recognition sequence (Kemp and Pearson, Methods in Enzymology 200:121-134 (1991)).

Km=0.11 μM; approximately 40-fold lower Km and 2-fold higher Vmax than kempeptide. Kempeptide is considered to be fairly specific substrate of PKA.

A protein kinase A substrate also can be a peptidomimetic. As used herein, the term “peptidomimetic” is used broadly to mean a peptide-like molecule that has the activity of the peptide substrate upon which it is structurally based. Such peptidomimetics include chemically modified peptides, peptide-like molecules containing non-naturally occurring amino acids, and peptoids that contain a phosphate-accepting site and that can be detectably phosphorylated by protein kinase A in vitro (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861). It is understood that such a peptidomimetic can have substantially the same Km and selectivity as the peptide substrate upon which it is based, or can have an increased or decreased Km or selectivity as compared to the corresponding peptide substrate.

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; α,α-dialkylglycine or α-aminocycloalkane carboxylic acid; an N^{α}-C^{α} cylized amino acid; an N^{α}-methylated amino acid; a β- or γ-amino cycloalkane carboxylic acid; an α,β-unsaturated amino acid; a β,β-dimethyl or β-methyl amino acid; a β-substituted-2,3-methano amino acid; an N-C^{δ} or C^{α}-C^{δ} cyclized amino acid; a substituted proline or another amino acid mimetic. A peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylenesulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics can be useful as protein kinase A substrates in the methods of the invention.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a synthetic protein kinase A peptide substrate, as well as potential geometrical and chemical complementarity to protein kinase A. Where no crystal structure of a peptide substrate is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify peptidomimetic protein kinase A substrates useful in the invention.

A protein kinase A substrate useful in the invention can be in purified or unpurified form. As used herein, the term “purified substrate” means a protein kinase A substrate in a form that is substantially isolated from other peptides and proteins as well as other components with which the substrate is normally associated in a cell. In one embodiment, a purified substrate is at least 95% pure by weight.

A protein kinase A substrate useful in the invention also can be provided in partially purified form, for example, as part of a fractionated cell extract or as the product of a single purification step. Such a protein kinase A substrate can be, for example, part of a bacterial or mammalian cell extract prepared from a cell that expresses an exogenous nucleic acid molecule encoding a protein kinase A substrate. Methods for expressing a recombinant protein in a bacterial or eukaryotic cell are well known in the art and described hereinabove. Where the protein kinase A substrate is provided in unpurified or partially purified form, it is understood that, in general, little significant protein kinase A activity is provided with the substrate. A substrate can be assayed for co-purifying protein kinase A activity by routine methods described herein.

Methods of preparing protein, peptide and peptidomimetic substrates of protein kinase A are well known in the art. Merrifield solid-phase synthesis with tBoc or fMoc chemistries, for example, is well known in the art for preparation of synthetic peptides (Kent, Annu. Rev. Biochem. 57:957 (1988)). Automated peptide synthesizers such as the Applied Biosystems Model 430 (Foster City, Calif.) can be used for preparation of a synthetic peptide protein kinase A substrate. Synthetic peptides can be purified by standard methods, for example, using a combination of ion-exchange and and reversed-phase chromatography or using a two-step reversed phase chromatography (see, for example, Kemp and Pearson, supra, (1991)).

If desired, a protein kinase A substrate can be expressed as a fusion protein or peptide, for example, with a tag suitable for purification or partial purification. A protein kinase A substrate can be expressed, for example, as a GST fusion protein, HA fusion, or another fusion as described herein above. Those skilled in the art understand that, where a tagged receptor fusion protein is used in a method of the invention, any tag used on the substrate must be distinct from the tag fused to the hormone receptor.

As disclosed herein, a fusion protein containing the RXRα receptor and a heterologous pleckstrin homology (PH) domain was associated with hormone ligand-regulated protein kinase A activity. The ligand-dependence of the PKA activity was enhanced in cells containing the PH-RXRα fusion protein as compared to RXRα receptor that lacked a fused pleckstrin homology domain. Thus, in one embodiment, a nuclear hormone receptor to be used in a screening method of the invention is a fusion protein that contains a pleckstrin homology domain.

As used herein, the term “pleckstrin homology domain” means a domain with a characteristic β-sandwich structure and limited primary sequence homology to a repeated region in the protein pleckstrin. The “pleckstrin homology domain” was originally identified in pleckstrin, a major protein kinase C (PKC) substrate in activated blood platelets; this protein contains two pleckstrin homology domains. Pleckstrin homology domains generally are small domains of about 120 amino acids. Pleckstrin homology domains are present in, for example, serine/threonine kinases such as Akt/Rac/PKB; GTPase activating proteins and guanine nucleotide exchange factors; cytoskeletal proteins such as spectrin; signalling adapter molecules such as Grb7; and proteins involved in cellular membrane transport, for example, hSec7 and dynamin. Over 100 pleckstrin homology domains have been identified. See, for example, Gibson et al., Trends. Biochem. Sci. 19:349-353 (1994); Musacchio et al., Trends Biochem. Sci. 18:343-348 (1993); and Shaw et al., Bioessays 18:35-46 (1996)).

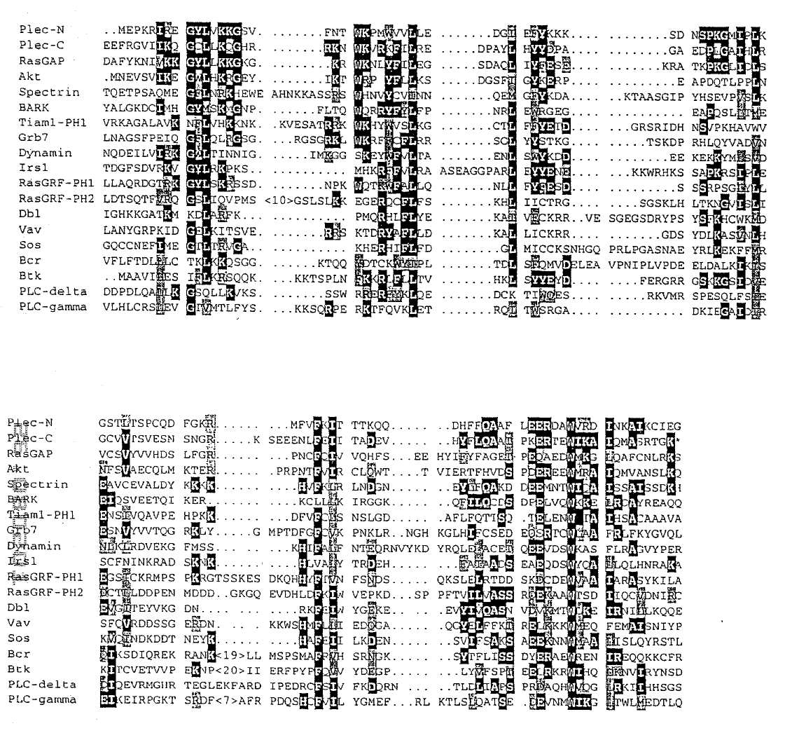

The sequence alignment of 19 selected pleckstrin homology domains is shown in FIG. 18 (Lemmon and Ferguson, Current Topics Micro. Immunol. 228:39-74 (1998)). The overall percentage of amino acid identity varies from less than 10% to about 30% for pleckstrin homology domains from unrelated proteins (see FIG. 18). Conserved amino acids in pleckstrin homology domains include a tryptophan close to the carboxy-terminus, found in nearly all PH domains. In the carboxy-terminal region of about 15 amino acids containing the conserved tryptophan, there generally are at least one and often two or more negatively changed amino acids, especially glutamic acid. The fourth residue carboxy-terminal to the tryptophan typically is a large hydrophobic Phe, Leu, Iso, or Val residue. The region of about ten residues surrounding the tryptophan generally does not contain proline, a helix-breaking residue. The amino-terminal region of about 85 amino acids generally is rich in large hydrophobic, turn-promoting and positively charged residues; and a glycine typically precedes the first β-strand. In addition, a pleckstrin homology domain generally contains six sequence blocks with a conserved pattern of hydrophobic and hydrophilic residues. At the level of secondary structure, a pleckstrin homology domain generally consists of 7 or 8 β-strands and a single carboxy-terminal α-helix (Lemmon and Ferguson, supra, 1998; Shaw, supra, 1996).

A variety of pleckstrin homology domains are known in the art, including but not limited to pleckstrin homology domains from the following proteins: human pleckstrin (residues 1 to 105; Tyers et al., Nature 333:470-473 (1988)); human pleckstrin (residues 239 to 350; Tyers et al., supra, (1988)); human Ras GTPase activating protein (residues 292 to 404; Trahey et al., Science 242:1697-1700 (1988)); human serine/threonine kinase AKT2 (residues 1 to 118; Cheng et al., Proc. Natl. Acad. Sci. USA 89:9267-9271 (1992)); human β-spectrin (residues 2192 to 2311 (Hu et al., J. Biol. Chem. 267:18715-18722 (1992)); human β-adrenergic receptor kinase (residues 553 to 656; Chuang et al., J. Biol. Chem. 267:6886-6892 (1992)); mouse GDP-GTP exchanger Tiam-1 (residues 440 to 546; Habets et al., Cell 77:537-549 (1994)); mouse growth factor receptor bound protein 7 (residues 224 to 345; Margolis et al., Proc. Natl. Acad. Sci. USA 89:8894-8898 (1992)); human GTPase dynamin 1 (residues 515 to 629; van der Bliek et al., J. Cell Biol. 122:553-563 (1993)); rat insulin receptor substrate 1 (residues 7 to 119; Sun et al., Nature 352:73-77 (1991)); rat Ras guanine nucleotide release factor (residues 17 to 134; Shou et al., Nature 358:351-354 (1992)); rat Ras guanine nucleotide release factor (residues 451 to 584; Shou et al., supra, (1992)); human product of the dbl proto-oncogene (residues 704 to 813; Ron et al., EMBO J. 7:2465-2473 (1988)); mouse product of the vav proto-oncogene (residues 397 to 508; Adams et al., Oncogene 7:611-618 (1992)); human son of sevenless protein (residues 438 to 548; Chardin et al., Science 260:1338-1343 (1993)); human break point cluster region gene product (residues 728 to 870; Lifshitz et al., Oncogene 2:113-117 (1988)); human Bruton's tyrosine kinase (residues 1 to 137; Vetrie et al., Nature 361:226-233 (1993)); rat phospholipase Cδ₁ (residues 15 to 134; Suh et al., Cell 54:161-169 (1988)); human phospholipase Cγ₁ (residues 22 to 146; Burgess et al., Mol. Cell. Biol. 10:4770-4777 (1990)).

It is understood that a pleckstrin homology domain functions independently of its location in a protein and can be positioned at the amino-terminus, carboxy-terminus or internally. A pleckstrin homology domain can function in recruitment to the cell membrane, for example, the plasma membrane through interaction with specific membrane components. When expressed as a fusion with a nuclear hormone receptor, a pleckstrin homology domain can function to promote an increased ratio of cytoplasmic to nuclear localization as compared to wild type hormone receptor.

It is understood that synthetic, modified and newly identified pleckstrin homology domains can be fused to a nuclear hormone receptor for use in a screening method of the invention in addition to the domains set forth above. It further is understood that a pleckstrin homology domain useful in the invention can correspond to a naturally occurring pleckstrin homology domain, or can contain one or more amino acid additions, substitutions or deletions relative to a naturally occurring domain, provided that the pleckstrin homology domain retains substantially the ability to promote an increased ratio of cytoplasmic to nuclear localization of a fused nuclear hormone receptor.

All journal article, reference, and patent citations provided above, in parentheses or otherwise, whether previously stated or not, are incorporated herein by reference.

The following examples are intended to illustrate but not limit the present invention.

### EXAMPLE I

### Rapid Activation of Map Kinases by RXR-specific Ligand

This example demonstrates that MAP kinases can be rapidly phosphorylated in response to an RXR-specific agonist.

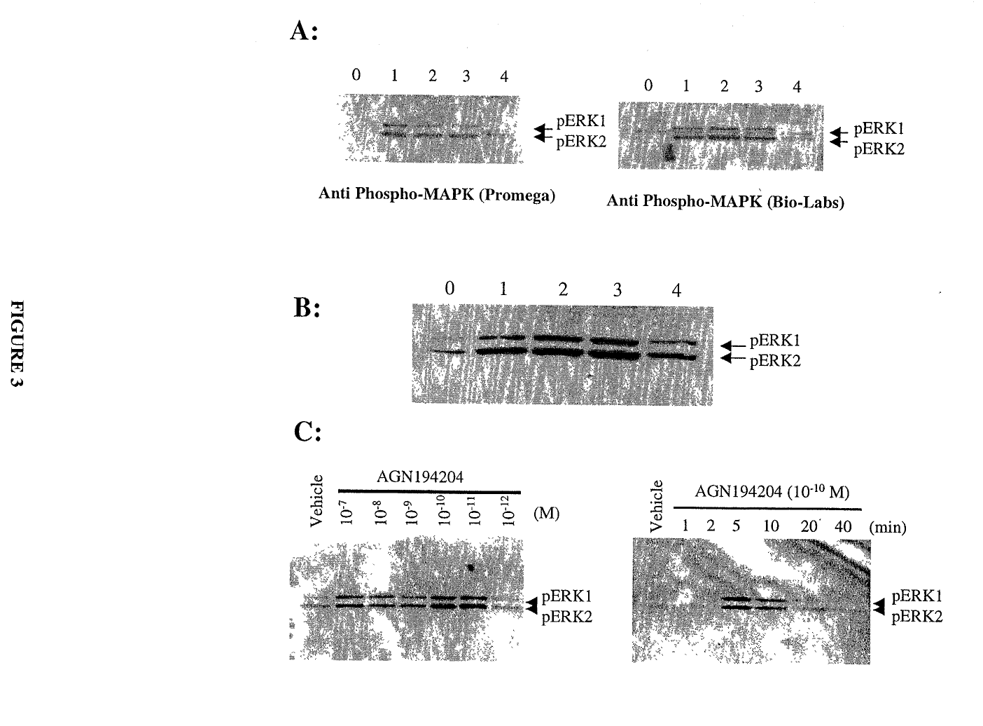

MAP kinase (MAPK or ERK1/2) is activated by phosphorylation on Thr and Tyr residues of a TEY motif by the dual-specific MAP kinase kinase (MEK). In order to determine whether RXR-specific ligand can activate MAP kinase, antibodies specific for phosphorylated forms of MAP kinase (ERK1/2) were used to probe for phosphorylated MAP kinase in cells treated with vehicle or with the RXR-specific agonist, AGN194204. As shown in FIGS. 2A and 2B, exposure of 3T3-L1 fibroblasts or differentiated adipocytes to the RXR-specific agonist increased phosphorylation of MAP kinase. The increased phosphorylation was observed within 5 minutes of treatment with AGN194204 and had a duration of about 20 minutes. As further shown in FIG. 3C, the response was dose-dependent (0.01-100 nM), with maximal stimulation at 0.1 nM.

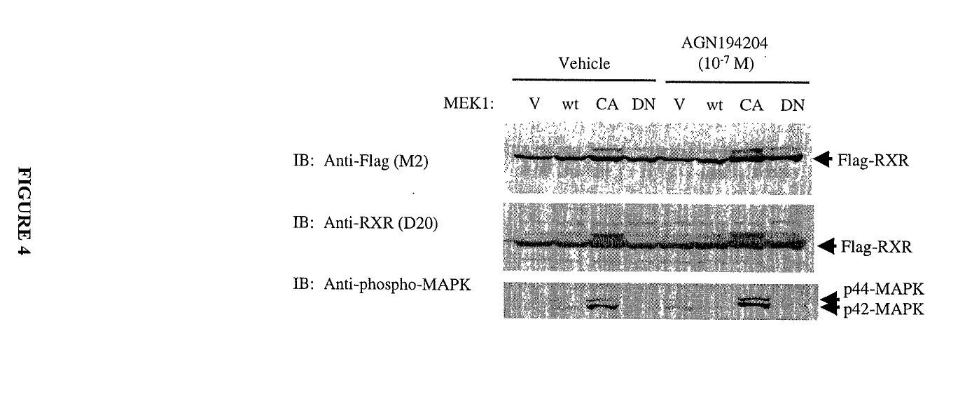

MEK1 can be activated by phosphorylation on two serine residues, Serine 217 and Serine 221, which are located within the catalytic domain of the kinase. Dominant negative and constitutively activated mutants of MEK1 have an amino acid substitution at one or more of these serines. Flag-RXR expression vector was cotransfected into HEK 293 cells with the dominant negative MEK1 mutant A217 or the constitutively active MEK1 mutant E217/E221. As shown in FIG. 4, coexpression of constitutively active MEK1 resulted in decreased mobility of Flag-RXR on SDS-PAGE, indicating that expression of active MEK1 increases phosphorylation of RXR.

These results demonstrate that the MAP kinase pathway can be activated by RXR agonists.

HEK 293 cells were cotransfected with Flag-RXR expression vector and vector alone, wild-type MEK1, constitutively active (CA), or dominant negative (DN) MEK1 mutants. The cell lysates were prepared 48 hours after transfection with or without prior stimulation of AGN194204. The lysates were subjected to separation on 4-12% SDS-PAGE, and probed with anti-RXR antibody (D20), anti-Flag antibody (M2) or anti-phospho-MAPK antibody.

### EXAMPLE II

### A Protein Kinase Interacts with RXR in a Ligand-dependent Manner

This example demonstrates that a serine-threonine kinase interacts with RXR and that this interaction is dependent upon RXR-specific ligand.

A. Ligand Dependent Association of a Kinase with the RXR Receptor

In vivo labeling with [³²P] ortho-phosphate indicated that RXR can be modified by phosphorylation. Characterization of the kinase was performed by transiently transfecting HEK 293 cells with Flag-RXR, and subsequent immunoprecipitation with anti-Flag antibody. Immunoprecipitates were used for an in vitro kinase reaction with [γ-³²P]ATP. As shown in FIG. 5A, in the presence of RXR-specific ligand, Flag-RXR immunoprecipitates contained at least one kinase that phosphorylated RXR and another protein with a molecular weight about of 160 kDa, also present in the RXR immunocomplex. This result indicated that a conformational change in RXR induced by RXR-specific ligand enhanced recruitment or activation of a kinase capable of phosphorylating RXR.

To determine whether recruitment or activation of RXR-associated kinase is modulated by ligand binding, transfected HEK 293 cells were harvested without any stimulation by RXR-specific ligand; and immunoprecipitation of Flag-RXR also was carried out in the absence of RXR-specific ligand. Subsequently, RXR-specific ligand was added into the in vitro kinase reaction mixtures. Under these experimental conditions, no kinase activity was detectable in the Flag-RXR immunoprecipitated complexes, indicating that RXR ligand binding enhances recruitment of a kinase to RXR.

Cell culture and transient transfection was performed as follows. HEK 293 cells were grown in Dulbecco's modified Eagle's medium (DMEM) supplemented with 10% fetal bovine serum (FBS), 100U of penicillin per ml and 10 μg of streptomycion per ml at 37° C. in 5% CO₂. For expression of RXR protein, 100-mm dishes of cells were transfected at 50% confluence with 2 μg of plasmid DNA using Lipofectamine (Life Technologies) as specified by the manufacturer. At 32 hours post-transfection, cells were starved by incubating in DMEM medium with 2.5% charcoal-treated fetal bovine serum or 0.1-0.5% calf bovine serum overnight, and then treated with indicated agents. Cells were harvested by aspirating medium, rinsing in phosphate buffered saline (PBS), and extracting into Lysis Buffer (30 mM Tris-HCL, pH 7.4, 0.5% Nonidet P-40, 10% glycerol, 150 mM NaCl, 1 mM EDTA, 40 mM NaF, 1 mM sodium orthovanadate, and 0.5 mM phenylmethylsufonyl fluoride, and 1×protease inhibitors, (Roche Diagnostics) or 1×SDS Sample Buffer.

Immunoprecipitation was performed essentially as follows. Cells were solubilized in cold Lysis Buffer and subsequently clarified by centrifugation at 20,000 g for 10 minutes at 4° C. Immunoprecipitations were performed at 4° C. by incubating clarified cell extracts with the indicated antibodies (2 μg/ml) and protein A/G-agarose beads (1:30 dilution of a 50% suspension) on a rotating wheel for 4 hours to overnight. Agarose beads were pelleted by low speed centrifugation and washed extensively with ice-cold Lysis Buffer.

Immunoblotting was performed as follows. Proteins from cell lysates or immunoprecipitates were subjected to separation on SDS-PAGE. The resolved polypeptides were transferred to PVDF membrane, and blocked with 10% non-fat dried milk or 3% BSA in PBS with 0.05% Tween-20 (PBST). Membranes were then incubated with primary antibodies for two hours at room temperature or overnight at 4° C. After removal of unbound antibodies, membranes were incubated with horseradish peroxidase-conjugated second antibodies for 1 hour at room temperature and washed three times with PBST. Detection was performed using the enhanced chemiluminescence immunodetection system (Amersham) according to the manufacturer's instructions.

Protein kinase assays were performed as follows. Immunoprecipitated complexes were resuspended in 15 μl of Kinase Assay Buffer (30 mM Tris-HCl, pH 7.4 and 10 mM MgCl₂). Kinase reactions were initiated by addition of 2.5 μl of 50 μM [γ-³²P]ATP (10 μCi) to the immune-complexes, followed by incubation at 25° C. with occasional shaking for 20 minutes, and terminated by addition of 6 μl 4×SDS Sample Buffer. After heating at 100° C. for 5 minutes, the reaction mixtures were resolved by 4-12% SDS-PAGE. Incorporation of ³²P into RXR or other proteins was determined by autoradiography.

B. A Serine-threonine Kinase is Associated with RXR

In order to characterize the kinase that associates with RXR, in vitro [γ-³²P]ATP labeled RXR and 160 kDa protein were separated by SDS-PAGE, transferred to PVDF membrane, and excised for phosphoamino acid analysis. As shown in FIG. 5B, RXR and 160 kDa protein each was phosphorylated on both serine and threonine residues but not on tyrosine, indicating that the kinase associated with RXR is a member of the serine/threonine kinase family. Furthermore, in vitro kinase reactions were performed in the presence of inhibitors for protein kinase A, protein kinase C, and calmodulin-dependent protein kinases. However, no inhibitory effect was detected with any of these kinase inhibitors. In sum, these results indicate that the RXR-associated kinase is a serine/threonine kinase but is not one of the known kinases, protein kinase A, protein kinase C or the calmodulin-dependent kinase.

Phosphorylated proteins were separated on SDS-PAGE and transferred to PVDF membrane essentially as described above. ³²P-labeled protein bands were identified by autoradiography, excised, and then subjected to acid hydrolysis (6 N HCl) under reduced pressure at 100° C. for 2 hours. The hydrolysates were lyophilized and washed twice with distilled water. The amino acid residues were mixed with standard P-Ser, P-Thr, and P-Tyr (Sigma) and subjected to separation by thin-layer chromatography on cellulose plates in an ascending solvent containing isobutyric acid (0.5 M NH4OH (5:3, v/v)), as described by Neufeld et al., Anal. Biochem. 177:138-143 (1989). Standards (P-Ser, P-Thr, and P-Tyr) were visualized by spraying ninhydrin onto the plate; radiolabeled amino acids were identified by autoradiography.

C. Interaction of RXR with the Associated Kinase is Dependent Upon RXR-specific Agonist

As shown in FIG. 6A, interaction of RXR with the kinase was dose-dependent. The EC50 of the AGN194204 for RXR recruiting the kinase was determined to be about 0.5 μM, which is higher than the dosage required for direct transcriptional effects.

To verify that coimmunoprecipitation of the kinase with RXR is dependent on the presence of RXR-specific ligand, cells were treated with several different RXR-specific agonists, an RXR antagonist, and an RAR agonist prior to immunoprecipitation with anti-Flag antibody. As shown in FIG. 6B, in the presence of RXR-specific ligands AGN194204, AGN195029, AGN192620, AGN195203, and AGN195184, Flag-RXR immunocomplexes from transfected HEK 293 cells contained a kinase phosphorylated RXR and a 160 kDa protein. In contrast, in the presence of RXR-specific antagonist AGN195393 or the RAR-specific agonist TTNPB, neither RXR nor the 160 kDa protein was phosphorylated. These results demonstrate that association or activation of the kinase is dependent upon RXR-specific agonist.

TABLE 4

| | |
|---|---|
| ATRA | 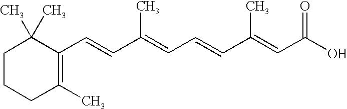 |
| 9-cis RA | 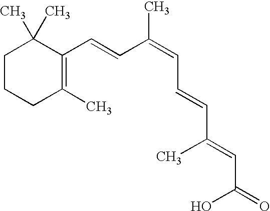 |
| TTNPB | 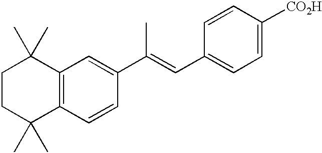 |
| AGN192620 | 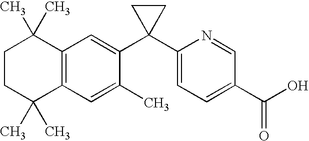 |
| AGN194204 | 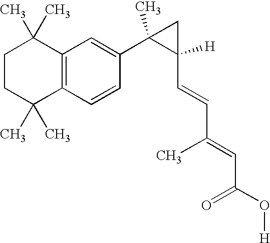 |
| AGN195029 | 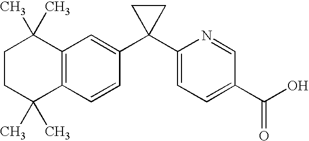 |
| AGN195183 | 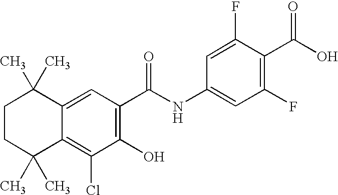 |
| AGN195184 | 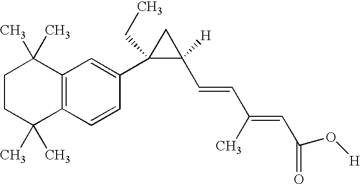 |
| AGN195203 | 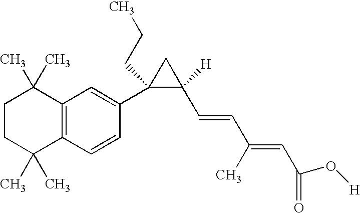 |
| AGN195393 | 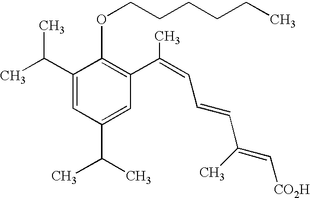 |

### EXAMPLE III

### Cellular Localization and Activity of Several RXR Deletion Mutants

This example demonstrates that the ligand-binding domain of RXR is sufficient to recruit kinase activity to an RXR-containing immunocomplex.

A. Cellular Localization of RXRα and Several Deletion Mutants

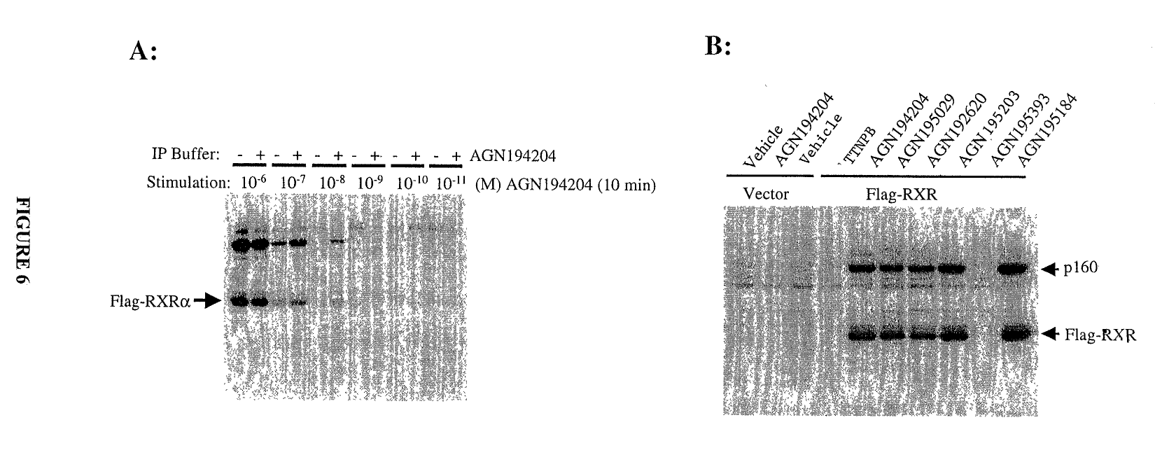

Cellular localization of RXRα proteins was assessed in HEK 293 cells transfected with expression vectors encoding RXRα, RXRαCDE (containing only the central DNA-binding domain (“C”), the hinge region (“D”), and the ligand-binding domain “E”); RXRαDE containing only domains “D” and “E”; RXRαE containing only domain “E”, and RXRαΔC containing the transcriptional activation domain (“A/B”) as well as domains “D” and “E”). Each of the recombinant proteins were tagged with an antigenic peptide, Flag (see FIG. 7). Cells were stained with commercially available antibodies against the Flag epitope, and cell nuclei were stained with Sytox Green prior to confocal microscopy. The immunostaining demonstrated that wild-type RXRα proteins were localized to nuclear and cytoplasm compartments, while each of the RXRα mutant proteins RXRα CDE, RXRαDE, RXRαE, and RXRαΔC predominantly localized to the cytoplasmic compartment.

RXRα mutants were constructed as follows. Human RXRα mutants were constructed by PCR amplification of hRXRα cDNA using paired primers specific for different regions (see Table 5). PCR reactions were performed using 0.1 μg RXRα cDNA and 100 μg of each primer in the presence of 5U AmpliTaq polymerase (Perkin Elmer). The reactions were initiated at 94° C. for 5 minutes and followed by 40 cycles of amplification (30 seconds at 58° C.; 30 seconds at 94° C.; and 1 minute at 72° C.) and extended for 7 minutes at 72° C. The resulting PCR fragments were digested with EcoRI and KpnI and cloned into a CMV-Flag vector using standard techniques. For constructing RXRαΔC, the EcoRI fragment from PCR, which contains the A/B region of RXRα, was inserted into RXRαDE at the EcoRI site in front of the DE region of RXRα.

TABLE 5
PCR oligonucleotide primers for hRXRα mutants

| | | SEQ |
|---|---|---|
| | | ID |
| Mutant | PCR Paired Primers | NO. |
| RXRα | C1: | 52 |
| CDE | 5′-AGGAATTCTGCGCCATCTGCGGGGACCGC-3′ | |
| | C4: | 55 |
| | 5′-AGGGTACCCTAACTCATTTGGTGCGGCGCCTCC-3′ | |
| RXRα | C2: | 53 |
| DE | 5′-AGGAATTCAAGCGGGAAGCCGTGCAGGAGGAGCGG-3′ | |
| | C4: | 55 |
| | 5′-AGGGTACCCTAAGTCATTTGGTGCGGCGCCTCC-3′ | |
| RXRα | C3: | 54 |
| E | 5′-AGGAATTCTCGCCGAACGACCCTGTCACC-3′ | |
| | C4: | 55 |
| | 5′-AGGGTACCCTAAGTCATTTGGTGCGGCGCCTCC-3′ | |
| RXRα | C19: | 57 |
| ΔAF2 | 5′-AGGAATTCATGGACACCAAACATTTCCTGCCG-3′ | |
| | C5: | 56 |
| | 5′-AGGGTACCCTAGATGAGCTTGAAGAAGAAGAG-3′ | |
| RXRα | C1: | 52 |
| CDEΔAF | 5′-AGGAATTCTGCGCCATCTGCGGGGACCGC-3′ | |
| 2 | | |
| | C5: | 56 |
| | 5′-AGGGTACCCTAGATGAGCTTGAAGAAGAAGAG-3′ | |
| RXRα | C2: | 53 |
| DEΔAF2 | 5′-AGGAATTCAAGCGGGAAGCCGTGCAGGAGGAGCGG-3′ | |
| | C5: | 56 |
| | 5′-AGGGTACCCTAGATGAGCTTGAAGAAGAAGAG-3′ | |
| RXRα | C3: | 54 |
| EΔAF2 | 5′-AGGAATTCTCGCCGAACGACCCTGTCACC-3′ | |
| | C5: | 56 |
| | 5′-AGGGTACCCTAGATGAGCTTGAAGAAGAAGAG-3′ | |
| RXRαΔC | C19: | 57 |
| | 5′-AGGAATTCATGGACACCAAACATTTCCTGCCG-3′ | |
| | C55: | 58 |
| | 5′-AGGAATTCGATGTGCTTGGTGAAGGAAGCC-3′ | |

Immunofluorescence was performed essentially as follows. 293 cells grown in DMEM/10% fetal bovine serum (FBS) were seeded at 15,000 cells/well on 24-well plate containing cover slips coated with Poly-D-Lysine (Becton Dickinson; Franklin Lakes, N.J.). The next day, cells were transfected with RXRα or mutant expression vector using Lipofectamine (Life Technologies; Rockville, Md.), and after five hours, fresh medium was added. The following day, cells were treated with appropriate ligands for 10 minutes and subsequently stained with mouse monoclonal antibodies against Flag (M2; SIGMA, St. Louis, Mich.) and HRP-conjugated anti-mouse IgG (Jackson Immunoresearch Laboratories; West Grove, Pa.) according to the protocol provided with kit TSA Direct (New England Nuclear; Boston, Mass.). HRP activity was revealed using substrates coupled to tetramethylrhodamine. Cell nuclei were stained with 0.5-2.5 μM Sytox Green (Molecular Probes; Eugene, Oreg.). Stained cells were photographed using a confocal microscope (Leica Microsystems; Bannockburn, Ill.).

B. The Ligand-binding Domain of RXR is Sufficient for Association of the Protein Kinase

Various deletion mutants of RXRα were transfected and used for an in vitro kinase reaction, as described above for full-length RXR. All mutants containing an intact E region, comprising the critical core of the ligand-binding domain, recruited kinase activity to the immunocomplex in the presence of AGN194204 (see FIG. 8). However, kinase activity was not recruited in the presence of AGN194204 upon deletion of the AF-2 region from RXRα (FIG. 8). These results demonstrate that the ligand-binding domain, including transactivation domain 2 (AF-2) is necessary and sufficient for ligand-dependent association with a kinase.

### EXAMPLE IV

### A Heat Shock Protein Inhibitor Increases Kinase Activity Associated with RXR

This example demonstrates that RXR can be associated with heat shock proteins and that inhibition of heat shock protein 90 (HSP90) can result in increased kinase activity associated with RXR.

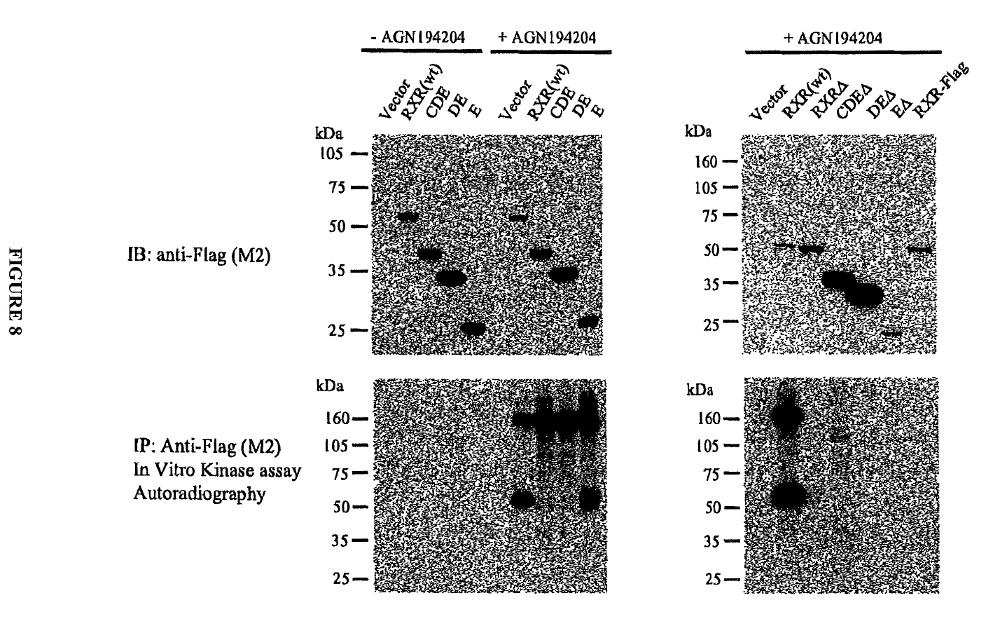

Flag-RXR immunocomplexes from transfected HEK 293 cells isolated using anti-Flag antibody (M2) were separated using two-dimensional gel electrophoresis. As shown in FIG. 9, a group of proteins having molecular weights of about 70-75 kDa (indicated by circle) were pulled down together with Flag-RXR from HEK 293 cells transfected with Flag-RXR. These proteins were not pulled down from HEK 293 cells transfected with control vector. Four protein spots (inside the circle) were isolated, and trypsin-hydrolyzed fragments of these proteins were analyzed by mass spectrometry. Each of these proteins was identified as a member of the heat shock protein 70 (HSP70) family of proteins. These results demonstrate ligand-specific interaction of heat shock proteins with the RXR receptor.

Two-dimensional gel electrophoresis was carried out according to the procedure of O'Farrell et al., J. Biol. Chem. 250:4007-4021 (1975). The first dimensional separation was obtained by isoelectric focusing (IEF) or non-equilibrium pH gradient electrophoresis (NEPHGE). Following first dimensional separation, the gel was equilibrated with 10% glycerol, 5% 2-ME, 2% SDS and 62.5 mM Tris-HCl (pH 6.8), and subjected to the separation on second dimensional SDS-PAGE.

The nuclear receptor RXR can interact with HSP70 and HSP90 as shown above. To analyze the effect of inhibiting heat shock protein activity, RXR transfected HEK 293 cells were treated with the HSP90 inhibitor geldanamycin overnight. As shown in FIG. 9, this treatment dramatically changed the mobility of RXR in SDS-PAGE (bottom panel) and significantly increased the kinase activity in the RXR immunoprecipitated complex. These results demonstrate that heat shock proteins such as heat shock protein 90 can negatively regulate RXR-associated kinase activity.

### EXAMPLE V

### Ligand-dependent Protein Kinase A Activity is Associated with RXRα

A. Protein Kinase A activity Associated with Hormone Receptor

This example demonstrates that protein kinase A activity is associated with RXRα and displays ligand dependence.

HEK 293 cells transfected with an expression vector encoding a PH-RXRα fusion protein were treated with or without 10⁻⁷ M of the RXRα ligand, AGN194204. Following immunoprecipitation with anti-Flag antibody, the synthetic PKA substrate, LRRASLG (SEQ ID NO: 59), was added to the immunoprecipitate along with γ-ATP. As shown in FIG. 14A (lanes 1 to 4), significantly more protein kinase A activity was detected in immunoprecipitates from cells treated with AGN194024 ligand than in immunoprecipitates treated with control vehicle. Furthermore, this activity was specifically inhibited by TYADFIASGRTGRRNAI (SEQ ID NO: 142), an inhibitor of protein kinase A, although not by an inhibitor of protein kinase C (RFARKGALRGKNV; SEQ ID NO: 143). See FIG. 14A, lanes 5 and 6. In the absence of peptide substrate, the amount of ³²P-incorporation was markedly reduced (see lanes 7 and 8).

Similar results were obtained when PKA activity associated with RXRα was assayed with another synthetic PKA substrate, GRTGRRNSI (SEQ ID NO: 60), as shown in FIG. 14B. At least twice as much protein kinase A activity was observed in immunoprecipitates prepared with anti-FLAG antibody from cells treated with AGN194204 as compared to control DMSO-treated cells. These results indicate that, in assays with a second PKA substrate, PKA activity associated with RXRα was regulated by receptor ligand.

Similar results were obtained in HEK 293 cells transfected with Flag-RXRα that did not contain the pleckstrin homology domain. Following treatment of transfected cells with AGN194204 or control vehicle, RXRα-associated kinase activity was co-immunoprecipitated with anti-Flag antibody, and kinase activity assayed using synthetic substrate LRRASLG (SEQ ID NO: 59) as described above. As shown in FIG. 14C, protein kinase A activity associated with RXRα lacking a fused pleckstrin homology domain was significantly enhanced in cells treated with 10⁻⁷ M AGN194204 as compared to control vehicle (see lanes 5 and 6). Although expression levels of RXRα and PH-RXRα were similar, the ligand-dependent increase observed with RXRα was not as great as the increase observed in PH-RXRα transfected cells (compare lanes 5 and 6 to lanes 1 and 2).

The Flag-tagged PH-RXRα was prepared essentially as follows. The pleckstrin homology domain of human insulin receptor substrate-1 (corresponding to residues 12 to 115 of GenBank Accession No. S62539) was synthesized by shot-gun ligation of complimentary oligodeoxyribonucleotides: 5′-AG CTT GAC GTG CGC AAA GTG CGC TAC CTG CGC AAA CCC AAG AG-3′ (SEQ ID NO: 144); 5′-CA TGC ACA AAC GCT TCT TCG TAC TGC GCG CGG CCA GCG AGG CTG GGG GC-3′ (SEQ ID NO: 145); 5′-CC GGC GCG CCT CGA CTA GTA CGA GAA CGA GAA GAA GTC GCG GCA CA-3′ (SEQ ID NO: 146); 5′-AG TCG AGC GCC CCC AAA CGC TCG ATC CCC CTT GAG AGC TGC TTC AA-3′ (SEQ ID NO: 147); 5′-CA TCA ACA AGC GGG CTG ACT CCA AGA ACA AGC ACC TGG TGG CTC TC-3′ (SEQ ID NO: 148); 5′-TA CAC CCG GGA CGA GCA CTT TGC CAT CGC GGC GGA CAG CGA GGC CGA GCA AGA C-3′ (SEQ ID NO: 149); 5′-AG CTG GTA CCA GGC TCT CCT ACA GCT GCA CAA CG-3′ (SEQ ID NO: 150); 5′-AA TTC GTT GTG CAG CTG TAG GAG AGC CTG GTA CCA GCT GTC TTG CTC GGC CTC-3′ (SEQ ID NO: 151); 5′-GC TGT CCG CCG CGA TGG CAA AGT GCT CGT CCC GGG TGT AGA GAG CCA CCA GGT-3′ (SEQ ID NO: 152); 5′-GC TTG TTC TTG GAG TCA GCC CGC TTG TTG ATG TTG AAG CAG CTC TCA AGG GGG-3′ (SEQ ID NO: 153); 5′-AT CGA GCG TTT GGG GGC GCT CGA CTT GTG CCG CCA CTT CTT CTC GTT CTC GTA-3′ (SEQ ID NO: 154); 5′-GT ACT CGA GGC GCG CCG GGC CCC CAG CCT CGC TGG CCG CGC GCA GTA CGA AGA-3′ (SEQ ID NO: 155); and 5′-AG CGT TTG TGC ATG CTC TTG GGT TTG CGC AGG TAG CCC ACC TTG CGC ACG TCA-3′ (SEQ ID NO: 156). The resulting fragment was inserted into the Hind III and Eco RI sites between the Flag tag and RXRα coding regions in Flag-RXRα.

HEK 293 were cultured and transfected essentially as described above. Immunoprecipitates were prepared using the anti-Flag antibody, M2, as described above. Assays for protein kinase A activity were performed as follows. Immunoprecipitated complexes were resuspended in 40 μl of Kinase Assay Buffer (30 mM Tris-HCl, pH 7.4 and 10 mM MgCl₂). Kinase reactions were initiated by addition of 10 μl of 500 μM synthetic peptide LRRASLG (SEQ ID NO: 59) or GRTGRRNSI (SEQ ID NO: 60) prepared by Upstate Biotechnology (Lake Placid, N.Y.) and addition of 50 μM [γ-³²P]ATP (10 μCi) to the immune-complexes. The reaction mixtures were then incubated at 25° C. with occasional shaking for 20 minutes, and terminated by addition of 30 μl of 40% TCA. Where indicated, 10 μl of 6 μM protein kinase A inhibitor TYADFIASGRTGRRNAI (SEQ ID NO: 142; Upstate Biotechnology) was added to the kinase reaction with the substrate and [γ-³²P]ATP. Similarly, where indicated 10 μl of 2 μM protein kinase C inhibitor RFARKGALRGKNV (SEQ ID NO: 143; Upstate Biotechnology) and 20 μM of CaMDK compound R24571 (Upstate Biotechnology) were added to the kinase reaction.

Incorporated ³²P was separated from free label by applying the reaction mixture onto P81 phosphocellulose paper and subsequently washing the paper three times with 0.75% phosphoric acid and once with acetone. Protein kinase reaction samples were analyzed in a scintillation counter to determine the amount of ³²P-incorporation.

B. Ligand Dependence of Protein Kinase A Activity Associated with Endogenous Hormone Receptor

This example demonstrates that protein kinase A activity associated with endogenous RXRα also was RXR ligand-dependent.

Untransfected 3T3-L1 adipocytes cultured essentially as described (Student et al., J. Biol. Chem. 255:4745-4750 (1980)) were treated with 0.1% DMSO or 10⁻⁷ M AGN194204, and immunoprecipitates prepared with anti-RXRα antibody D20 as described above. Kinase activity in the immunoprecipitates was determined as described above using γ-ATP and synthetic peptide SEQ ID NO: 59. As shown in FIG. 15A, PKA activity associated with endogenous RXRα in 3T3-L1 adipocytes increased about two-fold in cells treated with RXRα ligand as compared to vehicle-treated cells (compare lanes 1 and 2). These results demonstrate that protein kinase A activity associated with endogenous RXRα is regulated by hormone receptor ligand.

C. PKA Activity in Cells Overexpressing the Catalytic Subunit of PKA

This example demonstrates that, in cells overexpressing the catalytic subunit of protein kinase A, there is an enhanced RXR ligand-dependent increase in phosphorylation of PKA substrates.

S293 cells (HEK293 cells stably overexpressing RXRα) were transfected with the pFC-PKA expression vector encoding the catalytic subunit of protein kinase A and with Flag-tagged PH-RXRα essentially as described above. This commercially available expression vector expresses the catalytic subunit of murine protein kinase A under control of the CMV promoter (Stratagene; San Diego, Calif.). Transfected S293 cells were treated with or without AGN194204 and immunoprecipitated with or without anti-Flag antibody prior to assaying for protein kinase A activity in the immunoprecipitates with synthetic PKA substrate SEQ ID NO: 59. Immunoprecipitates prepared with anti-Flag antibody showed a dramatic increase in the amount of protein kinase A activity (see FIG. 15B; compare lanes 3 and 4). In contrast, immunoprecipitates prepared with anti-PKA antibody showed only a marginal ligand-dependent increase, indicating that RXR ligand-dependent regulation of protein kinase A activity is mostly or entirely confined to protein kinase A physically associated with RXR receptor.

These results indicate that the increase in RXRα associated protein kinase A activity produced by receptor ligand can be enhanced in cells overexpressing the catalytic subunit of protein kinase A.

### EXAMPLE VI

### Ligand-dependent Protein Phosphorylation by an RXR-associated Protein Kinase

A. In vitro Phosphorylation of Myelin Basic Protein and Histone-M

This example demonstrates that purified protein substrates for protein kinase A can be phosphorylated in vitro by a protein kinase associated with RXRα.

HEK 293 cells transfected with Flag-RXR were treated with or without 10⁻⁷ M AGN194204, and RXR-containing immunoprecipitates prepared using anti-Flag antibody as described above. γ-ATP and the indicated purified protein were added to immunoprecipitated material in kinase reaction buffer. After terminating the kinase reaction, the products were analyzed by SDS-PAGE. As shown in FIG. 16, in vitro phosphorylation of myelin basic protein (MBP) by RXR-associated kinase was dramatically increased in Flag-RXR transfected cells treated with AGN194204. Tn vitro phosphorylation of histone-M also increased in cells treated with the RXRα ligand.

Kinase reactions with purified proteins were performed essentially as described above for synthetic peptide substrates. Briefly, immunoprecipitated complexes were resuspended in kinase assay buffer with 2 μg myelin basic protein (Sigma) or histone-M (Roche), and kinase reactions initiated by addition of 50 μM [γ-³²P]ATP (10 μCi). The reaction mixtures were then incubated at 25° C. with occasional shaking for 20 minutes, and terminated by addition of sample buffer. SDS-PAGE analysis and autoradiography were used to visualize the results.

B. Direct, Stable Associated Between RXRα and Catalytic Subunit of Protein Kinase A

This example demonstrates a ligand-independent in vivo association between RXRα and PKA in cells overexpressing the catalytic subunit of protein kinase A.

Flag-RXRα transfected cells treated with or without 10⁻⁷ M AGN194204 were immunoprecipitated with anti-Flag antibody and subsequently blotted with anti-protein kinase A antibody. In a parallel experiment, transfected cells were immunoprecipitated with anti-protein kinase A antibody and subsequently blotted with anti-Flag antibody. As shown in FIG. 17A, protein kinase A was directly associated with RXRα in transfected cells, although treatment with AGN194204 did not increase the amount of protein kinase A associated with receptor.

Immunoprecipitations with anti-Flag antibody were performed as described above. Immunoprecipitations with anti-PKA antibody (Transduction Laboratories; Lexington, Ky.) were performed according to manufacturer's protocol. Western blotting with anti-PKA antibody and anti-Flag antibody also were performed according to manufacturer's protocol.

These results indicate that protein kinase A can directly associate with RXRα in cells overexpressing the catalytic subunit of protein kinase A in the absence of receptor ligand. These results further indicate that hormone ligand can regulate the activity of protein kinase A associated with hormone receptor. It is understood that, under other conditions such as balanced expression of the regulatory and catalytic subunits, association of receptor with protein kinase A also can be regulated by receptor ligand.

C. RXRα is Phosphorylated by Protein Kinase A In vitro

This example demonstrates that RXRα is phosphorylated by protein kinase A in vitro and that this phosphorylation is enhanced in cells treated with RXR ligand.

Flag-RXRα transfected HEK293 cells were incubated in the presence or absence of 10⁻⁷ M AGN194204 as described above. RXRα-containing immunoprecipitates prepared with anti-Flag antibody were incubated with purified catalytically active PKA (Upstate Biotechnology) and γ-ATP; in addition, protein kinase A inhibitor or protein kinase C inhibitor was added to some reactions as indicated. The kinase reaction products were analyzed by SDS-PAGE. As shown in FIG. 17B, RXRα was phosphorylated, and the extent of phosphorylation was increased by addition of RXRα ligand, AGN194204. Furthermore, phosphorylation was blocked by addition of the protein kinase A inhibitor, TYADFIASGRTGRANAI (SEQ ID NO: 142), but not by addition of a protein kinase C inhibitor.

These results indicate that RXRα can be phosphorylated by protein kinase A and that this phosphorylation can be enhanced by RXRα ligand.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. A method for identifying an effective agent that modulates a biological activity of a nuclear hormone receptor, comprising the steps of:
(a) contacting said nuclear hormone receptor with one or more agents and a eukaryotic cell sample to form a test sample under conditions suitable to form a receptor-containing complex;
(b) isolating said receptor-containing complex from said test sample;
(c) providing to said isolated receptor-containing complex conditions suitable for modification of said receptor-containing complex; and
(d) assaying said isolated receptor-containing complex for an altered modification state occurring in said isolated receptor-containing complex as compared to a control modification state,
wherein the presence of said altered modification state indicates that at least one of said one or more agents is an effective agent that modulates a biological activity of the nuclear hormone receptor.

2. A method for identifying an effective agent that modulates a biological activity of a nuclear hormone receptor, comprising the steps of:
(a) contacting said nuclear hormone receptor with one or more agents and a eukaryotic cell sample to form a test sample under conditions suitable to form a receptor-containing complex;
(b) isolating said receptor-containing complex from said test sample;
(c) providing to said isolated receptor-containing complex conditions suitable for phosphorylation of said receptor-containing complex; and
(d) assaying said isolated receptor-containing complex for an altered phosphorylation state occurring in said isolated receptor-containing complex as compared to a control phosphorylation state,
wherein the presence of said altered phosphorylation state indicates that at least one of said one or more agents is an effective agent that modulates a biological activity of the nuclear hormone receptor.

3. The method of claim 2, wherein said altered phosphorylation state is an increased phosphorylation state.

4. The method of claim 2, wherein said altered phosphorylation state is a decreased phosphorylation state.

5. A method for identifying an effective agent that modulates a biological activity of a nuclear hormone receptor, comprising the steps of:
(a) contacting said nuclear hormone receptor with one or more agents and a eukaryotic cell sample to form a test sample under conditions suitable to form a receptor-containing complex;
(b) isolating said receptor-containing complex from said test sample;
(c) providing to said isolated receptor-containing complex conditions suitable for phosphorylation of said receptor-containing complex; and
(d) assaying said isolated receptor-containing complex for an altered phosphorylation state occurring in said isolated receptor-containing complex as compared to a control phosphorylation state,
wherein said altered phosphorylation state is an altered phosphorylation state of said nuclear hormone receptor or an altered phosphorylation state of a 160 kDa protein, and
wherein the presence of said altered phosphorylation state indicates that at least one of said one or more agents is an effective agent that modulates a biological activity of the nuclear hormone receptor.

6. The method of claim 5, wherein said altered phosphorylation state is an altered phosphorylation state of said nuclear hormone receptor.

7. The method of claim 5, wherein said altered phosphorylation state is an altered phosphorylation state of a 160 kDa protein.

8. The method of claim 1, wherein said one or more agents are present during step (b).

9. The method of claim 2, where said conditions suitable for modification of said receptor-containing complex are a magnesium concentration of 1 to 25 mM.

10. The method of claim 1, wherein said nuclear hormone receptor is selected from the group consisting of a
retinoid X receptor (RXR),
hepatocyte nuclear factor 4 (HNF4),
testicular receptor,
tailless gene homolog (TLX),
Chicken ovalbumin upstream promoter transcription factor (COUP-TF),
thyroid receptor (TR),
retinoic acid receptor (RAR),
peroxisome proliferator activated receptor (PPAR),
reverse Erb (revErb),
RAR-related orphan receptor (ROR), steroidogenic factor-1 (SF-1),
liver receptor homolog-1 (LRH-1),
liver X receptor (LXR),
farnesoid X receptor (FXR),
vitamin D receptor (VDR),
ecdysone receptor (EcR),
pregnane X receptor (PXR),
constitutive androstane receptor (CAR),
neuron-derived activated receptor (NOR1),
nuclear receptor related 1 (NURR1),
estrogen receptor (ER),
estrogen-related receptor (ERR),
glucocorticoid receptor (GR),
androgen receptor (AR),
progesterone receptor (PR) and
mineralocorticoid receptor (MR).

11. A method for identifying an effective agent that modulates a biological activity of a nuclear hormone receptor, comprising the steps of:
(a) contacting said nuclear hormone receptor with one or more agents and a eukaryotic cell sample to form a test sample under conditions suitable to form a receptor-containing complex,
wherein said nuclear hormone receptor is selected from the group consisting of a retinoid X receptor, retinoic acid receptor, progesterone receptor, estrogen receptor, androgen receptor and vitamin D receptor;
(b) isolating said receptor-containing complex from said test sample;
(c) providing to said isolated receptor-containing complex conditions suitable for modification of said receptor-containing complex; and
(d) assaying said isolated receptor-containing complex for an altered modification state occurring in said isolated receptor-containing complex as compared to a control modification state,
wherein the presence of said altered modification state indicates that at least one of said one or more agents is an effective agent that modulates a biological activity of the nuclear hormone receptor.

12. The method of claim 11, wherein said nuclear hormone receptor is selected from the group consisting of RARα, RARβ, RARγ, RXRα, RXRβ and RXRγ.

13. The method of claim 11, wherein said nuclear hormone receptor is a retinoid X receptor (RXR) selected from the group consisting of RXRα, RXRβ and RXRγ.

14. A method for identifying an effective agent that modulates a biological activity of a retinoid X receptor, comprising the steps of:
(a) contacting said retinoid X receptor with one or more agents and a eukaryotic cell sample to form a test sample under conditions suitable to form a retinoid X receptor-containing complex;
(b) isolating said receptor-containing complex from said test sample;
(c) providing to said isolated receptor-containing complex conditions suitable for modification of said receptor-containing complex; and
(d) assaying said isolated receptor-containing complex for an altered phosphorylation state occurring in said isolated receptor-containing complex as compared to a control phosphorylation state,
wherein the presence of said altered modification state indicates that at least one of said one or more agents is an effective agent that modulates a biological activity of said retinoid X receptor.

15. The method of claim 1, wherein said nuclear hormone receptor is exogenous to said cell.

16. The method of claim 1, wherein said nuclear hormone receptor is truncated.

17. The method of claim 16, wherein said truncated nuclear hormone receptor lacks the native DNA-binding domain.

18. The method of claim 16, wherein said truncated nuclear hormone receptor consists essentially of the ligand-binding domain.

19. The method of claim 1, wherein said nuclear hormone receptor is a variant with an increased ratio of cytoplasmic to nuclear localization as compared to wild type nuclear hormone receptor.

20. The method of claim 1, wherein said nuclear hormone receptor is a variant that lacks a functional DNA-binding domain.

21. The method of claim 1, wherein said nuclear hormone receptor is a fusion protein comprising a heterologous membrane-anchoring domain.

22. The method of claim 1, wherein said nuclear hormone receptor is a fusion protein comprising a heterologous epitope tag.

23. The method of claim 1, wherein said nuclear hormone receptor is a fusion protein comprising a heterologous protein kinase recognition sequence.

24. The method of claim 1, wherein step (b) comprises specific binding to said receptor-containing complex.

25. The method of claim 24, wherein step (b) comprises immunoprecipitation of said receptor-containing complex.

26. The method of claim 25, wherein said immunoprecipitation is performed using antibody immunoreactive with said nuclear hormone receptor or a heterologous epitope fused thereto.

27. The method of claim 26, wherein said immunoprecipitation is performed using antibody immunoreactive with said nuclear hormone receptor.

28. The method of claim 2, wherein step (c) comprises incubating said receptor-containing complex with ATP.

29. The method of claim 1, wherein said test sample comprises an exogenous heterodimeric partner of said nuclear hormone receptor.

30. The method of claim 1, wherein said test sample comprises an exogenous kinase that enhances detection of said altered modification state.

31. The method of claim 1, wherein said eukaryotic cell sample comprises viable cells.

32. The method of claim 1, wherein said eukaryotic cell sample is a whole cell lysate.

33. The method of claim 1, wherein said eukaryotic cell sample is a fractionated cell lysate.

34. The method of claim 1, wherein said receptor-containing complex comprises a serine/threonine kinase.

35. A method for identifying an improved effective agent that modulates a biological activity of a nuclear hormone receptor, comprising the steps of:
(a) contacting said nuclear hormone receptor with one or more agents and a eukaryotic cell sample to form a test sample under conditions suitable to form a receptor-containing complex;
(b) isolating said receptor-containing complex from said test sample;
(c) providing to said isolated receptor-containing complex conditions suitable for modification of said receptor-containing complex,
(d) assaying said isolated receptor-containing complex for an altered modification state occurring in said isolated receptor-containing complex as compared to a control modification state; and
(e) assaying for direct transcriptional activity of said nuclear hormone receptor contacted with said one or more agents,
wherein the presence of said altered modification state combined with the absence of said direct transcriptional activity indicates that at least one of said one or more agents is an improved effective agent that modulates a biological activity of the nuclear hormone receptor.

36. A method for identifying an effective agent that modulates protein kinase A activity associated with a nuclear hormone receptor, comprising the steps of:
(a) contacting said nuclear hormone receptor with one or more agents and a cell sample to form a test sample under conditions suitable to form a receptor-containing complex;
(b) isolating said receptor-containing complex from said test sample;
(c) contacting said isolated receptor-containing complex with a protein kinase A substrate under conditions suitable for phosphorylation of said substrate; and
(d) assaying said substrate for an altered phosphorylation state as compared to a control phosphorylation state,
wherein the presence of said altered phosphorylation state indicates that at least one of said one or more agents is an effective agent that modulates protein kinase A activity associated with said nuclear hormone receptor.

37. The method of claim 36, wherein said altered phosphorylation state is an increased phosphorylation state.

38. The method of claim 36, wherein said altered phosphorylation state is a decreased phosphorylation state.

39. The method of claim 36, wherein said substrate is a purified substrate.

40. A method for identifying an effective agent that modulates protein kinase A activity associated with a nuclear hormone receptor, comprising the steps of:
(a) contacting said nuclear hormone receptor with one or more agents and a cell sample to form a test sample under conditions suitable to form a receptor-containing complex;
(b) isolating said receptor-containing complex from said test sample;
(c) contacting said isolated receptor-containing complex with a purified protein kinase A peptide substrate under conditions suitable for phosphorylation of said substrate; and
(d) assaying said peptide substrate for an altered phosphorylation state as compared to a control phosphorylation state,
wherein the presence of said altered phosphorylation state indicates that at least one of said one or more agents is an effective agent that modulates protein kinase A activity associated with said nuclear hormone receptor.

41. The method of claim 40, wherein said peptide substrate comprises a sequence selected from the group consisting of Arg-X-Ser, Arg-Arg-X-Ser, Arg-X-X-Ser, Lys-Arg-X-X-Ser and Arg-X-Lys-Arg-X-X-Ser-X (SEQ ID NO: 113), where X is independently any amino acid.

42. The method of claim 41, wherein said peptide substrate comprises the amino acid sequence Arg-X-Ser, where X is independently any amino acid.

43. The method of claim 42, wherein said peptide substrate comprises the amino acid sequence Arg-Arg-X-Ser, where X is independently any amino acid.

44. The method of claim 41, wherein said peptide substrate comprises the amino acid sequence Lys-Arg-X-X-Ser, where X is independently any amino acid.

45. The method of claim 44, wherein said peptide substrate comprises the amino acid sequence Arg-X-Lys-Arg-X-X-Ser-X (SEQ ID NO: 113), where X is independently any amino acid.

46. The method of claim 43, wherein said peptide substrate comprises an amino acid sequence selected from the group consisting of LRRASLG (SEQ ID NO: 59) and GRTGRRNSI (SEQ ID NO: 60).

47. The method of claim 40, 41, 42, 43, 44, 45 or 46, wherein said peptide substrate has at most ten residues.

48. The method of claim 39, wherein said purified substrate is myelin basic protein.

49. The method of claim 39, wherein said purified substrate has a Km of less than 20 μM for protein kinase A.

50. A method for identifying an effective agent that modulates protein kinase A activity associated with a nuclear hormone receptor, comprising the steps of:
(a) contacting said nuclear hormone receptor with one or more agents and a cell sample to form a test sample under conditions suitable to form a receptor-containing complex,
wherein said nuclear hormone receptor is selected from the group consisting of a retinoid X receptor (RXR), retinoic acid receptor (RAR) and a peroxisome proliferator activated receptor (PPAR);
(b) isolating said receptor-containing complex from said test sample;
(c) contacting said isolated receptor-containing complex with a protein kinase A substrate under conditions suitable for phosphorylation of said substrate; and
(d) assaying said substrate for an altered phosphorylation state as compared to a control phosphorylation state,
wherein the presence of said altered phosphorylation state indicates that at least one of said one or more agents is an effective agent that modulates protein kinase A activity associated with said nuclear hormone receptor.

51. The method of claim 50, wherein said nuclear hormone receptor is a retinoid X receptor (RXR).

52. The method of claim 51, wherein said retinoid X receptor is RXRα.

53. The method of claim 36, 51 or 52, wherein said nuclear hormone receptor is a variant with an increased ratio of cytoplasmic to nuclear localization as compared to wild type nuclear hormone receptor.

54. The method of claim 36, 51 or 52, wherein said nuclear hormone receptor is a fusion protein comprising a heterologous membrane-anchoring domain.

55. The method of claim 36, 51 or 52, wherein said nuclear hormone receptor is a fusion protein comprising a pleckstrin homology domain.

56. The method of claim 36, 51 or 52, wherein said nuclear hormone receptor is a fusion protein comprising a heterologous epitope tag.

57. The method of claim 36, 51 or 52, wherein said cell sample comprises an exogenous nucleic acid molecule encoding said nuclear hormone receptor.

58. The method of claim 36, 51 or 52, wherein said nuclear hormone receptor is endogenous to said cell sample.

59. The method of claim 36, 51 or 52, wherein said cell sample comprises an exogenous nucleic acid molecule encoding a heterodimeric partner of said nuclear hormone receptor.

60. The method of claim 36, 51 or 52, wherein said cell sample comprises an exogenous nucleic acid molecule encoding a catalytic subunit of protein kinase A.

61. The method of claim 36, wherein said cell sample comprises viable eukaryotic cells.

62. The method of claim 36, wherein said cell sample is a eukaryotic whole cell lysate.

63. The method of claim 36, wherein said one or more agents are present during step (b).

64. The method of claim 36, wherein step (b) comprises specific binding to said receptor-containing complex.

65. A method for identifying an effective agent that modulates protein kinase A activity associated with a nuclear hormone receptor, comprising the steps of:
(a) contacting said nuclear hormone receptor with one or more agents and a cell sample to form a test sample under conditions suitable to form a receptor-containing complex;
(b) immunoprecipitating said receptor-containing complex from said test sample, thereby isolating said receptor-containing complex;
(c) contacting said isolated receptor-containing complex with a protein kinase A substrate under conditions suitable for phosphorylation of said substrate; and
(d) assaying said substrate for an altered phosphorylation state as compared to a control phosphorylation state,
wherein the presence of said altered phosphorylation state indicates that at least one of said one or more agents is an effective agent that modulates protein kinase A activity associated with said nuclear hormone receptor.

66. The method of claim 65, wherein said immunoprecipitation is performed using antibody immunoreactive with said nuclear hormone receptor.

67. The method of claim 65, wherein said nuclear hormone receptor is a fusion protein comprising a heterologous epitope tag and said immunoprecipitation is performed using antibody immunoreactive with said epitope tag.

68. The method of claim 65, wherein step (d) comprises detecting radiolabeled substrate.
